(19) United States
(12) Patent Application Publication
(10) Pub. No.: US 2010/0160324 A1
(43) Pub. Date: Jun. 24, 2010

(54) PYRAZOLE DERIVATIVES AS THAT MODULATE THE ACTIVITY OF CDK, GSK AND AURORA KINASES

(75) Inventors: Valerio Berdini, Cambridge (GB); Michael Alistair O'Brien, Hitchin (GB); Maria Grazia Carr, Luton (GB); Nicholas Gareth Morse Davies, Cambridge (GB); Adrian Liam Gill, Caldecote (GB); Eva Figueroa Navarro, Cambridge (GB); Steven Howard, Cambridge (GB); Gary Trewartha, Stevenage (GB); Andrew James Woodhead, Cambridge (GB); Alison Jo-Anne Woolford, Cambridge (GB); Paul Graham Wyatt, Perth (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS LIMITED, Cambridge (GB)

(21) Appl. No.: 11/813,167

(22) PCT Filed: Dec. 30, 2005
(86) PCT No.: PCT/GB2005/005102

§ 371 Date: Mar. 11, 2010

Related U.S. Application Data
(60) Provisional application No. 60/640,474, filed on Dec. 30, 2004.

(30) Foreign Application Priority Data
Dec. 30, 2004 (GB) 0428528.4

(51) Int. Cl. C07D 403/04; A61P 25/28; C07D 413/04; A61K 31/497; A61K 31/4353; A61K 31/4439; A61K 31/4245; A61K 31/427; A61K 31/422; A61K 31/4196; A61K 31/415; A61K 31/5377; C07D 471/04; C07D 401/04; C07D 417/04; C07D 249/12; A61K 31/4155; A61K 31/496; C07D 231/10; A61P 35/04

(52) U.S. Cl. 514/236.2; 514/254.05; 514/255.05; 514/300; 514/341; 514/364; 514/365; 514/374; 514/383; 514/397; 514/404; 544/140; 544/371; 544/405; 546/121; 546/275.4; 548/143; 548/204; 548/236; 548/263.2; 548/312.4; 548/364.7; 548/365.4; 548/372.1

(57) ABSTRACT

The invention provides a compound of the formula (I): for use in medicine: or salts or tautomers or N-oxides or solvates thereof; wherein R¹ is an optionally substituted heterocyclic group having from 3 to 12 ring members provided that the cyclic group joined to the pyrazole contains at least one heteroatom selected from N, O or S; A is a bond or —Y—(B)ₙ—; B is C═O, NR^{g}(C═O) or O(C═O) wherein R^{g} is hydrogen or C₁₋₄ hydrocarbyl optionally substituted by hydroxy or C₁₋₄ alkoxy; n is 0 or 1; Y is a bond or an alkylene chain of 1,2 or 3 carbon atoms in length; R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy); R³ is selected from optionally substituted carbocyclic and heterocyclic groups having from 3 to 12 ring members or an optionally substituted C₁₋₈ hydrocarbyl group; with the proviso that R¹ is not formula (II): where X, R^{3′} and R^{4′} are defined in the claims.

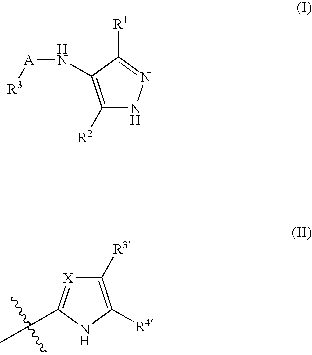

### BACKGROUND OF THE INVENTION

This invention relates to pyrazole compounds that inhibit or modulate the activity of Cyclin Dependent Kinases (CDK), Glycogen Synthase Kinases (GSK) and Aurora kinases to the use of the compounds in the treatment or prophylaxis of disease states or conditions mediated by the kinases, and to novel compounds having kinase inhibitory or modulating activity. Also provided are pharmaceutical compositions containing the compounds and novel chemical intermediates.

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, disease and conditions of the immune system, disease and conditions of the central nervous system, and angiogenesis.

Cyclin Dependent Kinases

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (CDKs) and a diverse set of their cognate protein partners termed cyclins. CDKs are cdc2 (also known as CDK1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific CDK partner proteins.

Modulation of the expression levels, degradation rates, and activation levels of various CDKs and cyclins throughout the cell cycle leads to the cyclical formation of a series of CDK/cyclin complexes, in which the CDKs are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required CDK/cyclin complex, can lead to cell cycle arrest and/or cellular apoptosis. Aberrant cellular proliferation, as manifested in cancer, can often be attributed to loss of correct cell cycle control. Inhibition of CDK enzymatic activity therefore provides a means by which abnormally dividing cells can have their division arrested and/or be killed. The diversity of CDKs, and CDK complexes, and their critical roles in mediating the cell cycle, provides a broad spectrum of potential therapeutic targets selected on the basis of a defined biochemical rationale.

Progression from the G1 phase to the S phase of the cell cycle is primarily regulated by CDK2, CDK3, CDK4 and CDK6 via association with members of the D and E type cyclins. The D-type cyclins appear instrumental in enabling passage beyond the G1 restriction point, where as the CDK2/cyclin E complex is key to the transition from the G1 to S phase. Subsequent progression through S phase and entry into G2 is thought to require the CDK2/cyclin A complex. Both mitosis, and the G2 to M phase transition which triggers it, are regulated by complexes of CDK1 and the A and B type cyclins.

During G1 phase Retinoblastoma protein (Rb), and related pocket proteins such as p130, are substrates for CDK(2, 4, & 6)/cyclin complexes. Progression through G1 is in part facilitated by hyperphosphorylation, and thus inactivation, of Rb and p130 by the CDK(4/6)/cyclin-D complexes. Hyperphosphorylation of Rb and p130 causes the release of transcription factors, such as E2F, and thus the expression of genes necessary for progression through G1 and for entry into S-phase, such as the gene for cyclin E. Expression of cyclin E facilitates formation of the CDK2/cyclin E complex which amplifies, or maintains, E2F levels via further phosphorylation of Rb. The CDK2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the CDK2/cyclin E pathway. CDK2 is also connected to the p53 mediated DNA damage response pathway via p53 regulation of p21 levels. p21 is a protein inhibitor of CDK2/cyclin E and is thus capable of blocking, or delaying, the G1/S transition. The CDK2/cyclin E complex may thus represent a point at which biochemical stimuli from the Rb, Myc and p53 pathways are to some degree integrated. CDK2 and/or the CDK2/cyclin E complex therefore represent good targets for therapeutics designed at arresting, or recovering control of, the cell cycle in aberrantly dividing cells.

The exact role of CDK3 in the cell cycle is not clear. As yet no cognate cyclin partner has been identified, but a dominant negative form of CDK3 delayed cells in G1, thereby suggesting that CDK3 has a role in regulating the G1/S transition.

Although most CDKs have been implicated in regulation of the cell cycle there is evidence that certain members of the CDK family are involved in other biochemical processes. This is exemplified by CDK5 which is necessary for correct neuronal development and which has also been implicated in the phosphorylation of several neuronal proteins such as Tau, NUDE-1, synapsin1, DARPP32 and the Munc18/Syntaxin1A complex. Neuronal CDK5 is conventionally activated by binding to the p35/p39 proteins. CDK5 activity can, however, be deregulated by the binding of p25, a truncated version of p35. Conversion of p35 to p25, and subsequent deregulation of CDK5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

CDK7 is a nuclear protein that has cdc2 CAK activity and binds to cyclin H. CDK7 has been identified as component of the TFIIH transcriptional complex which has RNA polymerase II C-terminal domain (CTD) activity. This has been associated with the regulation of HIV-1 transcription via a Tat-mediated biochemical pathway. CDK8 binds cyclin C and has been implicated in the phosphorylation of the CTD of RNA polymerase II. Similarly the CDK9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. CDK7, CDK8, CDK9 and the P-TEFb complex are therefore potential targets for anti-viral therapeutics.

At a molecular level mediation of CDK/cyclin complex activity requires a series of stimulatory and inhibitory phosphorylation, or dephosphorylation, events. CDK phosphorylation is performed by a group of CDK activating kinases (CAKs) and/or kinases such as wee1, Myt1 and Mik1. Dephosphorylation is performed by phosphatases such as cdc25(a & c), pp2a, or KAP.

CDK/cyclin complex activity may be further regulated by two families of endogenous cellular proteinaceous inhibitors: the Kip/Cip family, or the INK family. The INK proteins specifically bind CDK4 and CDK6. p16^{ink4} (also known as MTS1) is a potential tumour suppressor gene that is mutated, or deleted, in a large number of primary cancers. The Kip/Cip family contains proteins such as p21^{Cip1,Waf1}, p27^{Kip1} and p57^{kip2}. As discussed previously p21 is induced by p53 and is able to inactivate the CDK2/cyclin(E/A) and CDK4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of CDKs, and their associated proteins, in co-ordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable. CDK inhibitors could conceivably also be used to treat other conditions such as viral infections, autoimmune diseases and neuro-degenerative diseases, amongst others. CDK targeted therapeutics may also provide clinical benefits in the treatment of the previously described diseases when used in combination therapy with either existing, or new, therapeutic agents. CDK targeted anticancer therapies could potentially have advantages over many current antitumour agents as they would not directly interact with DNA and should therefore reduce the risk of secondary tumour development.

Diffuse Large B-Cell Lymphomas (DLBCL)

Cell cycle progression is regulated by the combined action of cyclins, cyclin-dependent kinases (CDKs), and CDK-inhibitors (CDKi), which are negative cell cycle regulators. p27KIP1 is a CDKi key in cell cycle regulation, whose degradation is required for G1/S transition. In spite of the absence of p27KIP1 expression in proliferating lymphocytes, some aggressive B-cell lymphomas have been reported to show an anomalous p27KIP1 staining. An abnormally high expression of p27KIP1 was found in lymphomas of this type. Analysis of the clinical relevance of these findings showed that a high level of p27KIP1 expression in this type of tumour is an adverse prognostic marker, in both univariate and multivariate analysis. These results show that there is abnormal p27KIP1 expression in Diffuse Large B-cell Lymphomas (DLBCL), with adverse clinical significance, suggesting that this anomalous p27KIP1 protein may be rendered non-functional through interaction with other cell cycle regulator proteins. (Br. J. Cancer. 1999 July; 80(9):1427-34. p27KIP1 is abnormally expressed in Diffuse Large B-cell Lymphomas and is associated with an adverse clinical outcome. Saez A, Sanchez E, Sanchez-Beato M, Cruz M A, Chacon I, Munoz E, Camacho F I, Martinez-Montero J C, Mollejo M, Garcia J F, Piris M A. Department of Pathology, Virgen de la Salud Hospital, Toledo, Spain.)

Chronic Lymphocytic Leukemia

B-Cell chronic lymphocytic leukaemia (CLL) is the most common leukaemia in the Western hemisphere, with approximately 10,000 new cases diagnosed each year (Parker S L, Tong T, Bolden S, Wingo P A: Cancer statistics, 1997. Ca. Cancer. J. Clin. 47:5, (1997)). Relative to other forms of leukaemia, the overall prognosis of CLL is good, with even the most advanced stage patients having a median survival of 3 years.

The addition of fludarabine as initial therapy for symptomatic CLL patients has led to a higher rate of complete responses (27% v 3%) and duration of progression-free survival (33 v 17 months) as compared with previously used alkylator-based therapies. Although attaining a complete clinical response after therapy is the initial step toward improving survival in CLL, the majority of patients either do not attain complete remission or fail to respond to fludarabine. Furthermore, all patients with CLL treated with fludarabine eventually relapse, making its role as a single agent purely palliative (Rai K R, Peterson B, Elias L, Shepherd L, Hines J, Nelson D, Cheson B, Kolitz J, Schiffer C A: A randomized comparison of fludarabine and chlorambucil for patients with previously untreated chronic lymphocytic leukemia. A CALGB SWOG, CTG/NCI-C and ECOG Inter-Group Study. Blood 88:141a, 1996 (abstr 552, suppl 1). Therefore, identifying new agents with novel mechanisms of action that complement fludarabine's cytotoxicity and abrogate the resistance induced by intrinsic CLL drug-resistance factors will be necessary if further advances in the therapy of this disease are to be realized.

The most extensively studied, uniformly predictive factor for poor response to therapy and inferior survival in CLL patients is aberrant p53 function, as characterized by point mutations or chromosome 17p13 deletions. Indeed, virtually no responses to either alkylator or purine analog therapy have been documented in multiple single institution case series for those CLL patients with abnormal p53 function. Introduction of a therapeutic agent that has the ability to overcome the drug resistance associated with p53 mutation in CLL would potentially be a major advance for the treatment of the disease.

Flavopiridol and CYC 202, inhibitors of cyclin-dependent kinases induce in vitro apoptosis of malignant cells from B-cell chronic lymphocytic leukemia (B-CLL).

Flavopiridol exposure results in the stimulation of caspase 3 activity and in caspase-dependent cleavage of p27(kip1), a negative regulator of the cell cycle, which is overexpressed in B-CLL (Blood. 1998 Nov. 15; 92(10):3804-16 Flavopiridol induces apoptosis in chronic lymphocytic leukemia cells via activation of caspase-3 without evidence of bcl-2 modulation or dependence on functional p53. Byrd J C, Shinn C, Waselenko J K, Fuchs E J, Lehman T A, Nguyen P L, Flinn I W, Diehl L F, Sausville E, Graver M R).

Aurora Kinases

Relatively recently, a new family of serine/threonine kinases known as the Aurora kinases has been discovered that are involved in the G2 and M phases of the cell cycle, and which are important regulators of mitosis.

The precise role of Aurora kinases has yet to be elucidated but that they play a part in mitotic checkpoint control, chromosome dynamics and cytokinesis (Adams et al., Trends Cell Biol., 11: 49-54 (2001). Aurora kinases are located at the centrosomes of interphase cells, at the poles of the bipolar spindle and in the mid-body of the mitotic apparatus.

Three members of the Aurora kinase family have been found in mammals so far (E. A. Nigg, Nat. Rev. Mol. Cell Biol. 2: 21-32, (2001)). These are:

Aurora A (also referred to in the literature as Aurora 2);

Aurora B (also referred to in the literature as Aurora 1); and

Aurora C (also referred to in the literature as Aurora 3).

The Aurora kinases have highly homologous catalytic domains but differ considerably in their N-terminal portions (Katayama H, Brinkley W R, Sen S.; The Aurora kinases: role in cell transformation and tumorigenesis; Cancer Metastasis Rev. 2003 December; 22(4):451-64).

The substrates of the Aurora kinases A and B have been identified as including a kinesin-like motor protein, spindle apparatus proteins, histone H3 protein, kinetochore protein and the tumour suppressor protein p53.

Aurora A kinases are believed to be involved in spindle formation and become localised on the centrosome during the early G2 phase where they phosphorylate spindle-associated proteins (Prigent et al., Cell, 114: 531-535 (2003). Hirota et al, Cell, 114:585-598, (2003) found that cells depleted of Aurora A protein kinase were unable to enter mitosis. Furthermore, it has been found (Adams, 2001) that mutation or disruption of the Aurora A gene in various species leads to mitotic abnormalities, including centrosome separation and maturation defects, spindle aberrations and chromosome segregation defects.

The Aurora kinases are generally expressed at a low level in the majority of normal tissues, the exceptions being tissues with a high proportion of dividing cells such as the thymus and testis. However, elevated levels of Aurora kinases have been found in many human cancers (Giet et al., J. Cell. Sci.112: 3591-361, (1999) and Katayama (2003). Furthermore, Aurora A kinase maps to the chromosome 20q13 region that has frequently been found to be amplified in many human cancers.

Thus, for example, significant Aurora A over-expression has been detected in human breast, ovarian and pancreatic cancers (see Zhou et al., Nat. Genet. 20: 189-193, (1998), Tanaka et al., Cancer Res., 59: 2041-2044, (1999) and Han et al., cancer Res., 62: 2890-2896, (2002).

Moreover, Isola, American Journal of Pathology 147, 905-911 (1995) has reported that amplification of the Aurora A locus (20q13) correlates with poor prognosis for patients with node-negative breast cancer.

Amplification and/or over-expression of Aurora-A is observed in human bladder cancers and amplification of Aurora-A is associated with aneuploidy and aggressive clinical behaviour, see Sen et al., J. Natl. Cancer Inst, 94: 1320-1329 (2002).

Elevated expression of Aurora-A has been detected in over 50% of colorectal cancers, (see Bischoff et al., EMBO J., 17: 3052-3065, (1998) and Takahashi et al., Jpn. J. Cancer Res., 91: 1007-1014 (2000)) ovarian cancers (see Gritsko et al. Clin. Cancer Res., 9: 1420-1426 (2003), and gastric tumours Sakakura et al., British Journal of Cancer, 84: 824-831 (2001).

Tanaka et al. Cancer Research, 59: 2041-2044 (1999) found evidence of over-expression of Aurora A in 94% of invasive duct adenocarcinomas of the breast.

High levels of Aurora A kinase have also been found in renal, cervical, neuroblastoma, melanoma, lymphoma, pancreatic and prostate tumour cell lines Bischoff et al. (1998), EMBO J., 17: 3052-3065 (1998) ; Kimura et al. J. Biol. Chem., 274: 7334-7340 (1999); Zhou et al., Nature Genetics, 20: 189-193 (1998); Li et al., Clin Cancer Res. 9 (3): 991-7 (2003)].

Aurora-B is highly expressed in multiple human tumour cell lines, including leukemic cells [Katayama et al., Gene 244: 1-7) ]. Levels of this enzyme increase as a function of Duke's stage in primary colorectal cancers [Katayama et al., J. Natl Cancer Inst., 91: 1160-1162 (1999)].

High levels of Aurora-3 (Aurora-C) have been detected in several tumour cell lines, even though this kinase tends to be restricted to germ cells in normal tissues (see Kimura et al. Journal of Biological Chemistry, 274: 7334-7340 (1999)). Over-expression of Aurora-3 in approximately 50% of colorectal cancers has also been reported in the article by Takahashi et al., Jpn J. Cancer Res. 91: 1007-1014 (2001)].

Other reports of the role of Aurora kinases in proliferative disorders may be found in Bischoff et al., Trends in Cell Biology 9: 454-459 (1999); Giet et al. Journal of Cell Science, 112: 3591-3601 (1999) and Dutertre, et al. Oncogene, 21: 6175-6183 (2002).

Royce et al report that the expression of the Aurora 2 gene (known as STK15 or BTAK) has been noted in approximately one-fourth of primary breast tumours. (Royce M E, Xia W, Sahin A A, Katayama H, Johnston D A, Hortobagyi G, Sen S, Hung M C; STK15/Aurora-A expression in primary breast tumours is correlated with nuclear grade but not with prognosis; Cancer. 2004 Jan. 1; 100(1):12-9).

Endometrial carcinoma (EC) comprises at least two types of cancer: endometrioid carcinomas (EECs) are estrogen-related tumours, which are frequently euploid and have a good prognosis. Nonendometrioid carcinomas (NEECs; serous and clear cell forms) are not estrogen related, are frequently aneuploid, and are clinically aggressive. It has also been found that Aurora was amplified in 55.5% of NEECs but not in any EECs (P<or=0.001) (Moreno-Bueno G, Sanchez-Estevez C, Cassia R, Rodriguez-Perales S, Diaz-Uriarte R, Dominguez O, Hardisson D, Andujar M, Prat J, Matias-Guiu X, Cigudosa J C, Palacios J. Cancer Res. 2003 Sep. 15; 63(18):5697-702).

Reichardt et al (Oncol Rep. 2003 September-October; 10(5):1275-9) have reported that quantitative DNA analysis by PCR to search for Aurora amplification in gliomas revealed that five out of 16 tumours (31%) of different WHO grade (1× grade II, 1× grade III, 3× grade IV) showed DNA amplification of the Aurora 2 gene. It was hypothesized that amplification of the Aurora 2 gene may be a non-random genetic alteration in human gliomas playing a role in the genetic pathways of tumourigenesis.

Results by Hamada et al (Br. J. Haematol. 2003 May; 121(3):439-47) also suggest that Aurora 2 is an effective candidate to indicate not only disease activity but also tumourigenesis of non-Hodgkin's lymphoma. Retardation of tumour cell growth resulting from the restriction of this gene's functions could be a therapeutic approach for non-Hodgkin's lymphoma.

In a study by Gritsko et al (Clin Cancer Res. 2003 April; 9(4):1420-6)), the kinase activity and protein levels of Aurora A were examined in 92 patients with primary ovarian tumours. In vitro kinase analyses revealed elevated Aurora A kinase activity in 44 cases (48%). Increased Aurora A protein levels were detected in 52 (57%) specimens. High protein levels of Aurora A correlated well with elevated kinase activity.

Results obtained by Li et al (Clin. Cancer Res. 2003 Mar.; 9(3):991-7) showed that the Aurora A gene is overexpressed in pancreatic tumours and carcinoma cell lines and suggest that overexpression of Aurora A may play a role in pancreatic carcinogenesis.

Similarly, it has been shown that Aurora A gene amplification and associated increased expression of the mitotic kinase it encodes are associated with aneuploidy and aggressive clinical behaviour in human bladder cancer. (J. Natl. Cancer Inst. 2002 Sep. 4; 94(17):1320-9).

Investigation by several groups (Dutertre S, Prigent C., Aurora-A overexpression leads to override of the microtubule-kinetochore attachment checkpoint; Mol. Interv. 2003 May; 3(3):127-30 and Anand S, Penrhyn-Lowe S, Venkitaraman A R., Aurora-A amplification overrides the mitotic spindle assembly checkpoint, inducing resistance to Taxol, Cancer Cell. 2003 January; 3(1):51-62) suggests that overexpression of Aurora kinase activity is associated with resistance to some current cancer therapies. For example overexpression of Aurora A in mouse embryo fibroblasts can reduce the sensitivity of these cells to the cytotoxic effects of taxane derivatives. Therefore Aurora kinase inhibitors may find particular use in patients who have developed reistance to existing therapies.

On the basis of work carried out to date, it is envisaged that inhibition of Aurora kinases, particularly Aurora kinase A and Aurora kinase B, will prove an effective means of arresting tumour development.

Harrington et al (Nat Med. 2004 March; 10(3):262-7) have demonstrated that an inhibitor of the Aurora kinases suppresses tumour growth and induces tumour regression in vivo. In the study, the Aurora kinase inhibitor blocked cancer cell proliferation, and also triggered cell death in a range of cancer cell lines including leukaemic, colorectal and breast cell lines. In addition, it has shown potential for the treatment of leukemia by inducing apoptosis in leukemia cells. VX-680 potently killed treatment-refractory primary Acute Myelogenous Leukemia (AML) cells from patients (Andrews, Oncogene, 2005, 24, 5005-5015).

Cancers which may be particularly amenable to Aurora inhibitors include breast, bladder, colorectal, pancreatic, ovarian, non-Hodgkin's lymphoma, gliomas and nonendometrioid endometrial carcinomas. Leukemias particularly amenable to Aurora inhibitors include Acute Myelogenous Leukemia (AML), chronic myelogenous leukaemia (CML), B-cell lymphoma (Mantle cell), and Acute Lymphoblastic Leukemia (ALL).

Glycogen Synthase Kinase

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X—X—X-(pSer/pThr), where X is any amino acid (at positions (n+1), (n+2), (n+3)) and pSer and pThr are phospho-serine and phospho-threonine respectively (n+4). GSK3 phosphorylates the first serine, or threonine, at position (n). Phospho-serine, or phospho-threonine, at the (n+4) position appear necessary for priming GSK3 to give maximal substrate turnover. Phosphorylation of GSK3α at Ser21, or GSK3β at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mammalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus (NIDDM): a condition in which body tissues become resistant to insulin stimulation. The cellular insulin response in liver, adipose, or muscle tissues, is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PI3K) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or ser21, respectively. The inhibition of GSK3 then triggers upregulation of glycogen synthase activity. Therapeutic agents able to inhibit GSK3 may thus be able to induce cellular responses akin to those seen on insulin stimulation. A further in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimulii.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimulii. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours. β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein α (C/EBPα), c-Myc and/or other substrates such as Nuclear Factor of Activated T-cells (NFATc), Heat Shock Factor-1 (HSF-1) and the c-AMP response element binding protein (CREB). GSK3 also appears to play a role, albeit tissue specific, in regulating cellular apoptosis. The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtubule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhibition of Tau phosphorylation, through inhibition of GSK3, may thus provide a means of limiting and/or preventing neurodegenerative effects.

Prior Art

WO 02/34721 from Du Pont discloses a class of indeno [1,2-c]pyrazol-4-ones as inhibitors of cyclin dependent kinases.

WO 01/81348 from Bristol Myers Squibb describes the use of 5-thio-, sulphinyl- and sulphonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (CDKs) and hence their use in the treatment of proliferative disorders such as cancer, leukaemia, psoriasis and the like.

WO 99/21845 from Agouron describes 4-aminothiazole derivatives for inhibiting cyclin-dependent kinases (CDKs), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group.

WO 01/98290 (Pharmacia & Upjohn) discloses a class of 3-aminocarbonyl-2-carboxamido thiophene derivatives as protein kinase inhibitors.

WO 01/53268 and WO 01/02369 from Agouron disclose compounds that mediate or inhibit cell proliferation through the inhibition of protein kinases such as cyclin dependent kinase or tyrosine kinase.

WO 00/39108 and WO 02/00651 (both to Du Pont Pharmaceuticals) describe heterocyclic compounds that are inhibitors of trypsin-like serine protease enzymes, especially factor Xa and thrombin. The compounds are stated to be useful as anticoagulants or for the prevention of thromboembolic disorders.

WO 99/32454, WO 98/28269, WO 98/57937 and WO 98/57951 (all Du Pont Pharmaceuticals) also describe heterocyclic compounds that are inhibitors of factor Xa.

US 2002/0091116 (Zhu et al.), WO 01/19798 and WO 01/64642 each disclose diverse groups of heterocyclic compounds as inhibitors of Factor Xa.

WO 2004/002477 (Merck) discloses 2-phenyl-pyrazole carboxamides as factor Xa inhibitors.

WO 03/035065 (Aventis) discloses a broad class of benzimidazole derivatives as protein kinase inhibitors but does not disclose activity against CDK kinases or GSK kinases.

WO 97/36585, WO 97/36897 and U.S. Pat. No. 5,874,452 (all to Merck) disclose biheteroaryl compounds that are inhibitors of farnesyl transferase.

WO 03/066629 (Vertex) discloses benzimidazolylpyrazole amines as GSK-3 inhibitors.

WO 97/12615 (Warner Lambert) discloses benzimidazoles as 15-lipoxygenase inhibitors.

WO 02/48137 (Du Pont) discloses substituted heterocyclic compounds.

WO 02/024656 (Nihon Nohyaku) discloses pyrazole amides as agricultural agents.

WO 01/56993 (Vertex) discloses pyrazole compounds that have ERK kinase inhibitory activity and which are useful in treating diseases such as cancer, inflammatory disorders, restenosis and cardiovascular disease.

The proviso (i) in formula (I) refers to the disclosures of our earlier International patent applications number PCT/GB2004/002913 and PCT/GB2004/002824 which disclose classes of substituted 1H-benzimidazol-2-yl-1H-pyrazol-4-yl and substituted imidazol-2-yl-1H-pyrazol-4-yl compounds as CDK, Aurora kinase and GSK kinase inhibitors.

The proviso (ii) in formula (I) refers to the disclosures in J. Chem. Research (S), 1977, 140-141.

### SUMMARY OF THE INVENTION

The compounds of the invention are compounds of the general formula (I):

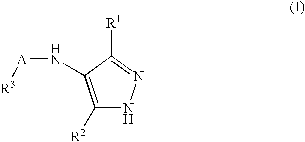

or salts or tautomers or N-oxides or solvates thereof;
wherein
R¹ is an optionally substituted heterocyclic group having from 3 to 12 ring members provided that the cyclic group joined to the pyrazole contains at least one heteroatom selected from N, O or S;
A is a bond or —Y—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
n is 0 or 1;
Y is a bond or an alkylene chain of 1, 2 or 3 carbon atoms in length;
R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);
R³ is selected from optionally substituted carbocyclic and heterocyclic groups having from 3 to 12 ring members or an optionally substituted C₁₋₈ hydrocarbyl group;

Any one or more of the following optional provisos, in any combination, may apply to the compounds of formula (I) and sub-groups thereof:

(i) Where R¹ is not:

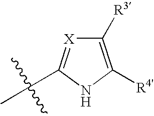

wherein
X is CR^{5′} or N;
R^{3′} and R^{4′} are the same or different and each is selected from hydrogen, CN, C(O)R^{8′}, optionally substituted C₁₋₈ hydrocarbyl and carbocyclic or heterocyclic groups having from 3 to 12 ring members; or
R^{3′} and R^{4′} together with the carbon atoms to which they are attached form an optionally substituted fused carbocyclic or heterocyclic ring having from 5 to 7 ring members of which up to 3 can be heteroatoms selected from N, O and S; and
R^{5′} is hydrogen, a group R^{2′} or a group R^{10′} wherein R^{10′} is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a′}-R^{b′} wherein R^{a′} is a bond, O, CO, X^{1′}C(X^{2′}), C(X^{2′})X^{1′}, X^{1′}C(X^{2′})X^{1′}, S, SO, SO₂, SO₂NR^{c′} or NR^{c′}SO₂; and R^{b′} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c′}, X^{1′}C(X^{2′}), C(X^{2′})X^{1′} or X^{1′}C(X^{2′})X^{1′};
R^{2′} is hydrogen, halogen, methoxy, or a C₁₋₄ hydrocarbyl group optionally substituted by halogen, hydroxyl or methoxy;
R^{c′} is selected from hydrogen and C₁₋₄ hydrocarbyl; and
X^{1′} is O, S or NR^{c′} and X^{2′} is ═O, ═S or ═NR^{c′};
R^{8′} is selected from OR^{11′}, SR^{11′} and NR^{12′}R^{13′};
R^{11′} is selected from optionally substituted C₁₋₈ hydrocarbyl and carbocyclic or heterocyclic groups having from 3 to 12 ring members; and one of R^{12′} and R^{13′} is a group R^{11′} and the other of R^{12′} and R^{13′} is hydrogen or C₁₋₄ alkyl; or R^{12′} and R^{13′} and the nitrogen atom to which they are attached together form a saturated heterocyclic group having from 4 to 7 ring members and containing 1, 2 or 3 heteroatom ring members selected from N, O and S.
   and salts, N-oxides and solvates thereof.

(ii) The compound of formula (I) is not N-[3-(morpholin-4-yl)-5-(trifluoromethyl)-1H-pyrazol-4-yl]-benzamide, 2,2,2-trifluoro-N-(3-morpholin-4-yl-5-trifluoromethyl-1H-pyrazol-4-yl)-acetamide and 4-nitro-N-[3-(piperidin-1-yl)-5-(trifluoromethyl)-1H-pyrazol-4-yl]-benzamide.

The invention provides compounds that have cyclin dependent kinase inhibiting or modulating activity and glycogen synthase kinase-3 (GSK3) inhibiting or modulating activity, or Aurora kinase inhibiting or modulating activity, and which it is envisaged will be useful in preventing or treating disease states or conditions mediated by these kinases.

Thus, for example, it is envisaged that the compounds of the invention will be useful in alleviating or reducing the incidence of cancer.

Accordingly, the invention provides inter alia:
A pharmaceutical composition comprising a compound of the formula (I) and sub-groups thereof as defined herein and a pharmaceutically acceptable carrier.
Compounds of the formula (I) and sub-groups thereof as defined herein for use in medicine.
The use of a compound of the formula (I) and sub-groups thereof as defined herein, for the prophylaxis or treatment of any one of the disease states or conditions disclosed herein.
The use of a compound of the formula (I) and sub-groups thereof as defined herein, for the prophylaxis or treatment of a disease state or condition mediated by a cyclin dependent kinase or glycogen synthase kinase-3.
The use of a compound of the formula (I) and sub-groups thereof as defined herein, for prophylaxis or treatment of a disease or condition characterised by up-regulation of an Aurora kinase (e.g. Aurora A kinase or Aurora B kinase).
The use of a compound of the formula (I) and sub-groups thereof as defined herein, for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I) and sub-groups thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I) and sub-groups and sub-groups thereof as defined herein.
The use of a compound of the formula (I) and sub-groups as defined herein for the manufacture of a medicament for the prophylaxis or treatment of a disease state or condition mediated by a cyclin dependent kinase or glycogen synthase kinase-3.
A method for the prophylaxis or treatment of a disease state or condition mediated by a cyclin dependent kinase or glycogen synthase kinase-3, which method comprises administering to a subject in need thereof a compound of the formula (I) and sub-groups as defined herein.
A method for alleviating or reducing the incidence of a disease state or condition mediated by a cyclin dependent kinase or glycogen synthase kinase-3, which method comprises administering to a subject in need thereof a compound of the formula (I) and sub-groups as defined herein.
A method for treating a disease or condition comprising or arising from abnormal cell growth in a mammal, which method comprises administering to the mammal a compound of the formula (I) and sub-groups as defined herein in an amount effective in inhibiting abnormal cell growth.
A method for alleviating or reducing the incidence of a disease or condition comprising or arising from abnormal cell growth in a mammal, which method comprises administering to the mammal a compound of the formula (I) and sub-groups as defined herein in an amount effective in inhibiting abnormal cell growth.
A method for treating a disease or condition comprising or arising from abnormal cell growth in a mammal, the method comprising administering to the mammal a compound of the formula (I) and sub-groups as defined herein in an amount effective to inhibit a CDK kinase (such as CDK1 or CDK2) or glycogen synthase kinase-3 activity.
A method for alleviating or reducing the incidence of a disease or condition comprising or arising from abnormal cell growth in a mammal, the method comprising administering to the mammal a compound of the formula (I) and sub-groups as defined herein in an amount effective to inhibit a CDK kinase (such as CDK1 or CDK2) or glycogen synthase kinase-3 activity.
A method of inhibiting a cyclin dependent kinase or glycogen synthase kinase-3, which method comprises contacting the kinase with a kinase-inhibiting compound of the formula (I) and sub-groups as defined herein.
A method of modulating a cellular process (for example cell division) by inhibiting the activity of a cyclin dependent kinase or glycogen synthase kinase-3 using a compound of the formula (I) and sub-groups as defined herein.
A method for the diagnosis and treatment of a disease state or condition mediated by a cyclin dependent kinase, which method comprises (i) screening a patient to determine whether a disease or condition from which the patient is or may be suffering is one which would be susceptible to treatment with a compound having activity against cyclin dependent kinases; and (ii) where it is indicated that the disease or condition from which the patient is thus susceptible, thereafter administering to the patient a compound of the formula (I) and sub-groups thereof as defined herein.
The use of a compound of the formula (I) and sub-groups thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against cyclin dependent kinase.
The use of a compound of the formula (I) as defined herein for the manufacture of a medicament for prophylaxis or treatment of a disease or condition characterised by up-regulation of an Aurora kinase (e.g. Aurora A kinase or Aurora B kinase).
The use of a compound of the formula (I) as defined herein for the manufacture of a medicament for the prophylaxis or treatment of a cancer, the cancer being one which is characterised by up-regulation of an Aurora kinase (e.g. Aurora A kinase or Aurora B kinase).
The use of a compound of the formula (I) as defined herein for the manufacture of a medicament for the prophylaxis or treatment of cancer in a patient selected from a sub-population possessing the Ile31 variant of the Aurora A gene.
The use of a compound of the formula (I) as defined herein for the manufacture of a medicament for the prophylaxis or treatment of cancer in a patient who has been diagnosed as forming part of a sub-population possessing the Ile31 variant of the Aurora A gene.
A method for the prophylaxis or treatment of a disease or condition characterised by up-regulation of an Aurora kinase (e.g. Aurora A kinase or Aurora B kinase), the method comprising administering a compound of the formula (I) as defined herein.
A method for alleviating or reducing the incidence of a disease or condition characterised by up-regulation of an Aurora kinase (e.g. Aurora A kinase or Aurora B kinase), the method comprising administering a compound of the formula (I) as defined herein.
A method for the prophylaxis or treatment of (or alleviating or reducing the incidence of) cancer in a patient suffering from or suspected of suffering from cancer; which method comprises (i) subjecting a patient to a diagnostic test to determine whether the patient possesses the Ile31 variant of the Aurora A gene; and (ii) where the patient does possess the said variant, thereafter administering to the patient a compound of the formula (I) as defined herein having Aurora kinase inhibiting activity.
A method for the prophylaxis or treatment of (or alleviating or reducing the incidence of) a disease state or condition characterised by up-regulation of an Aurora kinase (e.g. Aurora A kinase or Aurora B kinase); which method comprises (i) subjecting a patient to a diagnostic test to detect a marker characteristic of up-regulation of the Aurora kinase and (ii) where the diagnostic test is indicative of up-regulation of Aurora kinase, thereafter administering to the patient a compound of the formula (I) as defined herein having Aurora kinase inhibiting activity.
The use of a compound of the formula (I) as defined herein for the manufacture of a medicament for the prophylaxis or treatment of a disease state as described herein.
A compound of the formula (I) as defined herein for use in the prophylaxis or treatment of a disease state as described herein.
A compound of formula (I) or a salt (e.g. an acid addition salt), solvate, tautomer or N-oxide thereof for use in the treatment of B-cell lymphoma.
A compound of formula (I) or a salt (e.g. an acid addition salt), solvate, tautomer or N-oxide thereof for use in the treatment of chronic lymphocytic leukaemia.
A compound of formula (I) or a salt (e.g. an acid addition salt), solvate, tautomer or N-oxide thereof for use in the treatment of diffuse large B cell lymphoma.
A method of treatment of B-cell lymphoma, diffuse large B cell lymphoma or chronic lymphocytic leukaemia by administering to a patient in need of such treatment a compound of formula (I) or a salt (e.g. an acid addition salt), solvate, tautomer or N-oxide thereof.
A compound of formula (I) or a salt (e.g. an acid addition salt), solvate, tautomer or N-oxide thereof for use in the treatment of leukaemia in particular relapsed or refractory acute myelogenous leukemia, myelodysplastic syndrome, acute lymphocytic leukemia and chronic myelogenous leukemia.

The aforementioned methods and uses, and any other therapeutic and diagnostic methods and uses, and methods of treating animals and plants defined herein, may also employ any sub-group, sub-genus, preference or example falling within formula (I), for example the compounds of formulae (II) and (III) and any sub-groups thereof, unless the context indicates otherwise.

In each of the foregoing uses, methods and other aspects of the invention, as well as any aspects and embodiments of the invention as set out below, references to compounds of the formula (I) and sub-groups thereof as defined herein include within their scope the salts or solvates or tautomers or N-oxides of the compounds.

General Preferences and Definitions

The following general preferences and definitions shall apply to each of the moieties A, B, Y, R^{g}, R¹ to R³ and any sub-definition, sub-group or embodiment thereof, unless the context indicates otherwise.

In this specification, references to formula (I) include formulae (II) and (III) and sub-groups, examples or embodiments of formulae (II) and (III) unless the context indicates otherwise.

Thus for example, references to inter alia therapeutic uses, pharmaceutical formulations and processes for making compounds, where they refer to formula (I), are also to be taken as referring to formulae (II) and (III) and sub-groups, examples or embodiments of formulae (II) and (III).

Similarly, where preferences, embodiments and examples are given for compounds of the formula (I), they are also applicable to formulae (II) and (III) and sub-groups, examples or embodiments of formulae (II) and (III) unless the context requires otherwise.

References to “carbocyclic” and “heterocyclic” groups as used herein shall, unless the context indicates otherwise, include both aromatic and non-aromatic ring systems. Thus, for example, the term “carbocyclic and heterocyclic groups” includes within its scope aromatic, non-aromatic, unsaturated, partially saturated and fully saturated carbocyclic and heterocyclic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 3 or 6 in particular 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character, that is groups with conjugated bonds. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term “non-aromatic group” embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C═C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl. A further example of a cycloalkenyl group is cyclohexenyl. Saturated heterocyclic groups include piperidine, morpholine, thiomorpholine. Partially saturated heterocyclic groups include pyrazolines, for example 2-pyrazoline and 3-pyrazoline.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazol-4-yl, furazan, oxazole, oxadiazole, oxatriazole, isoxazole, thiazole, isothiazole, pyrazole, triazole, dihydro-triazole-thione e.g. 2,4-dihydro-[1,2,4]triazole-3-thione, dihydro-triazol-one e.g. 2,4-dihydro-[1,2,4]triazol-3-one and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

One sub-group of bicyclic heteroaryl groups consists of groups (a) to (e) and (g) to (o) above.

Particular examples of bicyclic heteroaryl groups containing a five membered ring fused to another five membered ring include but are not limited to pyrazolopyrazole e.g. pyrazolo[3,4-c]pyrazole, imidazothiazole (e.g. imidazo[5,1-b]thiazole) and imidazoimidazole (e.g. imidazo[1,5-c]imidazole) but do not comprise a fused imidazol-2-yl.

Particular examples of bicyclic heteroaryl groups containing a six membered ring fused to a five membered ring include but are not limited to benzfuran, benzthiophene, benzoxazole, isobenzoxazole, benzisoxazole, benzthiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), triazolopyrimidine (e.g. [1,2,4]triazolo[1,5-a]pyrimidine), benzodioxole, imidazo[1,2-a]pyridine (e.g. 3-imidazo[1,2-a]pyridin-2-yl) and pyrazolopyridine (e.g. pyrazolo[1,5-a]pyridine) groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

One sub-group of heteroaryl groups comprises pyridyl, pyrrolyl, furanyl, thienyl, imidazol-4-yl, oxazolyl, oxadiazolyl, oxatriazolyl, isoxazolyl, thiazolyl, isothiazolyl, pyrazolyl, pyrazinyl, pyridazinyl, pyrimidinyl, triazinyl, triazolyl, tetrazolyl, quinolinyl, isoquinolinyl, benzfuranyl, benzthienyl, chromanyl, thiochromanyl, benzoxazolyl, benzisoxazole, benzthiazolyl and benzisothiazole, isobenzofuranyl, indolyl, isoindolyl, indolizinyl, indolinyl, isoindolinyl, purinyl (e.g., adenine, guanine), indazolyl, benzodioxolyl, chromenyl, isochromenyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1,2,3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide). Further examples of heterocyclic groups are those containing a cyclic urea moiety (e.g. as in imidazolidin-2-one),

In one sub-set of heterocyclic groups, the heterocyclic groups contain cyclic ether moieties (e.g as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulpholane and sulpholene), cyclic sulphoxides, cyclic sulphonamides and combinations thereof (e.g. thiomorpholine).

Examples of monocyclic non-aromatic heterocyclic groups include 5-, 6-and 7-membered monocyclic heterocyclic groups. Particular examples include morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine.

One preferred sub-set of non-aromatic heterocyclic groups consists of saturated groups such as azetidine, pyrrolidine, piperidine, morpholine, thiomorpholine, thiomorpholine S,S-dioxide, piperazine, N-alkyl piperazines, and N-alkyl piperidines.

Another sub-set of non-aromatic heterocyclic groups consists of pyrrolidine, piperidine, morpholine, thiomorpholine, thiomorpholine S,S-dioxide, piperazine and N-alkyl piperazines such as N-methyl piperazine.

One particular sub-set of heterocyclic groups consists of pyrrolidine, piperidine, morpholine and N-alkyl piperazines (e.g. N-methyl piperazine), and optionally thiomorpholine.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocyclic groups includes unsubstituted or substituted (by one or more groups R¹⁰) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane. A particular example of a bridged ring system is the 1-aza-bicyclo[2.2.2]octan-3-yl group.

Nitrogen-containing heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur, where the ring must contain at least one ring nitrogen atom. The heterocyclic groups can be non-aromatic or aromatic. In one embodiment it is aromatic. The heterocylic groups can contain, for example cyclic amine moieties (e.g. as in pyrrolidine), cyclic amides (such as a pyrrolidinone, piperidone or caprolactam), cyclic sulphonamides (such as an isothiazolidine 1,1-dioxide, [1,2]thiazinane 1,1-dioxide or [1,2]thiazepane 1,1-dioxide) and combinations thereof.

Examples of nitrogen-containing heteroaryl groups include, but are not limited to, pyridyl, pyrrolyl, imidazolyl, oxazolyl, oxadiazolyl, thiadiazolyl, oxatriazolyl, isoxazolyl, thiazolyl, isothiazolyl, furazanyl, pyrazolyl, pyrazinyl, pyrimidinyl, pyridazinyl, triazinyl, triazolyl (e.g., 1,2,3-triazolyl, 1,2,4-triazolyl), tetrazolyl, quinolinyl, isoquinolinyl, benzimidazolyl, benzoxazolyl, benzisoxazole, benzthiazolyl and benzisothiazole, indolyl, 3H-indolyl, isoindolyl, indolizinyl, isoindolinyl, purinyl (e.g., adenine [6-aminopurine], guanine [2-amino-6-hydroxypurine]), indazolyl, quinolizinyl, benzoxazinyl, benzodiazinyl, pyridopyridinyl, quinoxalinyl, quinazolinyl, cinnolinyl, phthalazinyl, naphthyridinyl and pteridinyl.

Examples of nitrogen-containing polycyclic heteroaryl groups containing an aromatic ring and a non-aromatic ring include tetrahydroisoquinolinyl, tetrahydroquinolinyl, and indolinyl.

Particular examples of non-aromatic nitrogen-containing heterocyclic groups include aziridine, morpholine, thiomorpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, dihydrothiazole, imidazoline, imidazolidinone, oxazoline, thiazoline, 6H-1,2,5-thiadiazine, 2-pyrazoline, 3-pyrazoline, pyrazolidine, piperazine, and N-alkyl piperazines such as N-methyl piperazine.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 11, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. Thus, two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group.

Examples of such linked substituent groups include:

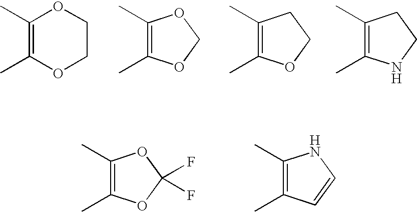

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms.

Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Preferred non-aromatic hydrocarbyl groups are saturated groups such as alkyl and cycloalkyl groups.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl(vinyl), 1-propenyl, 2-propenyl(allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members, more usually 3, 4, 5 or 6 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members, more usually 5 to 6 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O) NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl and trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

Specific Embodiments of and Preferences for A, B, Y, R^{g}, R¹ to R³

A, B, Y and R^{g}

A is a bond or —Y—(B)ₙ— wherein B is C═O, NR^{g}(C═O) or O(C═O), Y is a bond or an alkylene chain of 1, 2 or 3 carbon atoms in length and n is 0 or 1.

It will be appreciated that the moiety R³-A-NH linked to the 4-position of the pyrazole ring can take the form of an amine R³—Y—NH (where n=0), or an amide R³—Y—C(═O)NH, or a urea R³—Y—NHC(═O)NH or a carbamate R³—Y—OC(═O)NH (where n=1) wherein in each case Y is a bond or an alkylene chain of 1, 2 or 3 carbon atoms in length.

Preferably Y is a bond or an alkylene chain of 1 carbon atom in length and most preferably a bond.

In one group of compounds of the invention, A is Y—C═O and hence the group R³-A-NH takes the form of an amide R³—Y—C(═O)NH, in particular Y is a bond and therefore the group is R³—C(═O)NH.

In another group of compounds of the invention, A is —Y—(B)ₙ— and n=0 so that the group takes the form of an amine R³—Y—NH. In particular Y is a bond and hence the group R³-A-NH takes the form of the amine R³—NH.

In a further group of compounds of the invention, A is Y—NR^{g}C(═O) [where A is —Y—(B)ₙ—, n=1 and B is NR^{g}C(═O)] and hence the group R³-A-NH takes the form of an urea R³—Y—NR^{g}C(═O)NH, in particular Y is a bond and therefore the group is R³—NR^{g}C(═O)NH.

In one preferred group of compounds of the invention, A is —Y—(B)ₙ, n is 1 and B is C═O or NR^{g}(C═O). More preferably n is 1 and B is C═O.

It is presently preferred that when B is NR^{g}(C═O), R^{g} is hydrogen.

R¹

In one embodiment, R¹ is a heteroaryl.

In another embodiment, R¹ is a nitrogen containing heterocyclic group having from 3 to 12 ring members where the cyclic group joined to the pyrazole contains at least one heteroatom.

In another embodiment, R¹ is a nitrogen containing heteroaryl group having from 3 to 12 ring members provided that the cyclic group joined to the pyrazole contains at least one heteroatom.

In another embodiment, R¹ is a monocyclic or bicyclic heterocyclic group having from 3 to 12 ring members where the cyclic group joined to the pyrazole contains at least one heteroatom.

In further embodiment, R¹ is a monocyclic heterocyclic group having from 3 to 7 ring members.

In further embodiment, R¹ is a monocyclic heterocyclic group having from 5 to 6 ring members.

In further embodiment, R¹ is a monocyclic heteroaryl group having from 5 to 6 ring members.

In another embodiment, R¹ is a monocyclic nitrogen containing heteroaryl group having from 5 to 6 ring members.

In one embodiment R¹ is a five membered heteroaryl groups selected from pyrrole, furan, thiophene, imidazol-4-yl, furazan, oxazole, oxadiazole, isoxazole, thiazole, isothiazole, pyrazole, 2,4-dihydro-[1,2,4]triazole-3-thione, 2,4-dihydro-[1,2,4]triazol-3-one, and triazole groups.

In one embodiment R¹ is a substituted five membered heteroaryl group.

In another embodiment where R¹ is a substituted five membered heteroaryl group the one or more substituents are attached to ring nitrogens, in particular the substituted ring nitrogen is not adjacent to the atom joined to the pyrazole moiety. In one embodiment R¹ is a 2- substituted -2,4-dihydro-[1,2,4]triazol-3-one.

In another embodiment, R¹ is unsubstituted or substituted oxazole in particular oxazol-4-yl.

In one embodiment R¹ is a six membered heteroaryl groups selected from pyridine, pyrazine, pyridazine, pyrimidine and triazine.

In one embodiment, R¹ is unsubstituted or substituted pyridine, pyrazine, or oxazole.

In one embodiment, R¹ is unsubstituted or substituted pyridine such as 2-pyridinyl, 3-pyridinyl, 4-pyridinyl in particular 2-pyridinyl.

In one embodiment, R¹ is unsubstituted or substituted pyrazine in particular 1,4-pyrazin-2-yl.

In one embodiment R¹ is not a triazole.

In one embodiment where A is a bond it is preferred that R¹ is not pyrimidine or triazole.

In another embodiment R¹ is not morpholine or piperidine.

In the context of R¹, in one particular embodiment, the heterocyclic group has a ring heteroatom selected from O, S and N (preferably S and N, and more particularly N), the ring heteroatom being positioned adjacent to the atom joined to the pyrazole moiety. In one embodiment when the adjacent ring heteroatom is nitrogen, most preferably the nitrogen atom is unsubstituted.

By way of explanation, in the substructures shown below, when R¹ is a 2-pyridyl group as shown in M, the nitrogen heteroatom is adjacent to the carbon atom joined to the pyrazole moiety yet when R¹ is a 3-pyridyl group, as shown in N, is not.

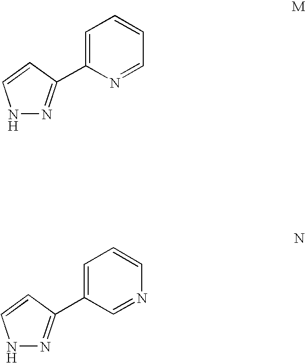

The ring heteroatom can be positioned adjacent to a carbon atom or nitrogen atom which is joined to the pyrazole moiety. In one embodiment the atom joined to the pyrazole moiety is nitrogen. In another embodiment the atom joined to the pyrazole moiety is carbon.

Examples of heterocyclic groups R¹ in which the R¹ ring heteratom is adjacent to the carbon or nitrogen atom joined to the pyrazole moiety include 2-pyridyl (as shown above), 2-thiadiazolyl, 2,5-pyrazinyl, 2-pyrrolidinyl, 2-pyridazinyl, 2,4-dihydro-[1,2,4]triazole-3-thione, 2,4-dihydro-[1,2,4]triazol-3-one, oxazol-2-yl, [1,3,4]oxadiazol-2-yl, isoxazole-3-yl, thiazole-2-yl and 2-quinolinyl groups.

In another embodiment R¹ is a bicyclic group, which typically has 8 to 10 ring members, for example 8, or 9, or 10 ring members, provided that when R¹ is a bicyclic group the ring connected to the pyrazole contains at least one heteroatom selected from N, O or S. The bicyclic group can be an heteroaryl group and examples of such groups include groups comprising a 5-membered ring fused to another 5-membered ring; a 5-membered ring fused to a 6-membered ring; and a 6-membered ring fused to another 6-membered ring. In particular it is a bicyclic heteroaryl group comprising a 5-membered ring fused to a 6-membered ring.

In one embodiment the R¹ group is a bicyclic heteroaryl groups comprising a 5-membered aromatic ring fused a 6-membered aryl ring i.e. phenyl, provided that the R¹ group is joined to the pyrazole via the 5-membered ring.

In another embodiment the R¹ group is a bicyclic heteroaryl groups comprising a 5-membered aromatic ring such as pyrazole or imidazole fused a 6-membered ring, provided that the R¹ group does not comprise a imidazo-2-yl group. Particular examples include pyrazole or imidazole fused to phenyl such as indazole e.g. 3-indazol-2-yl and imidazo[1,2-a]pyridine e.g. 3-imidazo[1,2-a]pyridin-2-yl.

In one embodiment the R¹ group is a bicyclic heteroaryl groups comprising a 5-membered ring non-aromatic fused a 6-membered phenyl ring provided the R¹ group is joined to the pyrazole via the 5-membered ring. In particular the 5-membered ring non-aromatic is pyrrolidine-2,5-dione thereby giving 1,3-dioxo-1,3-dihydro-isoindol-2-yl.

In one embodiment the R¹ group is a bicyclic heteroaryl groups comprising a 5-membered ring fused to 6-membered heteroaryl ring such as pyridine or pyrazine or pyrimidine such as 1H-imidazo[4,5-b]pyridine, or 1H-pyrrolo[3,2-b]pyridine.

A bicyclic heteroaryl group can comprise two aromatic or unsaturated rings, or one aromatic and one non-aromatic (e.g. partially saturated) ring. In particular it comprises two aromatic rings.

Bicyclic heteroaryl R¹ groups typically contain up to 4 heteroatom ring members selected from N, S and O. Thus, for example, they may contain 1, or 2, or 3, or 4 heteroatom ring members.

For the bicyclic heterocyclic R¹ groups, examples of combinations of heteroatom ring members include N; NN; NNN; NNNN; NO; NNO; NS, NNS, O, S, OO and SS. Preferred combinations are N; NN; and NNN in particular N; and NN.

The substituent R¹⁰ on R¹ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

The group R¹ can be an unsubstituted or substituted carbocylic or heterocyclic group in which one or more substituents can be selected from the group R¹⁰ as hereinbefore defined. In one embodiment, the substituents on R¹ may be selected from the group R^{10a} consisting of halogen, hydroxy, trifluoromethyl, carbocyclic and heterocyclic groups having from 3 to 10 ring members and a group R^{a}-R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, and monocyclic non-aromatic carbocyclic or heterocyclic groups having from 3 to 6 ring members; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

The group R¹ can be an unsubstituted or substituted carbocylic or heterocyclic group in which one or more substituents can be selected from the group R¹⁰ as hereinbefore defined. In one embodiment, the substituents on R¹ may be selected from the group R^{10b} consisting of halogen, hydroxy, trifluoromethyl, aromatic carbocyclic and heterocyclic groups having from 3 to 10 ring members and a group R^{a}-R^{b} wherein R^{a} is a bond, O, CO, X³C(X⁴), C(X⁴)X³, X³C(X⁴)X³, and R^{b} is selected from hydrogen and a C₁₋₈ hydrocarbyl group optionally substituted by one or more substituents selected from hydroxy, oxo, halogen, and monocyclic non-aromatic carbocyclic or heterocyclic groups having from 3 to 6 ring members.

In another embodiment the substituents on R¹ may be selected from the group R^{10c} consisting of halogen, hydroxy, trifluoromethyl, aromatic monocyclic carbocyclic and heterocyclic groups having from 5 or 6 ring members and a group R^{a}-R^{b} wherein R^{a} is a bond, O, and R^{b} is selected from hydrogen and a C₁₋₈ alkyl group optionally substituted by one or more substituents selected from hydroxy, oxo, halogen and monocyclic non-aromatic carbocyclic or heterocyclic groups having from 5 to 6 ring members.

More particularly, the substituents on R¹ may be selected from halogen, hydroxy, trifluoromethyl, aromatic monocyclic carbocyclic and heterocyclic groups having from 5 or 6 ring members and a group R^{a}-R^{b} wherein R^{a} is a bond or O, and R^{b} is selected from hydrogen and a C₁₋₄ alkyl group optionally substituted by one or more substituents selected from hydroxyl, halogen (preferably fluorine) and 5 and 6 membered saturated carbocyclic and heterocyclic groups (for example groups containing up to two heteroatoms selected from O, S and N, such as unsubstituted piperidine, pyrrolidino, morpholino, piperazino and N-methyl piperazino).

In one embodiment R¹ is an optionally substituted oxadiazole e.g. [1,3,4]oxadiazole, optionally substituted 2,4-dihydro-[1,2,4]triazole-3-thione, optionally substituted 2,4-dihydro-[1,2,4]triazol-3-one, optionally substituted pyrazole, optionally substituted imidazo[1,2-a]pyridine, optionally substituted thiazole, optionally substituted pyridine, optionally substituted pyrazine, optionally substituted indazole, optionally substituted oxazole, optionally substituted 1H-imidazol-4-yl, and optionally substituted triazole.

In another embodiment R¹ is unsubstitued [1,3,4]oxadiazole, 4-substituted-2,4-dihydro-[1,2,4]triazole-3-thione, unsubstituted 2,4-dihydro-[1,2,4]triazol-3-one; 2-substituted-2,4-dihydro-[1,2,4]triazol-3-one, unsubstituted 1H-pyrazole, 1-substituted pyrazole, 3-substituted pyrazole, 5-substituted pyrazole, unsubstituted imidazo[1,2-a]pyridine, 2-substituted-thiazole, unsubstituted pyridine, unsubstituted pyrazine, unsubstituted indazole, 5-substituted-isoxazole, unsubstituted oxazole, 2-substituted-oxazole, 2-substituted-1H-imidazol-4-yl and 3-substituted-2H-[1,2,4]triazole.

Preferred optional substituents R¹⁰ on R¹ are optionally substituted C₁₋₄ alkyl, or phenyl. In particular C₁₋₄ alkyl, or phenyl substituted with halogen or 5-7 membered unsaturated heterocycle.

In one embodiment the optional substituents on R¹ are C₁₋₄ alkyl, or phenyl substituted with halogen such as fluorine or 6 membered unsaturated heterocycle in particular morpholine.

Preferred R¹⁰ substituents are unsubstituted methyl, unsubstituted propyl such as i-propyl, unsubstituted butyl such as t-butyl, 2-morpholin-4-yl-ethyl, trifluoromethyl, unsubstituted phenyl, and 4-fluorophenyl.

In a further embodiment R¹ is unsubstituted [1,3,4]oxadiazol-2-yl; 4-methyl-2,4-dihydro-[1,2,4]triazole-3-thione in particular 4-methyl-5-thioxo-4,5-dihydro-1H-[1,2,4]triazol-3-yl; unsubstituted 3-(5-oxo-4,5-dihydro-1H-[1,2,4]triazol-3-yl; 2-isopropyl-2,4-dihydro-[1,2,4]triazol-3-one, 2-methyl-2,4-dihydro-[1,2,4]triazol-3-one and N-{3-[1-(2-morpholin-4-yl-ethyl)-5-oxo-4,5-dihydro-1H-[1,2,4]triazole] in particular N-[3-(1-isopropyl-5-oxo-4,5-dihydro-1H-[1,2,4]triazol-3-yl, 1-methyl-5-oxo-4,5-dihydro-1H-[1,2,4]triazol-3-yl, and N-{3-[1-(2-morpholin-4-yl-ethyl)-5-oxo-4,5-dihydro-1H-[1,2,4]triazol-3-yl]; unsubstituted pyrazol-4-yl; unsubstituted 1H-pyrazol-1-yl; 1-tert-butyl-1H-pyrazole, 1-methyl-1H-pyrazole in particular 1-tert-butyl-1H-pyrazol-4-yl and 1-methyl-1H-pyrazol-4-yl; 3-phenyl-1H-pyrazole, 3-(4-fluoro-phenyl)-1H-pyrazole, and 3-trifluoromethyl-1H-pyrazole in particular 3-phenyl-1H-pyrazol-1-yl, 3-(4-fluoro-phenyl)-1H-pyrazol-1-yl, and 3-trifluoromethyl-1H-pyrazol-1-yl; 5-trifluoromethyl-1H-pyrazole in particular 5-trifluoromethyl-1H-pyrazol-1-yl; unsubstituted 3-imidazo[1,2-a]pyridin-2-yl; 2-methyl-thiazole and 2-phenyl-thiazole in particular 2-methyl-thiazol-4-yl, and 2-phenyl-thiazol-4-yl; unsubstituted 2-pyridinyl; unsubstituted 1,4-pyrazin-2-yl; unsubstituted 3-indazol-2-yl; 5-methyl-isoxazole in particular 5-methyl-isoxazol-3-yl; unsubstituted 3-oxazol-5-yl; 2-methyl-oxazole in particular 2-methyl-oxazol-4-yl; 2-trifluoromethyl-1H-imidazol-4-yl and 3-phenyl-2H-[1,2,4]triazole in particular 3-phenyl-2H-[1,2,4]triazol-5-yl.

In one embodiment R¹ is optionally substituted 2,4-dihydro-[1,2,4]triazol-3-one, optionally substituted pyridine, optionally substituted pyrazine, optionally substituted pyrazole, optionally substituted thiazole, optionally substituted imidazo[1,2-a]pyridine, and optionally substituted oxazole.

In another embodiment R¹ is, unsubstituted 2,4-dihydro-[1,2,4]triazol-3-one; 2-substituted-2,4-dihydro-[1,2,4]triazol-3-one, unsubstituted pyridine, unsubstituted pyrazine, unsubstituted 1H-pyrazole, 3-substituted pyrazole, 2-substituted-thiazole, unsubstituted imidazo[1,2-a]pyridine, and 2-substituted-oxazole.

In a further embodiment R¹ is unsubstituted 3-(5-oxo-4,5-dihydro-1H-[1,2,4]triazol-3-yl; 2-isopropyl-2,4-dihydro-[1,2,4]triazol-3-one in particular N-[3-(1-isopropyl-5-oxo-4,5-dihydro-1H-[1,2,4]triazol-3-yl; unsubstituted 1H-pyrazol-1-yl; 3-phenyl-1H-pyrazole, and 3-(4-fluoro-phenyl)-1H-pyrazole in particular 3-phenyl-1H-pyrazol-1-yl, and 3-(4-fluoro-phenyl)-1H-pyrazol-1-yl; unsubstituted 3-imidazo[1,2-a]pyridin-2-yl; 2-methyl-thiazole and 2-phenyl-thiazole in particular 2-methyl-thiazol-4-yl, and 2-phenyl-thiazol-4-yl; unsubstituted 2-pyridinyl; unsubstituted 1,4-pyrazin-2-yl; and 2-methyl-oxazole in particular 2-methyl-oxazol-4-yl.

In one embodiment R¹ is optionally substituted 2,4-dihydro-[1,2,4]triazol-3-one, optionally substituted pyridine, optionally substituted pyrazole, optionally substituted thiazole, optionally substituted imidazo[1,2-a]pyridine, and optionally substituted oxazole.

In another embodiment R¹ is 2-substituted-2,4-dihydro-[1,2,4]triazol-3-one, unsubstituted pyridine, 3-substituted pyrazole, 2-substituted-thiazole, unsubstituted imidazo[1,2-a]pyridine, and 2-substituted-oxazole.

In a further embodiment R¹ is 2-isopropyl-2,4-dihydro-[1,2,4]triazol-3-one in particular N-[3-(1-isopropyl-5-oxo-4,5-dihydro-1H-[1,2,4]triazol-3-yl; 3-phenyl-1H-pyrazole in particular 3-phenyl-1H-pyrazol-1-yl; unsubstituted 3-imidazo[1,2-a]pyridin-2-yl; 2-phenyl-thiazole in particular 2-phenyl-thiazol-4-yl; unsubstituted 2-pyridinyl; and 2-methyl-oxazole in particular 2-methyl-oxazol-4-yl.

Preferred R¹ groups is 2-isopropyl-2,4-dihydro-[1,2,4]triazol-3-one in particular N-[3-(1-isopropyl-5-oxo-4,5-dihydro-1H-[1,2,4]triazol-3-yl.

In one embodiment R¹ is not imidazol-2-yl or a bicyclic heterocycle composed of an imidazol-2-yl fused to another ring including benzimidazol-2-yl, 4,5,6,7-tetrahydroimidazo[4,5-c]pyridine, 1,4,6,7-tetrahydro-thiopyrano[3,4-d]imidazol-2-yl, 4-oxo-4,5-dihydro-1H-imidazo[4,5-c]pyridin-2-yl, 4-oxo-4,5-dihydro-1H-imidazo[4,5-d]pyridazin-2-yl, 6-oxo-5,6-dihydro-1H-imidazo[4,5-c]pyridin-2-yl and aza-benzimidazol-2-yls such as 3-(1H-imidazo[4,5-d]pyridazin-2-yl, imidazo[4,5-c]pyridin-2-yl, purin-8-yl, and 4-oxo-3,4,5,6,7,8-hexahydro-1,3,5-triaza-azulen-2-yl or a tricyclic heterocycle composed of an imidazol-2-yl fused to another ring including 1,5,6,7-tetrahydroindeno[5,6-d]imidazole.

R²

In one embodiment R² is not a C₁₋₄ hydrocarbyl group substituted by halogen (e.g. fluorine), in particular methyl substituted by halogen i.e CF₃

In one embodiment when R² is a C₁₋₄ hydrocarbyl group substituted by halogen (e.g. fluorine), in particular methyl substituted by halogen i.e CF₃, R¹ is a heteroaryl.

In a further embodiment R² is hydrogen; halogen; C₁₋₄ alkoxy (e.g. methoxy) or an unsubstituted C₁₋₄ hydrocarbyl group.

In one embodiment, R² is hydrogen or unsubstituted C₁₋₄ hydrocarbyl groups such as methyl.

Preferably R² is hydrogen.

R³

R³ is selected from optionally substituted carbocyclic and heterocyclic groups having from 3 to 12 ring members or an optionally substituted C₁₋₈ hydrocarbyl group.

In one embodiment R³ is selected from optionally substituted carbocyclic and heterocyclic groups having from 3 to 12 ring members. For example, R³ can be a monocyclic or bicyclic group having from 3 to 10 ring members.

In one embodiment R³ is a monocyclic group with 3 to 7 ring members, more usually 3 to 6 ring members, for example, 3, 4, 5 or 6 in particular 5 or 6 ring members.

In one embodiment R³ is an aromatic group, in particular an aromatic monocyclic with 3 to 7 ring members, more usually 3 to 6 ring members, for example, 3, 4, 5 or 6.

In another embodiment the monocyclic group R³ is an aryl group having 6 ring members.

Preferred aryl groups R³ are unsubstituted or substituted phenyl groups.

In one embodiment R³ is mono-, di- or tri-substituted, preferably di-substituted. In one embodiment R³ is 2,3 disubstituted, 2,5 disubstituted, 2,6 disubstituted, 3,5 disubstituted or 2,4,6, trisubstituted phenyl. In one embodiment R³ is disubstituted in particular 2,5 disubstituted, 2,6 disubstituted, or 3,5 disubstituted.

In one embodiment the substituents on the phenyl group are selected from halogen, and a group R^{a}-R^{b} wherein R^{a} is a bond, O, S, SO, SO₂, SO₂NR^{c} or NR^{c}SO₂; and R^{b} is carbocyclic and heterocyclic groups having from 3 to 12 ring members, and a C₁₋₈ hydrocarbyl group.

In another embodiment the substituents on the phenyl group are selected from halogen, and a group R^{a}-R^{b} wherein R^{a} is a bond, O, SO_{2;} and R^{b} is heterocyclic groups having from 5 to 7 ring members, and a C₁₋₄ hydrocarbyl group.

In another embodiment the substituents on the phenyl group are selected from halogen, and a group R^{a}-R^{b} wherein R^{a} is a bond, O, SO₂; and R^{b} is non-aromatic heterocyclic groups having 6 ring members e.g. N-methyl piperazine, and a C₁₋₄ hydrocarbyl group e.g. methyl.

Preferred phenyl substituents are fluorine, chlorine, methanesulfonyl, N-methyl piperazine and methoxy.

In one embodiment, a phenyl group R³ may be disubstituted at positions 2- and 6- with substituents selected from fluorine, chlorine and R^{a}-R^{b}, where R^{a} is O and R^{b} is C₁₋₄ alkyl, with fluorine being a particular substituent.

Preferably R³ is selected from 2,6-difluorophenyl, 2-fluoro-6-methoxyphenyl, 2-chloro-6-fluorophenyl, 2,6-dichlorophenyl, 2-methoxy-5-chlorophenyl, 2-methoxy-5-methanesulfonylphenyl, 2,4,6-trifluorophenyl, 2,6-difluoro-4-methoxyphenyl, 3-fluoro-5-(4-methyl-piperazin-1-yl)-phenyl and 2,3-dihydro-benzo[1,4]dioxine.

R³ is further selected from 2,6-difluorophenyl, 2-fluoro-6-methoxyphenyl, 2-methoxy-5-chlorophenyl, 2-methoxy-5-methanesulfonylphenyl, 2,6-dichlorophenyl, and 3-fluoro-5-(4-methyl-piperazin-1-yl)-phenyl.

Particularly preferred groups R³ are 2,6-difluorophenyl or is 2,6-dichlorophenyl.

In another embodiment the monocyclic group R³ is a non-aromatic carbocyclic group, having from 3 to 7 ring members, more usually 3 to 6 ring members, for example, 3, or 4, or 5, or 6 ring members. The non-aromatic carbocyclic group may be saturated or partially unsaturated but preferably it is saturated, i.e. R³ is a cycloalkyl group e.g. unsubstituted cyclopropyl or cyclohexyl.

In one particular embodiment, R³ is cyclopropyl.

In one embodiment the monocyclic group R³ is an optionally substituted or unsubstituted heteroaryl group having 5 or 6 ring members.

In one subgroup of compounds, the group R³ is a five membered heteroaryl group containing 1 or 2 ring heteroatoms selected from O, N and S. Particular heteroaryl groups include furan, thiophene, pyrrole, oxazole, isoxazole and thiazole groups. The heteroaryl groups may be unsubstituted or substituted by one or more substituent groups as hereinbefore defined.

In one sub-group of compounds, R³ is an optionally substituted or unsubstituted heteroaryl group which has 5 ring members, in particular the monocyclic group R³ is furanyl (e.g. 2-furanyl and 3-furanyl) in particular substituted 2-furanyl. In a further embodiment R³ is 5-methyl-4-morpholin-4-ylmethyl-furan-2-yl or 5-piperidin-1-ylmethyl-furan-2-yl.

In another sub-group of compounds, the heteroaryl group has 6 ring members, particular R³ is pyrazine.

The monocyclic heteroaryl groups R³ typically have up to 4 ring heteroatoms selected from N, O and S, and more typically up to 3 ring heteroatoms, for example 1, or 2, or 3 ring heteroatoms.

The aryl and heteroaryl R³ groups are therefore preferably selected from optionally substituted phenyl; optionally substituted furanyl (e.g. 2-furanyl and 3-furanyl) in particular substituted 2-furanyl, and optionally substituted pyrazine.

In one embodiment R³ is a non-aromatic monocyclic heterocyclic group having from 4 to 7 ring members and more preferably 5 or 6 ring members. The non-aromatic monocyclic heterocyclic groups will typically contain up to 3 ring heteroatoms, more usually 1 or 2 ring heteroatoms, selected from N, S and O. The heterocyclic group may be saturated or partially unsaturated, but preferably it is saturated.

Examples of non-aromatic groups R³ include monocyclic cycloalkyl and azacycloalkyl groups such as cyclohexyl, cyclopentyl and piperidinyl, particularly cyclohexyl and 4-piperidinyl groups. Other examples of non-aromatic groups R³ include monocyclic oxacycloalkyl groups such as tetrahydrofuranyl and aza-oxa cycloalkyl groups such as morpholino (e.g. 2-morpholino and 4-morpholino).

In one embodiment R³ is a non-aromatic monocyclic oxacycloalkyl heterocyclic group having 5 ring members in particular tetrahydrofuran.

In another embodiment R³ is a non-aromatic group selected from monocyclic cycloalkyl groups such as cyclohexyl, cyclopropyl and oxacycloalkyl group such as tetrahydrofuran.

Where R³ is a bicyclic group, typically it has 8 to 10 ring members, for example 8, or 9, or 10 ring members. The bicyclic group can be an aryl or heteroaryl group and examples of such groups include groups comprising a 5-membered ring fused to another 5-membered ring; a 5-membered ring fused to a 6-membered ring; and a 6-membered ring fused to another 6-membered ring.

A bicyclic aryl or heteroaryl group can comprise two aromatic or unsaturated rings, or one aromatic and one non-aromatic (e.g. partially saturated) ring.

Bicyclic heteroaryl groups typically contain up to 4 heteroatom ring members selected from N, S and O. Thus, for example, they may contain 1, or 2, or 3, or 4 heteroatom ring members.

In the monocyclic and bicyclic heterocyclic groups R³, examples of combinations of heteroatom ring members include N; NN; NNN; NNNN; NO; NNO; NS, NNS, O, S, OO and SS. Preferred combinations are N; NN; and O.

The moiety R³ may be substituted by more than one substituent. Thus, for example, there may be 1 or 2 or 3 or 4 substituents, more typically 1, 2 or 3 substituents. In one embodiment, where R³ is a six membered ring (e.g. a carbocyclic ring such as a phenyl ring), there may be a single substituent which may be located at any one of the 2-, 3- and 4-, 5- or 6-positions on the ring. In another embodiment, there may be two or three substituents and these may be located at the 2-, 3-, 4-, 5- or 6-positions around the ring. By way of example, a phenyl group R³ may be 2-monosubstituted, 3-monosubstituted, 2,6-disubstituted, 2,3-disubstituted, 2,4-disubstituted 2,5-disubstituted, 2,3,6-trisubstituted or 2,4,6-trisubstituted.

The group R³ can be an unsubstituted or substituted carbocyclic or heterocyclic group in which one or more substituents can be selected from the group R¹⁰ as hereinbefore defined. In one embodiment, the substituents on R³ may be selected from the group R^{30a} consisting of halogen, hydroxy, trifluoromethyl, cyano, nitro, carboxy, heterocyclic groups having 5 or 6 ring members and up to 2 heteroatoms selected from O, N and S, a group R^{a}-R^{b} wherein R^{a} is a bond, O, CO, X³C(X⁴), C(X⁴)X³, X³C(X⁴)X³, S, SO, or SO₂, and R^{b} is selected from hydrogen, heterocyclic groups having 5 or 6 ring members and up to 2 heteroatoms selected from O, N and S, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In a further embodiment, the substituents on R³ may be selected from the group R^{30b} consisting of halogen, hydroxy, trifluoromethyl, cyano, nitro, carboxy, a group R^{a}-R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In another embodiment, the substituents on R³ may be selected from halogen, hydroxy, trifluoromethyl, a group R^{a}-R^{b} wherein R^{a} is a bond or O, and R^{b} is selected from hydrogen and a C₁₋₄ hydrocarbyl group optionally substituted by one or more substituents selected from hydroxyl and halogen.

One sub-set of substituents that may be present on a group R³ (e.g. an aryl or heteroaryl group R³) includes fluorine, chlorine, methoxy, ethoxy, methyl, ethyl, isopropyl, tert-butyl, trifluoromethyl, difluoromethoxy, trifluoromethoxy, amino, N-methylpiperazino, piperazine, piperidino, piperidinomethyl, pyrrolidino, pyrrolidinylmethyl, morpholino, and morpholinomethyl.

In one embodiment the group R³ is substituted by one or more substitutents i.e. 1, 2, or 3 substitutents preferably 1 or 2 substitutents selected from halogen, and a group R^{a}-R^{b} wherein R^{a} is a bond, O, S, SO, SO₂, or SO₂NR^{c}; and R^{b} is carbocyclic and heterocyclic groups having from 3 to 12 ring members, and a C₁₋₈ hydrocarbyl group optionally substituted by one or more substituents selected from hydroxy, oxo, halogen, amino, mono- or di-C₁₋₄ hydrocarbylamino, carbocyclic and heterocyclic groups having from 3 to 12 ring members.

In another embodiment the R³ substituents are selected from halogen, and a group R^{a}-R^{b} wherein R^{a} is a bond, O, SO₂; and R^{b} is heterocyclic groups having from 5 to 7 ring members, and a C₁₋₄ hydrocarbyl group optionally substituted by one or more substituents selected from hydroxy, halogen, and monocyclic heterocyclic groups having from 3 to 7 ring members.

In another embodiment the R³ substituents are selected from halogen, and a group R^{a}-R^{b} wherein R^{a} is a bond, O, SO₂; and R^{b} is heterocyclic groups having from 5 to 7 ring members, and a C₁₋₄ hydrocarbyl group optionally substituted by a monocyclic non-aromatic heterocyclic groups having from 3 to 7 ring members. In another embodiment the R³ substituents are selected from halogen, and a group R^{a}-R^{b} wherein R^{a} is a bond, O, SO₂; and R^{b} is heterocyclic groups having from 5 to 7 ring members, and a C₁₋₄ hydrocarbyl group optionally substituted a monocyclic non-aromatic heterocyclic groups having from 5 to 6 ring members.

In another embodiment the R³ substituents are selected from halogen, and a group R^{a}-R^{b} wherein R^{a} is a bond, O, SO₂; and R^{b} is non-aromatic heterocyclic groups having 6 ring members e.g. N-methyl piperazine, and a C₁₋₄ hydrocarbyl group e.g. methyl optionally substituted with morpholine or piperidine.

Preferred R³ substituents are fluorine, chlorine, methanesulfonyl, N-methyl piperazine, 4-morpholin-4-ylmethyl, piperidin-1-ylmethyl, methyl and methoxy.

Where the carbocyclic and heterocyclic groups have a pair of substituents on adjacent ring atoms, the two substituents may be linked so as to form a cyclic group. Thus, two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S. In particular the two adjacent groups R¹⁰, together with the carbon atoms or heteroatoms to which they are attached, may form a 6-membered non-aromatic heterocyclic ring, containing up to 3, in particular 2, heteroatom ring members selected from N, O and S. More particularly the two adjacent groups R¹⁰ may form a 6-membered non-aromatic heterocyclic ring, containing 2 heteroatom ring members selected from N, or O, such as dioxan e.g. [1,4 dioxan]. In one embodiment R¹ is a carbocyclic group e.g. phenyl having a pair of substituents on adjacent ring atoms linked so as to form a cyclic group e.g. to form 2,3-dihydro-benzo[1,4]dioxine.

In one embodiment R³ is an optionally substituted C₁₋₈ hydrocarbyl group.

In one embodiment R³ the substituted and unsubstituted C₁₋₈ hydrocarbyl groups is a C₁₋₈ alkyl group in particular a C₁₋₄ alkyl group such as methyl, ethyl, iso-propyl, and tertiary butyl. Preferably the C₁₋₈ hydrocarbyl group is a substituted C₁₋₄ alkyl group in particular a substituted C₁₋₂ alkyl.

Preferred optional substitutents are carbocyclic or heterocyclic groups in particular a monocyclic carbocyclic group. Preferred carbocycles are C₆₋₁₀ aryl groups such as phenyl.

Therefore a preferred substituted C₁₋₈ hydrocarbyl group is an unsubstituted or substituted aralkyl group such as benzyl.

In a further embodiment, R³ is substituted or unsubstituted C₆₋₁₀ carbocycle-C₁₋₂ alkyl in particular a C₆ carbocycle-C₁₋₂ alkyl groups such as the aralkyl group phenyl-C₁₋₂ alkyl e.g. unsubstituted or substituted benzyl or phenethyl.

A particularly preferred substituted C₁₋₈ hydrocarbyl group is an aralkyl or C₆ carbocycle-C₁₋₂ alkyl group such as benzyl which may be unsubstituted or substituted with 1 or 2 unsubstituted substituents selected from fluorine, chlorine and methoxy. In one embodiment R³ is 2,6-difluorobenzyl.

In one embodiment R³ the substituted and unsubstituted C₁₋₈ hydrocarbyl groups is a C₁₋₈ alkyl group in particular a C₁₋₄ alkyl group such as methyl, ethyl, iso-propyl, and tertiary butyl. Preferably the C₁₋₈ hydrocarbyl group is a unsubstituted C₁₋₄ alkyl group in particular an unsubstituted C₁₋₂ alkyl such as methyl.

In another embodiment R³ is an unsubstituted C₁-C₄ alkyl or a C₆ carbocycle-C₁₋₂ alkyl group in which the alkyl moiety is unsubstituted and the carbocycle is unsubstituted or substituted with 1 or 2 unsubstituted substituents selected from halogen e.g. fluorine, chlorine and C₁₋₄ alkoxy e.g. methoxy.

In one embodiment R³ is an optionally substituted phenyl; optionally substituted furanyl (e.g. 2-furanyl and 3-furanyl) in particular substituted 2-furanyl, optionally substituted tetrahydrofuran, optionally substituted pyrazine, unsubstituted or substituted benzyl, optionally substituted C₁₋₄ alkyl and optionally substituted C₃₋₆ cycloalkyl group.

In one embodiment R³ substitutents are halogen such as fluorine, chlorine, methanesulfonyl, 5-7 membered, C₁₋₄ alkoxy such as methoxy, and 5-7 membered unsaturated heterocycle such as N-methyl piperazine, morpholine or piperidine, and optionally substituted C₃₋₆ carbocycle where the substituents are selected from halogen such as fluorine, chlorine, methanesulfonyl, 5-7 membered, C₁₋₄ alkoxy such as methoxy, and 5-7 membered unsaturated heterocycle such as N-methyl piperazine, morpholine or piperidine.

Preferred R³ substitutents are halogen such as fluorine, chlorine, methanesulfonyl, 5-7 membered, C₁₋₄ alkoxy such as methoxy, and 5-7 membered unsaturated heterocycle such as N-methyl piperazine, morpholine or piperidine, and optionally substituted C₃₋₆ carbocycle where the substituents are selected from halogen such as fluorine, chlorine, methanesulfonyl, 5-7 membered, C₁₋₄ alkoxy such as methoxy, and 5-7 membered unsaturated heterocycle such as N-methyl piperazine, morpholine or piperidine.

In one embodiment R³ is a disubstituted phenyl where the substituents are substituents are fluorine, chlorine, methanesulfonyl, 5-7 membered unsaturated heterocycle such as N-methyl piperazine and methoxy; mono- or disubstituted 2-furanyl where the substituents are unsubsituted or substituted methyl where the substituent is 5-7 membered unsaturated heterocycle such as morpholine or piperidine; unsubstituted tetrahydrofuran; unsubstituted pyrazine; disubstituted benzyl where the substituents are substituents are fluorine, chlorine, methanesulfonyl, 5-7 membered unsaturated heterocycle such as N-methyl piperazine and methoxy; unsubstituted methyl; unsubstituted cyclopropyl and unsubstituted cyclohexyl.

In a further embodiment R³ is selected from 2,6-difluorophenyl, 2,6-difluorobenyl, 2-fluoro-6-methoxyphenyl, 2-methoxy-5-chlorophenyl, 2-methoxy-5-methanesulfonylphenyl, 2,6-dichlorophenyl, 3-fluoro-5-(4-methyl-piperazin-1-yl)-phenyl, 5-methyl-4-morpholin-4-ylmethyl-furan-2-yl, 5-piperidin-1-ylmethyl-furan-2-yl, unsubstituted methyl, unsubstituted tetrahydrofuran-2-yl; unsubstituted pyrazine such as 1,4-pyrazin-2-yl, unsubstituted cyclopropyl and unsubstituted cyclohexyl.

In one embodiment R³ is an optionally substituted phenyl; optionally substituted furanyl (e.g. 2-furanyl and 3-furanyl) in particular substituted 2-furanyl, optionally substituted tetrahydrofuran, and optionally substituted C₃₋₆ cycloalkyl group.

In one embodiment R³ is a disubstituted phenyl where the substituents are substituents are fluorine, chlorine, methanesulfonyl, and methoxy; mono- or disubstituted 2-furanyl where the substituents are unsubsituted or substituted methyl where the substituent is 5-7 membered unsaturated heterocycle such as morpholine or piperidine; unsubstituted tetrahydrofuran; and unsubstituted cyclopropyl.

In a further embodiment R³ is selected from 2,6-difluorophenyl, 2-methoxy-5-chlorophenyl, 2-fluoro-6-methoxyphenyl, 2-methoxy-5-methanesulfonylphenyl, 2,6-dichlorophenyl, 5-piperidin-1-ylmethyl-furan-2-yl, 5-methyl-4-morpholin-4-ylmethyl-furan-2-yl, unsubstituted tetrahydrofuran-2-yl and unsubstituted cyclopropyl.

In one embodiment R³ is an optionally substituted phenyl; and optionally substituted 2-furanyl.

In one embodiment R³ is a disubstituted phenyl where the substituents are substituents are fluorine, chlorine, methanesulfonyl, and methoxy; mono-substituted 2-furanyl where the substituents is methyl substituted with 5-7 membered unsaturated heterocycle such as morpholine or piperidine.

In a further embodiment R³ is selected from 2,6-difluorophenyl, 2-methoxy-5-methanesulfonylphenyl, 2,6-dichlorophenyl, and 5-piperidin-1-ylmethyl-furan-2-yl.

In one embodiment, the compounds of the invention are represented by the formula (II):

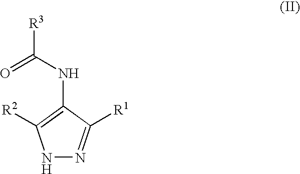

wherein R¹, R² and R³ are each independently selected from R¹, R², and R³ as defined herein in respect of formula (I) and subgroups, examples and preferences thereof.

Another embodiment of the invention can be represented by the formula (III):

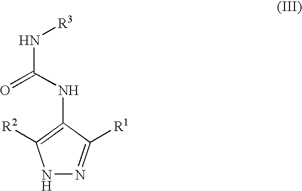

wherein R¹, R² and R³ are each independently selected from R¹, R², and R³ as defined herein in respect of formula (I) and subgroups, examples and preferences thereof.

Within formula (II) and formula (III), it is preferred that R² is hydrogen.

Within the group of compounds defined by the formula (III), R³ is preferably C₃₋₆ cycloalkyl or aralkyl.

More particularly, R³ is selected from cyclopropyl or 2,6-difluorobenzyl.

In one preferred embodiment of formula (III), R³ is 2,6-difluorobenzyl.

In another preferred embodiment of formula (III), R³ is cyclopropyl.

Within the group of compounds defined by the formula (III), R¹ is preferably 2-pyridinyl.

For the avoidance of doubt, it is to be understood that each general and specific preference, embodiment and example of the groups R¹ may be combined with each general and specific preference, embodiment and example of the groups R² and/or R³ and/or R¹⁰ and/or Y and/or A and/or B and/or R^{g} and/or sub-groups thereof as defined herein and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below.

Salts, Solvates, Tautomers, Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (II) and (III) and sub-groups thereof unless the context indicates otherwise.

The salts of the present invention can be synthesized from the parent compound that contains a basic or acidic moiety by conventional chemical methods such as according to methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, undecylenic and valeric acids, as well as acylated amino acids and cation exchange resins.

The acid addition salts salts may also be selected from aspartic (e.g. D-aspartic), carbonic, dodecanoic, isobutyric, laurylsulphonic, mucic, naphthalenesulphonic (e.g. naphthalene-2-sulphonic), toluenesulphonic (e.g. p-toluenesulphonic), and xinafoic acids.

One particular group of salts consists of salts formed from hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

One preferred group of salts consists of salts formed from hydrochloric, acetic, adipic, L-aspartic and DL-lactic acids.

Particularly Preferred Salts are Hydrochloride Salts

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, in compounds of the formula (I) the pyrazole group may take either of the following two tautomeric forms A and B. For simplicity, the general formula (I) illustrates form A but the formula is to be taken as embracing both tautomeric forms.

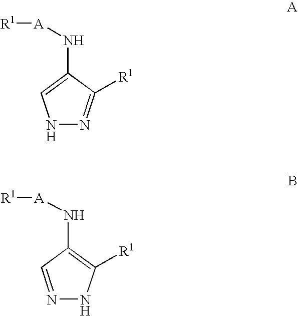

Other examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

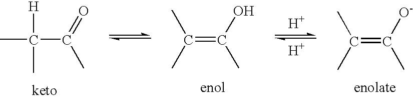

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Calm, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl

(e.g., -Me, -Et, -nPr, -iPr, -nBu, -sBu, -iBu, -tBu);

C₁₋₇aminoalkyl

(e.g., aminoethyl; 2-(N,N-diethylamino)ethyl; 2-(4-morpholino)ethyl); and

acyloxy-C₁₋₇alkyl

(e.g., acyloxymethyl;

acyloxyethyl;

pivaloyloxymethyl;

acetoxymethyl;

1-acetoxyethyl;

1-(1-methoxy-1-methyl)ethyl-carbonxyloxyethyl;

1-(benzoyloxy)ethyl; isopropoxy-carbonyloxymethyl;

1-isopropoxy-carbonyloxyethyl; cyclohexyl-carbonyloxymethyl;

1-cyclohexyl-carbonyloxyethyl;

cyclohexyloxy-carbonyloxymethyl; 1-cyclohexyloxy-carbonyloxyethyl;

(4-tetrahydropyranyloxy) carbonyloxymethyl;

1-(4-tetrahydropyranyloxy)carbonyloxyethyl;

(4-tetrahydropyranyl)carbonyloxymethyl; and

1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Biological Activity

The compounds of the formulae (I) and sub-groups thereof are inhibitors of cyclin dependent kinases. For example, compounds of the invention have activity against CDK1, CDK2, CDK3, CDK4, CDK5, CDK6 and CDK7 kinases, and in particular cyclin dependent kinases selected from CDK1, CDK2, CDK3, CDK4, CDK5 and CDK6.

Preferred compounds are compounds that inhibit one or more CDK kinases selected from CDK1, CDK2, CDK4 and CDK5, for example CDK1 and/or CDK2.

In addition, CDK4, CDK8 and/or CDK9 may be of interest.

Compounds of the invention also have activity against glycogen synthase kinase-3 (GSK-3).

Compounds of the invention also have activity against Aurora kinases.

As a consequence of their activity in modulating or inhibiting CDK and Aurora kinases and glycogen synthase kinase, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds of the invention will be useful in treating conditions such as viral infections, type II or non-insulin dependent diabetes mellitus, autoimmune diseases, head trauma, stroke, epilepsy, neurodegenerative diseases such as Alzheimer's, motor neurone disease, progressive supranuclear palsy, corticobasal degeneration and Pick's disease for example autoimmune diseases and neurodegenerative diseases.

One sub-group of disease states and conditions where it is envisaged that the compounds of the invention will be useful consists of viral infections, autoimmune diseases and neurodegenerative diseases.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors. RB−ve tumours may also be sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xeroderma pigmentoum; keratoctanthoma; thyroid follicular cancer; or Kaposi's sarcoma.

The cancers may be cancers which are sensitive to inhibition of any one or more cyclin dependent kinases selected from CDK1, CDK2, CDK3, CDK4, CDK5 and CDK6, for example, one or more CDK kinases selected from CDK1, CDK2, CDK4 and CDK5, e.g. CDK1 and/or CDK2.

Whether or not a particular cancer is one which is sensitive to inhibition by a cyclin dependent kinase may be determined by means of a cell growth assay as set out in Example 250 below or by a method as set out in the section headed “Methods of Diagnosis”.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-senstive rhinosinusitis, cystic fibrosis, multiple sclerosis, kidney diseases and cancer pain.

It has also been discovered that some cyclin-dependent kinase inhibitors can be used in combination with other anticancer agents. For example, the cyclin-dependent kinase inhibitor flavopiridol has been used with other anticancer agents in combination therapy.

Thus, in the pharmaceutical compositions, uses or methods of this invention for treating a disease or condition comprising abnormal cell growth, the disease or condition comprising abnormal cell growth in one embodiment is a cancer.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes breast cancer, ovarian cancer, colon cancer, prostate cancer, oesophageal cancer, squamous cancer and non-small cell lung carcinomas.

In the case of compounds having activity against Aurora kinase, particular examples of cancers where it is envisaged that the Aurora kinase inhibiting compounds of the invention will be useful include:

human breast cancers (e.g. primary breast tumours, node-negative breast cancer, invasive duct adenocarcinomas of the breast, non-endometrioid breast cancers);

ovarian cancers (e.g. primary ovarian tumours);

pancreatic cancers;

human bladder cancers;

colorectal cancers (e.g. primary colorectal cancers);

gastric tumours;

renal cancers;

cervical cancers:

neuroblastomas;

melanomas;

lymphomas;

prostate cancers;

leukemia;

non-endometrioid endometrial carcinomas;

gliomas;

non-Hodgkin's lymphoma;

Cancers which may be particularly amenable to Aurora inhibitors include breast, bladder, colorectal, pancreatic, ovarian, non-Hodgkin's lymphoma, gliomas and nonendometrioid endometrial carcinomas. One particular cancer is breast cancer e.g. primary breast tumours, node-negative breast cancer, invasive duct adenocarcinomas of the breast, non-endometrioid breast cancers.

A particular sub-set of cancers which may be particularly amenable to Aurora inhibitors consist of breast, ovarian, colon, liver, gastric and prostate cancers.

The activity of the compounds of the invention as inhibitors of cyclin dependent kinases, Aurora kinases and glycogen synthase kinase-3 can be measured using the assays set forth in the examples below and the level of activity exhibited by a given compound can be defined in terms of the IC₅₀ value. Preferred compounds of the present invention are compounds having an IC₅₀ value of less than 1 micromole, more preferably less than 0.1 micromole.

Another subset of cancers that Aurora inhibitors may be particularly amenable to treat are hematological cancers, in particular leukemia. Therefore, in a further embodiment the compounds of formula (I) are used to treat hematological cancers, in particular leukemia. Particular leukemias are selected from Acute Myelogenous Leukemia (AML), chronic myelogenous leukaemia (CML), B-cell lymphoma (Mantle cell), and Acute Lymphoblastic Leukemia (ALL). In one embodiment the leukemias are selected from relapsed or refractory acute myelogenous leukemia, myelodysplastic syndrome, acute lymphocytic leukemia and chronic myelogenous leukemia.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma).

One particular cancer is chronic lymphocytic leukaemia.

Another particular cancer is mantle cell lymphoma.

Another particular cancer is diffuse large B cell lymphoma.

It is further envisaged that the compounds of the invention, and in particular those compounds having aurora kinase inhibitory activity, will be particularly useful in the treatment or prevention of cancers of a type associated with or characterised by the presence of elevated levels of aurora kinases, for example the cancers referred to in this context in the introductory section of this application.

Methods for the Preparation of Compounds of the Formula (I)

Compounds of the formula (I) can be prepared in accordance with synthetic methods well known to the skilled person.

Unless stated otherwise Y, A, B, R¹, R², R³ and R¹⁰ are as herein defined.

The required starting materials for Schemes 1-8 can be obtained commercially or can be prepared from appropriately substituted precursor compounds using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995.

Compounds of the formula (I) wherein A is —Y—(B)ₙ—, n is 1 and B is C═O can be prepared as illustrated in Scheme 1 below.

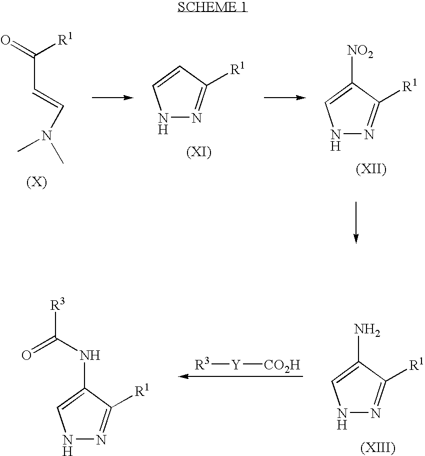

Heating the appropriate 3-dimethylamino-propenone with the appropriate hydrazine hydrate gives a pyrazole of formula (XI). The required α,β-unsaturated ketone (X) can be obtained commercially or can be generated from the appropriate ketone by reaction with dimethylformamide-dimethylacetal at elevated temperature (Jachak et al, Montash. Chem., 1993, 124(2), 199-207). The pyrazole (XI) can then be nitrated by reaction in concentrated sulfuric acid and fuming nitric acid to give the nitropyrazole (XII). Amines of the formula (XIII) can be prepared by reduction of the corresponding nitro-compound (XII) under standard conditions. The reduction may be effected, for example by catalytic hydrogenation in the presence of a catalyst such as palladium on carbon in a polar solvent such as ethanol or dimethylformamide at room temperature. As an alternative, reduction may be effected using a reducing agent such as tin (II) chloride in ethanol, typically with heating, for example to the reflux temperature of the solvent. Some compounds of formula (XI) e.g. 2-(1H-pyrazole-3-yl)pyridine are commercially available and thus can be nitrated and reduced without the need to perform the initial synthetic step.

As shown in Scheme 1, an amine of the formula (XIII) can be reacted with a carboxylic acid, or reactive derivative thereof, of the formula R³—Y—CO₂H under standard amide formation conditions. Carboxylic acids of the formula R³—Y—CO₂H can be obtained commercially or can be synthesised according to methods well known to the skilled person, see for example Advanced Organic Chemistry by Jerry March, 4^{th} Edition, John Wiley & Sons, 1992, and Organic Syntheses, Volumes 1-8, John Wiley, edited by Jeremiah P. Freeman (ISBN: 0-471-31192-8), 1995. Thus, for example, the coupling reaction between the carboxylic acid and the amine (XIII) can be carried out in the presence of a reagent of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (EDC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) (L. A. Carpino, J. Amer. Chem. Soc., 1993, 115, 4397) and phosphonium-based coupling agents such as 1-benzo-triazolyloxytris(pyrrolidino)phosphonium hexafluorophosphate (PyBOP) (Castro et al, Tetrahedron Letters, 1990, 31, 205). Carbodiimide-based couling agents are advantageously used in combination with 1-hydroxyazabenzotriazole (HOAt) or 1-hydroxybenzotriazole (HOBO (Konig et al, Chem. Ber., 103, 708, 2024-2034). Preferred coupling reagents include EDC and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine or triethylamine.

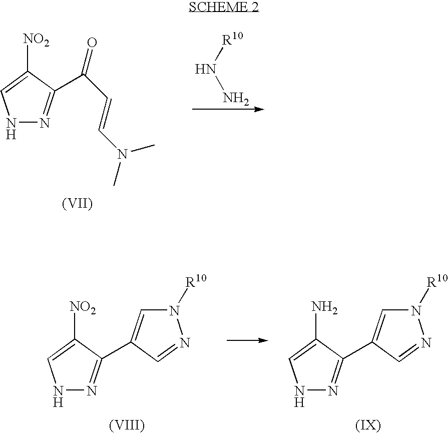

Alternatively, the same cyclisation reaction can be used to synthesise the R¹ heterocyclic group where R¹ is pyrazole as outlined in Scheme 2 above. 3-(Dimethylamino)-2-{4-nitro-1H-pyrazol-3-yl}acrylaldehyde (VII) and the appropriately substituted hydrazine are heated to cyclise to form the substituted di-pyrazole (VIII). For example, (3-morpholin-4-yl-propyl)-hydrazine could be used to generate compounds where R¹⁰ is 3-morpholin-4-yl-propyl. The nitro group of compound (VIII) can then be reduced to the amine (IX) and coupled with the carboxylic acid or acid chloride to give the compound of formula I as described above.

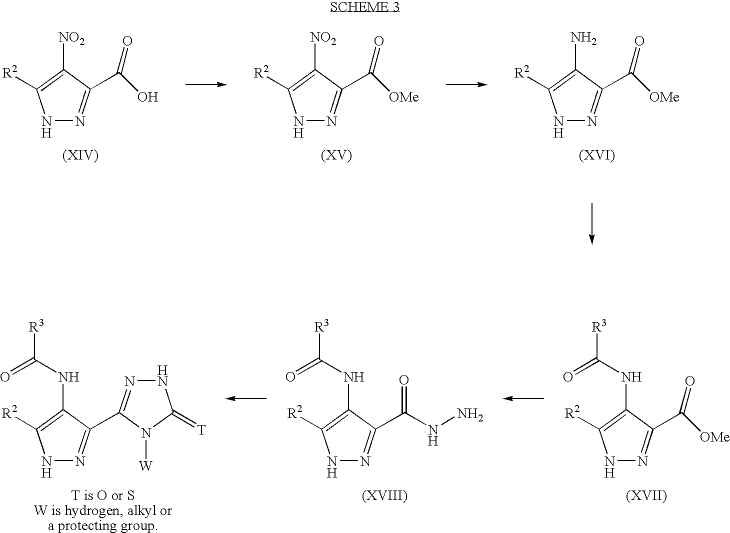

The starting material for the synthetic route shown in Scheme 3 is the 4-nitro-pyrazole-3-carboxylic acid (XIV) which can either be obtained commercially or can be prepared by nitration of the corresponding 4-unsubstituted pyrazole carboxy compound. As shown in Scheme 3 a substituted or unsubstituted 4-nitro-3-pyrazole carboxylic acid (XIV) can be esterified by reaction with thionyl chloride to give the acid chloride intermediate followed by reaction with the required alcohol e.g. methanol to form the methyl ester (XV). The methyl ester is shown in Scheme 3 but it could equally be ethyl ester. Alternatively, the esterification can be carried out by reacting the alcohol and carboxylic acid in the presence of an acidic catalyst, one example of which is thionyl chloride. The reaction is typically carried out at room temperature using the esterifying alcohol (e.g. ethanol) as the solvent.

The nitro group can then be reduced using palladium on carbon according to standard methods to give the amine (XVI). The amine (XVI) is coupled with an appropriate carboxylic acid R³—Y—CO₂H under amide forming conditions the same as or analogous to those described above to give the amide (XVII). The carboxylic acid ester (XVII) is then reacted with hydrazine monohydrate in solvent e.g. ethanol and heated at reflux to give the hydrazinocarbonyl (XVIII). The hydrazinocarbonyl is then reacted with an isocyanate e.g. para-methoxybenzylisocyanate in solvent e.g. anhydrous THF under an inert atmosphere at ambient temperature. The mixture gives the intermediate hydrazide which can then be reacted in aqueous NaOH solution at reflux prior to quenching in saturated aqueous NH₄Cl to give, once any protecting groups present have been removed, compounds where 5-oxo-4,5-dihydro-1H-[1,2,4]triazol-3-yl is the R¹ group. 5-Thioxo-4,5-dihydro-1H-[1,2,4]triazol-3-yl can be sythesised from this cyclisation reaction by treating the hydrazinocarbonyl (XVIII) with isothiocyanate in solvent e.g. 1-butanol with the addition of DBU. 4-alkyl-5-thioxo-4,5-dihydro-1H-[1,2,4]triazol-3-yl can be sythesised by use of alkyl isothiocyanate in this reaction.

Alternatively the reactions can be carried out in a different order as shown below in Scheme 4 where the cyclisation reaction to form R¹ is performed first followed by reaction to generate the amide.

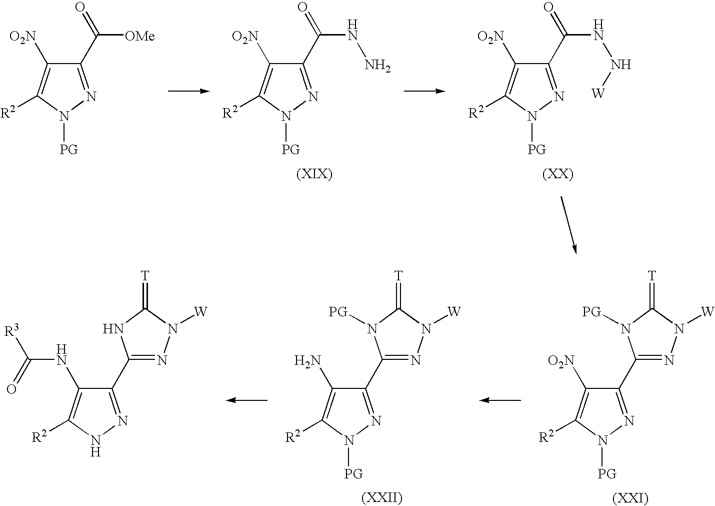

The nitrogen in the pyrazole ring of optionally substituted 4-nitro-1H-pyrazole-3-carboxylic ester (XV) can be protected with an appropriate protecting groups for example THP or PMB and then reacted with hydrazine hydrate and ethanol and stirred at room temperature to produce the hydrazinocarbonyl (XIX). Protected isocyanate or isothiocyanate e.g. paramethoxybenzyl isocyanate is added at 0° C. to the hydrazinocarbonyl (XIX) to give the [1,2,4]triazol-3-(thi)one (XXI). Where W is an alkyl group e.g. isopropyl, the hydrazinocarbonyl (XIX) can be reacted with a ketone e.g. acetone and NaCNBH₃ in solvent to give the alkyl-hydrazinocarbonyl (XX) prior to the cyclisation step or alkyl-iso(thio)cyanate can be used in the cyclisation reaction with hydrazinocarbonyl (XIX). Alternatively to produce compounds of formula I where W is alkyl following the cyclisation reaction of the hydrazinocarbonyl (XIX) with iso(thio)cyanate the compound can be alkylated by alkylation of the triazol-3-(thi)one ring with a alkylating agent e.g. methyl iodide or 4- (3-chloroethyl)-morpholine. The nitro-group of compound (XXI) can then be reduced to amine (XXII) for reaction with the corresponding carboxylic acid to give the amide as described previously. Removal of any protecting groups then generates the compound of Formula I.

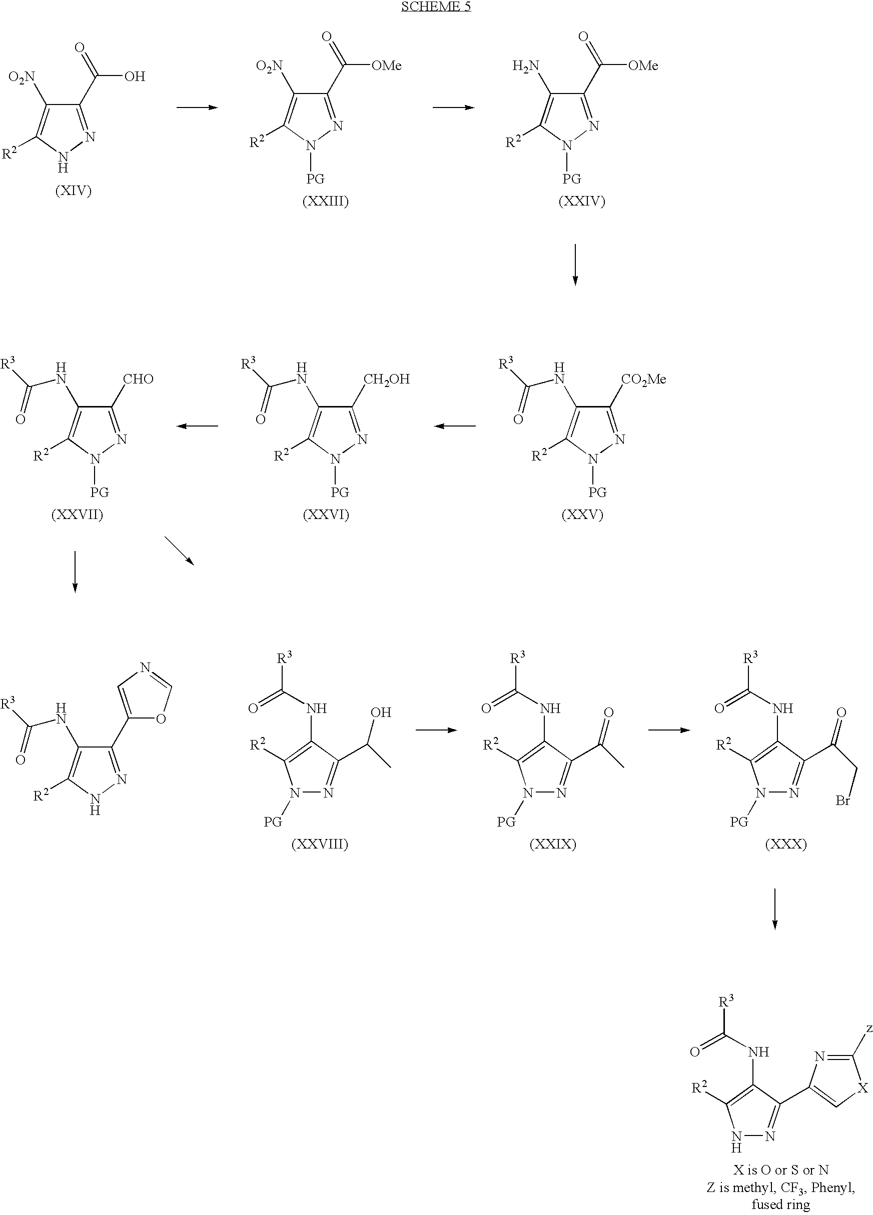

Compounds of formula I where R¹ is thiazolyl, oxazolyl or 1H-imidazol-4-yl can be synthesised as shown in Scheme 5. The nitrogen in the pyrazole ring of 4-nitro-1H-pyrazole-3-carboxylic ester (XIV) can be protected for example with a THP to give compound (XXIII) prior to the reduction of the nitro group to the amine (XXIV) as described above. The amine is then reacted with the corresponding carboxylic acid or derivative thereof as described previously to generate amide (XXV).

The pyrazole formyl of the formula (XXVII) can be prepared from the corresponding carboxylic acid, or methyl ester derivative (XXV), by reduction to the alcohol (XXVI) and then limited oxidiation to the aldehyde using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995 and Handbook of Reagents for Organic Synthesis: Oxidizing and Reducing Agents, S. D. Burke, R. L. Danheiser, John Wiley and Sons Ltd, 1999. The methyl ester compound (XXV) can be reduced to the hydroxymethyl compound (XXVI) using standard techniques for example using diisobutylaluminium hydride in a non-polar solvent such as THF at −78° C. The hydroxymethyl compound (XXVI) is then oxidised to the formyl compound (XXVII) in an aprotic solvent such as acetone using manganese oxide (MnO₂).

The formyl compound (XXVII) can then be reacted with Grignard reagent under a nitrogen atmosphere for example with methyl magnesium bromide to give the hydroxy-ethyl compound (XXVIII). This can then reacted under a nitrogen atmosphere with Dess-Martin Periodinane to give the acetyl compound (XXIX). The acetyl (XXIX) can then be brominated using standard techniques for example using bromine. The cyclisation reaction of the bromo-ketone (XXX) can be carried out in the presence of the required reagent such as an amide, amidine or ammonium acetate, to give the desired R¹ group. This cyclisation step is typically carried out by heating under reflux in the presence of acetic acid. For example, thioamide e.g. thioacetamide or thiobenzamide will give 2-substituted-thiazolyl (where X is S), whereas acetic acid, ammonium acetate and K₂CO₃ will give 2-substituted-oxazolyl (where X is O) and substituted acetamidine gives substituted 1H-imidazol-4-yl (where X is N). Bromo-acetyl (XXX) can also be reacted with the appropriate group such as an arylamine e.g. 2-amino-pyridine to produce bicyclic R¹ groups e.g. imidazo[1,2-a]pyridin-2-yl. Alternatively the formyl compound (XXVII) can be reacted in methanol with p-toluenesulphonylmethyl isocyanide and potassium carbonate to give the compound where R¹ is 3-oxazol-5-yl.

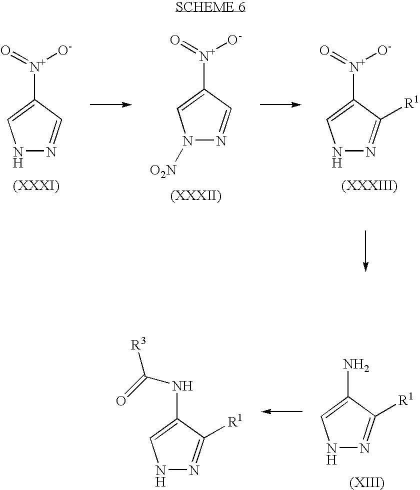

A further route to compounds of the formula (I) is shown in Scheme 6 above. The procedure illustrated in Scheme 6 is of particular utility in the preparation of compounds when R¹ is a N-linked heterocycle. 4-Nitropyrazole (XXXI) in acetic acid was nitrated with concentrated nitric acid and acetic anhydride at room to give the 1,4-dintropyrazole (XXXII). A compound then representing the desired R¹ group e.g. optionally substituted pyrazole or indazole is then added to the dinitro compound (XXXII) in solvent and heated at reflux to give 4-nitro-3-substituted-pyrazole (XXXIII). The nitro group of compound (XXXIII) can then be reduced to the amine (XIII) and reacted with the appropriate carboxylic acid or acid chloride to give the desired compound.

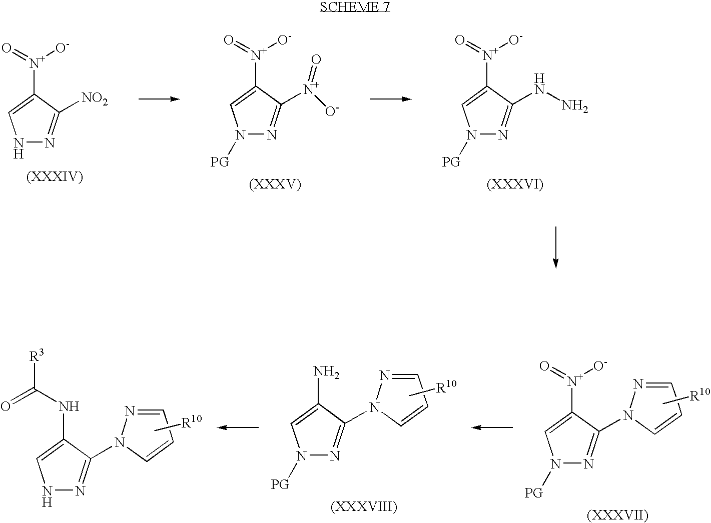

Compounds of formula I is an N-linked pyrazole can be synthesised as outlined in Scheme 7 above. 3,4-Dinitropyrazole (XXXIV) is protected e.g. by reacting it with 4-methoxybenzyl chloride and base such as potassium carbonate in acetonitrile, to give the protected derivative (XXXV). The hydrazine (XXXVI) is produced from the protected dinitro compound (XXXV) by reaction with hydrazine hydrate. The group R¹ is then produced by reaction of the hydrazine (XXXVI) in a protic solvent such as ethanol with concentrated hydrochloric acid and then the appropriate enone is added. For example 4-ethoxy-1,1,1-trifluoro-but-3-en-2-one is used where R¹ is 3- or 5-trifluoromethyl-pyrazolyl. The 4-nitro group of compound (XXXVII) is then reduced to the amine (XXXVIII) and reaction with the acid chloride or carboxylic acid will give the compound of formula I after any protecting groups present have been removed.

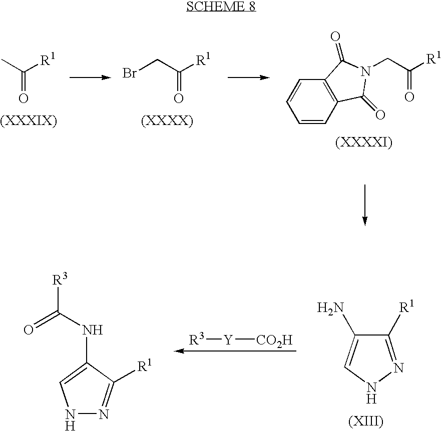

In Scheme 8, the acetyl derivative of the desired R¹ group (XXXIX) is taken, for example 2-acetylpyrazine, and brominated using standard methodologies such as by addition of glacial acetic acid and HBr/AcOH or by addition of pyridinium tribromide to give the 2-bromo-ethanone (XXXX). Alternatively the appropriate 2-bromo-ethanone (XXXX) can be made by literature methods as described in Chiarino, J. Het Chem 25(1) 337-42 1988. The 2-bromo-ethanone (XXXX) is then reacted with potassium phthalimide to give the isoindole-1,3-dione (XXXXI). The isoindole-1,3-dione is then cyclised to form the pyrazole (XIII) by heating in DMF/DMA and then treating with hydrazine hydrate. The compound of formula can then be produced by generation of the amide as described previously.

Alternatively, compounds of the formula (I) in which R¹ is a C-linked heteroaryl group can be prepared from compounds of the formula (XXXXII):

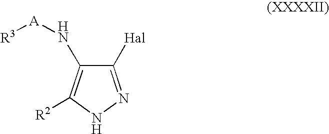

where “Hal” is a halogen such as chlorine, bromine or iodine, by means of a Suzuki coupling reaction with the appropriate heteroaryl boronate. The reaction can be carried out under typical Suzuki Coupling conditions in the presence of a palladium catalyst such as bis(tri-t-butylphosphine)palladium and a base (e.g. a carbonate such as potassium carbonate). The reaction may be carried out in an aqueous solvent system, for example aqueous ethanol, and the reaction mixture is typically subjected to heating, for example to a temperature in excess of 100° C.

Compounds of the formula (XXXXII) can be prepared from amino-pyrazole compounds by means of the Sandmeyer reaction (see Advanced Organic Chemistry, 4^{th} edition, by Jerry March, John Wiley & Sons, 1992, page 723) in which the amino group is converted to a diazonium group by reaction with nitrous acid, and the diazonium compound is then reacted with a copper (I) halide such as Cu(I)Cl or Cu(I)I.

The pyrazole starting materials for the some of the synthetic routes shown in the Schemes above, can either be obtained commercially or can be prepared by methods known to those skilled in the art. They can be obtained using known methods e.g. from ketones, such as in a process described in EP308020 (Merck), or the methods discussed by Schmidt in Helv. Chim. Acta., 1956, 39, 986-991 and Hely. Chim. Acta., 1958, 41, 306-309. Alternatively they can be obtained by conversion of a commercially available pyrazole, for example those containing halogen, nitro, ester, or amide functionalities, to pyrazoles containing the desired functionality by standard methods known to a person skilled in the art. For example, 4-nitro-pyrazole-3-carboxylic acid (XIV) can either be obtained commercially or can be prepared by nitration of the corresponding 4-unsubstituted pyrazole carboxy compound, the nitro group of 3-carboxy-4-nitropyrazole can be reduced to an amine by standard methods, and pyrazoles containing a halogen may be utilized in coupling reactions with tin or palladium chemistry.

In all of the Scheme above compounds of the formula (I) in which A is —Y—(B)ₙ— and B is NH(C═O) can be prepared using standard methods for the synthesis of ureas. For example, such compounds can be prepared by reacting an aminopyrazole compound of the formula (XIII) or (XVI) with a suitably substituted phenylisocyanate in a polar solvent such as DMF. The reaction is conveniently carried out at room temperature. Alternatively the urea can be synthesised by reaction of the amine with 1,1′-carbonyldiimidazole and the required amine e.g. cyclopropylamine.

Compounds of the formula (I) where B is O(C═O) can be made using standard methods for the synthesis of carbamates, for example by reaction of an amino pyrazole compound of the formula (XIII) or (XVI) with a chloroformate derivative of the formula R¹—O—C(O)—Cl under conditions well known to the skilled person.

Compounds of the formula (I) in which A is a bond, can be prepared from the 4-amino compounds using the Schemes described previously using standard methods for the synthesis of secondary and tertiary amines. For example, such compounds can be prepared by reacting an 4-aminopyrazole compound of the formula (XIII) or (XVI)) with a suitably substituted alkylating agent such as a compound of the formula R³-L in a nucleophilic displacement reaction where L is a leaving group such as a halogen in a polar solvent such as DMF. The reaction is conveniently carried out at room temperature. Alternatively compounds in which A is a bond can be prepared by reductive amination with an appropriately substituted aldehyde or ketone in the presence of a variety of reducing agents (see Advanced Organic Chemistry by Jerry March, 4^{th} Edition, John Wiley & Sons, 1992, pp 898-900. For example, reductive amination can be carried out in the presence of sodium triacetoxyborohydride in the presence of an aprotic solvent such as dichloromethane at or near ambient temperatures.

Once formed, one compound of the formula (I) may be transformed into another compound of the formula (I) using standard chemistry procedures well known in the art. For examples of functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995. For example, they can be prepared by converting one compound of the formula (I) into another compound of the formula (I) by reacting with alkylating agents, sulphonyl chlorides or acyl chlorides using methods known to a person skilled in the art.

In particular these methods can be used to introduce a group R¹⁰ onto a nitrogen of an appropriately protected compound, for example, protected compounds of formula I. Reductive alkylation is one particular method of introducing the group R¹⁰. Alternatively, R¹⁰ can be added to compounds by alkylation with an alkyl halide (such as, for example, methyl iodide, 4-(2-chloro-ethyl)-morpholine, 4-(3-chloro-propyl)-morpholine, 4-chloromethyl-tetrahydro-pyran, or 4-chloromethyl-N—BOC-piperidine) in solvents such as DMF or NMP using a base such as iPr₂EtN, Et₃N, Cs₂CO₃, or NaH, at temperatures ranging from 20-100° C., depending on the reagents. In the case of where R¹⁰ is introduced via reaction with a alkyl halide containing a BOC, PMB or THP protected nitrogen the group, could be removed during the final deprotection step and an alkyl group introduced onto the nitrogen by submitting the compound to standard methylation conditions such as reaction with MeI/K₂CO₃/DMF or by using reductive alkylation conditions such as CH₂O/MeOH/NaBH₃CN or using CH₂O/HCO₂H/H₂O. Alternatively, the protecting group could be removed selectively using TFA/CH₂Cl₂/anisole for BOC at an earlier stage (provided suitable protection groups are used elsewhere in the molecule, e.g. CH₂OCH₂Ph), followed by methylation using standard methylating conditions.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). In these circumstances a protected form of a compound of formula (I) may be produced by the procedures described above. In this case a deprotection step may be required to produce the final compound of formula (I). Deprotection of the protected form of a compound of formula (I) can be achieved using standard methods well know to those skilled in the art. Protecting groups and deprotection methods may be selected from standard groups and methods known in the art as discussed below.

A hydroxy group may be protected, for example, as an ether (—OR) or an ester (—OC(═O)R), for example, as: a t-butyl ether; a benzyl, benzhydryl(diphenylmethyl), or trityl(triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulfonyl(tosyl) and methanesulfonyl(mesyl) groups and benzyl groups such as apara-methoxybenzyl (PMB) group, or tetrahydropyran (THP). Alternatively, the amine can be protected by reaction with phthalic anhydride in acid under heat to give the isoindole-1,3-dione.

A carboxylic acid group may be protected as an ester for example, as: an C₁₋₇ alkyl ester (e.g., a methyl ester; a t-butyl ester); a C₁₋₇haloalkyl ester (e.g., a C₁₋₇ trihaloalkyl ester); a triC₁₋₇alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃). In some circumstances one of the protecting group described above may form part of the compound of Formula (I).

Novel chemical intermediates of the formula YYY form a further aspect of the invention. In particular, preferred novel intermediates are compounds of formula (IX) or (X), preferably (IX), and salts, solvates, esters or N-oxides thereof.

A further aspect of the invention is a process for the preparation of a compound of formula (I) as defined herein, which process comprises:
(i) the reaction of a compound of the formula (XIII) with R³—Y—CO₂H, or reactive derivative thereof and thereafter removing any protecting groups present; or

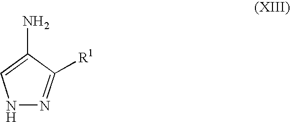

wherein R¹, R², and R³ are as defined herein; and optionally thereafter converting one compound of the formula (I) into another compound of the formula (I).

Methods of Purification

The compounds may be isolated and purified by a number of methods well known to those skilled in the art and examples of such methods include chromatographic techniques such as column chromatography (e.g. flash chromatography) and HPLC. Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described in the experimental section below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

Recrystallisation

Methods of recrystallisation of compounds of formula (I) and salts thereof can be carried out by methods well known to the skilled person—see for example (P. Heinrich Stahl (Editor), Camille G. Wermuth (Editor), ISBN: 3-90639-026-8, Handbook of Pharmaceutical Salts: Properties, Selection, and Use, Chapter 8, Publisher Wiley-VCH). Products obtained from an organic reaction are seldom pure when isolated directly from the reaction mixture. If the compound (or a salt thereof) is solid, it may be purified and/or crystallized by recrystallisation from a suitable solvent. A good recrystallisation solvent should dissolve a moderate quantity of the substance to be purified at elevated temperatures but only a small quantity of the substance at lower temperature. It should dissolve impurities readily at low temperatures or not at all. Finally, the solvent should be readily removed from the purified product. This usually means that it has a relatively low boiling point and a person skilled in the art will know recrystallising solvents for a particular substance, or if that information is not available, test several solvents. To get a good yield of purified material, the minimum amount of hot solvent to dissolve all the impure material is used. In practice, 3-5% more solvent than necessary is used so the solution is not saturated. If the impure compound contains an impurity which is insoluble in the solvent it may then be removed by filtration and then allowing the solution to crystallise. In addition, if the impure compound contains traces of coloured material that are not native to the compound, it may be removed by adding a small amount of decolorizing charcoal to the hot solution, filtering it and then allowing it to crystallise. Usually crystallisation spontaneously occurs upon cooling the solution. If it is not, crystallisation may be induced by cooling the solution below room temperature or by adding a single crystal of pure material (a seed crystal). Recrystallisation can also be carried out and/or the yield optimized by the use of an anti-solvent. In this case, the compound is dissolved in a suitable solvent at elevated temperature, filtered and then an additional solvent in which the required compound has low solubility is added to aid crystallisation. The crystals are then typically isolated using vacuum filtration, washed and then dried, for example, in an oven or via desiccation.

Other examples of methods for crystallisation include crystallisation from a vapor, which includes an evaporation step for example in a sealed tube or an air stream, and crystallisation from melt (Crystallization Technology Handbook 2nd Edition, edited by A. Mersmann, 2001).

In particular the compound of formula (I) may subjected to recrystallisation (e.g. using 2-propanol or ethanol as the solvent) to increase the purity and to give a crystalline form.

Generally, the crystals obtained are analysed by an X-ray diffraction method such as X-ray powder diffraction (XRPD) or X-ray crystal diffraction.

Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof such as formulae (II) and (III) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Examples of these are described in R. G. Strickly, Solubilizing Excipients in oral and injectable formulations, Pharmaceutical Research, Vol 21(2) 2004, p 201-230. In addition, they may contain co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

A drug molecule that is ionizable can be solubilized to the desired concentration by pH adjustment if the drug's pKa is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of Formula (I) or acid addition salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

Pharmaceutical compositions of the present invention for parenteral injection can also comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If a compound is not stable in aqueous media or has low solubility in aqueous media, it can be formulated as a concentrate in organic solvents. The concentrate can then be diluted to a lower concentration in an aqueous system, and can be sufficiently stable for the short period of time during dosing. Therefore in another aspect, there is provided a pharmaceutical composition comprising a non aqueous solution composed entirely of one or more organic solvents, which can be dosed as is or more commonly diluted with a suitable IV excipient (saline, dextrose; buffered or not buffered) before administration (Solubilizing excipients in oral and injectable formulations, Pharmaceutical Research, 21(2), 2004, p 201-230). Examples of solvents and surfactants are propylene glycol, PEG300, PEG400, ethanol, dimethylacetamide (DMA), N-methyl-2-pyrrolidone (NMP, Pharmasolve), Glycerin, Cremophor EL, Cremophor RH 60 and polysorbate. Particular non aqueous solutions are composed of 70-80% propylene glycol, and 20-30% ethanol. One particular non aqueous solution is composed of 70% propylene glycol, and 30% ethanol. Another is 80% propylene glycol and 20% ethanol.Normally these solvents are used in combination and usually diluted at least 2-fold before IV bolus or IV infusion. The typical amounts for bolus IV formulations are ˜50% for Glycerin, propylene glycol, PEG300, PEG400, and ˜20% for ethanol. The typical amounts for IV infusion formulations are ˜15% for Glycerin, 3% for DMA, and ˜10% for propylene glycol, PEG300, PEG400 and ethanol.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The compounds of the inventions will generally be presented in unit dosage form and, as such, will typically contain sufficient compound to provide a desired level of biological activity. For example, a formulation intended for oral administration may contain from 1 picogram to 2 milligrams of active ingredient, e.g. from 1 nanogram to 2 milligrams of active ingredient. Within this range, particular sub-ranges of compound are 0.1 milligrams to 2 grams of active ingredient more usually from 10 milligrams to 1 gram, for example, 50 milligrams to 500 milligrams or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

Methods of Treatment

It is envisaged that the compounds of the formula (I) and sub-groups thereof such as formulae (II) and (III) and sub-groups thereof as defined herein will be useful in the prophylaxis or treatment of a range of disease states or conditions mediated by cyclin dependent kinases, glycogen synthase kinase-3 and Aurora kinases. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams such as 1 micrograms to 10 milligrams) per kilogram of bodyweight although higher or lower doses may be administered where required. Ultimately, the quantity of compound administered and the type of composition used will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example a neoplastic disease such as a cancer as hereinbefore defined. Examples of other therapeutic agents that may be administered together (whether concurrently or at different time intervals) with the compounds of the formula (I) include but are not limited to topoisomerase inhibitors, alkylating agents, antimetabolites, DNA binders, microtubule inhibitors (tubulin targeting agents), monoclonal antibodies or signal transduction inhibitors. For the case of CDK or Aurora inhibitors combined with other therapies, the two or more treatments may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through their common general knowledge the dosing regimes and combination therapies to use.

Methods of Diagnosis

Prior to administration of a compound of the formula (I), a patient may be screened to determine whether a disease or condition from which the patient is or may be suffering is one which would be susceptible to treatment with a compound having activity against Aurora and/or cyclin dependent kinases.

For example, a biological sample taken from a patient may be analysed to determine whether a condition or disease, such as cancer, that the patient is or may be suffering from is one which is characterised by a genetic abnormality or abnormal protein expression which leads to over-activation of CDKs or to sensitisation of a pathway to normal CDK activity. Examples of such abnormalities that result in activation or sensitisation of the CDK2 signal include up-regulation of cyclin E, (Harwell R M,

Mull B B, Porter D C, Keyomarsi K.; J Biol Chem. 2004 Mar. 26; 279(13):12695-705) or loss of p21 or p27, or presence of CDC4 variants (Rajagopalan H, Jallepalli P V, Rago C, Velculescu V E, Kinzler K W, Vogelstein B, Lengauer C.; Nature. 2004 Mar. 4; 428(6978):77-81). Tumours with mutants of CDC4 or up-regulation, in particular over-expression, of cyclin E or loss of p21 or p27 may be particularly sensitive to CDK inhibitors. Alternatively or in addition, a biological sample taken from a patient may be analysed to determine whether a condition or disease, such as cancer, that the patient is or may be suffering from is one which is characterised by upregulation of Aurora kinase and thus may be particularly to Aurora inhibitors. The term up-regulation includes elevated expression or over-expression, including gene amplification (i.e. multiple gene copies) and increased expression by a transcriptional effect, and hyperactivity and activation, including activation by mutations.

Thus, the patient may be subjected to a diagnostic test to detect a marker characteristic of over-expression, up-regulation or activation of Aurora kinase or the patient may be subjected to a diagnostic test to detect a marker characteristic of up-regulation of cyclin E, or loss of p21 or p27, or presence of CDC4 variants. The term diagnosis includes screening. By marker we include genetic markers including, for example, the measurement of DNA composition to identify mutations of Aurora or CDC4. The term marker also includes markers which are characteristic of up regulation of Aurora or cyclin E, including enzyme activity, enzyme levels, enzyme state (e.g. phosphorylated or not) and mRNA levels of the aforementioned proteins. Tumours with upregulation of cyclin E, or loss of p21 or p27 may be particularly sensitive to CDK inhibitors. Tumours may preferentially be screened for upregulation of cyclin E, or loss of p21 or p27 prior to treatment. Thus, the patient may be subjected to a diagnostic test to detect a marker characteristic of up-regulation of cyclin E, or loss of p21 or p27.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found, see Ewart-Toland et al., (Nat Genet. 2003 August; 34(4):403-12), that individuals forming part of the sub-population possessing the I1e31 variant of the STK gene (the gene for Aurora kinase A) may have an increased susceptibility to certain forms of cancer. It is envisaged therefore that such individuals suffering from cancer will benefit from the administration of compounds having Aurora kinase inhibiting activity. A patient suffering from, or suspected of suffering from, a cancer may therefore be screened to determine whether he or she forms part of the Ile31 variant sub-population. In addition, it has been found, Rajagopalan et al (Nature. 2004 Mar. 4; 428(6978):77-81), that there were mutations present in CDC4 (also known as Fbw7 or Archipelago) in human colorectal cancers and endometrial cancers (Spruck et al, Cancer Res. 2002 Aug. 15; 62(16):4535-9). Identification of individual carrying a mutation in CDC4 may mean that the patient would be particularly suitable for treatment with a CDK inhibitor. Tumours may preferentially be screened for presence of a CDC4 variant prior to treatment. The screening process will typically involve direct sequencing, oligonucleotide microarray analysis, or a mutant specific antibody.

Tumours with activating mutants of Aurora or up-regulation of Aurora including any of the isoforms thereof, may be particularly sensitive to Aurora inhibitors. Tumours may preferentially be screened for up-regulation of Aurora or for Aurora possessing the Ile31 variant prior to treatment (Ewart-Toland et al., Nat Genet. 2003 August; 34(4):403-12). Ewart-Toland et al identified a common genetic variant in STK15 (resulting in the amino acid substitution F31I) that is preferentially amplified and associated with the degree of aneuploidy in human colon tumors. These results are consistent with an important role for the Ile31 variant of STK15 in human cancer susceptibility. In particular, this polymorphism in Aurora A has been suggested to be a genetic modifier fir developing breast carcinoma (Sun et al, Carcinogenesis, 2004, 25(11), 2225-2230).

The aurora A gene maps to the chromosome 20q13 region that is frequently amplified in many cancers e.g. breast, bladder, colon, ovarian, pancreatic. Patients with a tumour that has this gene amplification might be particularly sensitive to reatments targeting aurora kinase inhibition

Methods of identification and analysis of mutations and up-regulation of protein e.g. Aurora isoforms and chromosome 20q13 amplification are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of cyclin E, or loss of p21 or p27, or detection of CDC4 variants, Aurora up-regulation and mutants of Aurora could be applicable in the present case.

Therefore, all of these techniques could also be used to identify tumours particularly suitable for treatment with the compounds of the invention.

Tumours with mutants of CDC4 or up-regulation, in particular over-expression, of cyclin E or loss of p21 or p27 may be particularly sensitive to CDK inhibitors. Tumours may preferentially be screened for up-regulation, in particular over-expression, of cyclin E (Harwell R M, Mull B B, Porter D C, Keyomarsi K.; J Biol Chem. 2004 Mar. 26; 279(13):12695-705) or loss of p21 or p27 or for CDC4 variants prior to treatment (Rajagopalan H, Jallepalli P V, Rago C, Velculescu V E, Kinzler K W, Vogelstein B, Lengauer C.; Nature. 2004 Mar. 4; 428(6978):77-81).

Patients with mantle cell lymphoma (MCL) could be selected for treatment with a compound of the invention using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(11;14)(q13;q32) translocation. Over-expression of cyclin D1 mRNA, found in mantle cell lymphoma (MCL), is a critical diagnostic marker. Yatabe et al (Blood. 2000 Apr. 1; 95(7):2253-61) proposed that cyclin D1-positivity should be included as one of the standard criteria for MCL, and that innovative therapies for this incurable disease should be explored on the basis of the new criteria. Jones et al (J Mol Diagn. 2004 May; 6(2):84-9) developed a real-time, quantitative, reverse transcription PCR assay for cyclin D1 (CCND1) expression to aid in the diagnosis of mantle cell lymphoma (MCL). Howe et al (Clin Chem. 2004 January; 50(1):80-7) used real-time quantitative RT-PCR to evaluate cyclin D1 mRNA expression and found that quantitative RT-PCR for cyclin D1 mRNA normalized to CD19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer (Harwell et al, Cancer Res, 2000, 60, 481-489). Therefore breast cancer may in particular be treated with a CDK inhibitor as provided herein.

Antifungal Use

In a further aspect, the invention provides the use of the compounds of the formula (I) and sub-groups thereof as defined herein as hereinbefore defined as antifungal agents.

The compounds of the formula (I) and sub-groups thereof such as formulae (II) and (III) and sub-groups thereof as defined herein may be used in animal medicine (for example in the treatment of mammals such as humans), or in the treatment of plants (e.g. in agriculture and horticulture), or as general antifungal agents, for example as preservatives and disinfectants.

In one embodiment, the invention provides a compound of the formula (I) and sub-groups thereof such as formulae (II) and (III) and sub-groups thereof as defined herein as hereinbefore defined for use in the prophylaxis or treatment of a fungal infection in a mammal such as a human.

Also provided is the use of a compound of the formula (I) and sub-groups thereof such as formulae (II) and (III) and sub-groups thereof as defined herein for the manufacture of a medicament for use in the prophylaxis or treatment of a fungal infection in a mammal such as a human.

For example, compounds of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The compounds of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of the formula (I) and sub-groups thereof such as formulae (II) and (III) and sub-groups thereof as defined herein together with an agriculturally acceptable diluent or carrier.

The invention further provides a method of treating an animal (including a mammal such as a human), plant or seed having a fungal infection, which comprises treating said animal, plant or seed, or the locus of said plant or seed, with an effective amount of a compound of the formula (I) and sub-groups thereof such as formulae (II) and (III) and sub-groups thereof as defined herein.

The invention also provides a method of treating a fungal infection in a plant or seed which comprises treating the plant or seed with an antifungally effective amount of a fungicidal composition containing a compound of the formula (I) and sub-groups thereof such as formulae (II) and (III) and sub-groups thereof as hereinbefore defined.

Differential screening assays may be used to select for those compounds of the present invention with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocaidiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus arrhizus, Rhizopus oryzae, Absidia corymbifera, Absidia ramosa, or Mucorpusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fungus and the appropriate M.I.C. value is noted. Alternatively, a turbidity assay in liquid cultures can be performed and a protocol outlining an example of this assay can be found in Example 56.

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed by monitoring the growth of the fungal infection in groups of treated and untreated mice (by histology or by retrieving fungi from the infection). The activity may be measured in terms of the dose level at which the compound provides 50% protection against the lethal effect of the infection (PD₅₀).

For human antifungal use, the compounds of the formula (I) and sub-groups thereof such as formulae (II) and (III) and sub-groups thereof as defined herein can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level of the antifungal compounds of the invention can be from 0.01 to 10 mg/kg (in divided doses), depending on inter alia the potency of the compounds when administered by either the oral or parenteral route. Tablets or capsules of the compounds may contain, for example, from 5 mg to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal compounds can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulations of the compounds of the invention in insecticides, such as for use in management of insects like the fruit fly.

In yet another embodiment, certain of the subject CDK inhibitors can be selected on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

For agricultural and horticultural purposes the compounds of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they can be manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt. % of the active ingredient. For field use, likely application rates of the active ingredient may be from 50 to 5000 g/hectare.

The invention also contemplates the use of the compounds of the formula (I) and sub-groups thereof such as formulae (II) and (III) and sub-groups thereof as defined herein in the control of wood decaying fungi and in the treatment of soil where plants grow, paddy fields for seedlings, or water for perfusion. Also contemplated by the invention is the use of the compounds of the formula (I) and sub-groups thereof such as formulae (II) and (III) and sub-groups thereof as defined herein to protect stored grain and other non-plant loci from fungal infestation.

### EXAMPLES

The invention will now be illustrated, but not limited, by reference to the specific embodiments described in the following examples.

In the examples, the following abbreviations are used.

AcOH acetic acid

BOC tert-butyloxycarbonyl

CDI 1,1-carbonyldiimidazole

DMAW90 Solvent mixture: DCM: MeOH, AcOH, H₂O (90:18:3:2)

DMAW120 Solvent mixture: DCM: MeOH, AcOH, H₂O (120:18:3:2)

DMAW240 Solvent mixture: DCM: MeOH, AcOH, H₂O (240:20:3:2)

DCM dichloromethane

DMF dimethylformamide

DMSO dimethyl sulphoxide

EDC 1-ethyl-3-(3′-dimethylaminopropyl)-carbodiimide

Et₃N triethylamine

EtOAc ethyl acetate

Et₂O diethyl ether

HOAt 1-hydroxyazabenzotriazole

HOBt 1-hydroxybenzotriazole

MeCN acetonitrile

MeOH methanol

SiO₂ silica

TBTU N,N,N′,N′-tetramethyl-O-(benzotriazol-1-yl)uronium tetrafluoroborate

THF tetrahydrofuran

Analytical LC-MS System and Method Description

In the examples, the compounds prepared were characterised by liquid chromatography and mass spectroscopy (LC-MS) using the systems and operating conditions set out below. Where atoms with different isotopes are present, and a single mass quoted, the mass quoted for the compound is the monoisotopic mass (i.e. ³⁵Cl; ⁷⁹Br etc.). Several systems were used, as described below, and these were set up to run under closely similar operating conditions. The operating conditions used are also described below.

Waters Platform LC-MS system:

HPLC System: Waters 2795

Mass Spec Detector: Micromass Platform LC

PDA Detector: Waters 2996 PDA

Analytical Acidic conditions:

Eluent A: H₂O (0.1% Formic Acid)

Eluent B: CH₃CN (0.1% Formic Acid)

Gradient: 5-95% eluent B over 3.5 minutes

Flow: 0.8 ml/min

Column: Phenmenex Synergi 4μ MAX-RP 80A, 2.0×50 mm

Analytical Basic Conditions:

Eluent A: H₂O (10 mM NH₄HCO₃ buffer adjusted to pH=9.2 with NH₄OH)

Eluent B: CH₃CN

Gradient: 05-95% eluent B over 3.5 minutes

Flow: 0.8 ml/min

Column: Phenomenex Luna C18(2) 5 μm 2.0×50 mm

Analytical Polar Conditions:

Eluent A: H₂O (0.1% Formic Acid)

Eluent B: CH₃CN (0.1% Formic Acid)

Gradient: 00-50% eluent B over 3 minutes

Flow: 0.8 ml/min

Column: Phenomenex Synergi 4μ MAX-RP 80A, 2.0×50 mm

Analytical Lipophilic Conditions:

Eluent A: H₂O (0.1% Formic Acid)

Eluent B: CH₃CN (0.1% Formic Acid)

Gradient: 55-95% eluent B over 3.5 minutes

Flow: 0.8 ml/min

Column: Phenomenex Synergi 4μ MAX-RP 80A, 2.0×50 mm

Analytical Long Acidic Conditions:

Eluent A: H₂O (0.1% Formic Acid)

Eluent B: CH₃CN (0.1% Formic Acid)

Gradient: 05-95% eluent B over 15 minutes

Flow: 0.4 ml/min

Column: Phenomenex Synergi 4μ, MAX-RP 80A, 2.0×150 mm

Analytical Long Basic Conditions:

Fluent A: H₂O (10 mM NH₄HCO₃ buffer adjusted to pH=9.2 with NH₄OH)

Eluent B: CH₃CN

Gradient: 05-95% eluent B over 15 minutes

Flow: 0.8 ml/min

Column: Phenomenex Luna C18(2) 5 μm 2.0×50 mm

Platform MS Conditions:

Capillary voltage: 3.6 kV (3.40 kV on ES negative)

Cone voltage: 25 V

Source Temperature: 120 ° C.

Scan Range: 100-800 amu

Ionisation Mode: ElectroSpray Positive or
ElectroSpray Negative or
ElectroSpray Positive & Negative

Waters Fractionlynx LC-MS system:

HPLC System: 2767 autosampler—2525 binary gradient pump

Mass Spec Detector: Waters ZQ

PDA Detector: Waters 2996 PDA

Analytical Acidic Conditions:

Eluent A: H₂O (0.1% Formic Acid)

Eluent B: CH₃CN (0.1% Formic Acid)

Gradient: 5-95% eluent B over 4 minutes

Flow: 2.0 ml/min

Column: Phenomenex Synergi 4μ, MAX-RP 80A, 4.6×50 mm

Analytical Polar Conditions:

Eluent A: H₂O (0.1% Formic Acid)

Eluent B: CH₃CN (0.1% Formic Acid)

Gradient: 00-50% eluent B over 4 minutes

Flow: 2.0 ml/min

Column: Phenomenex Synergi 4μ MAX-RP 80A, 4.6×50 mm

Analytical Lipophilic Conditions:

Eluent A: H₂O (0.1% Formic Acid)

Eluent B: CH₃CN (0.1% Formic Acid)

Gradient: 55-95% eluent B over 4 minutes

Flow: 2.0 ml/min

Column: Phenomenex Synergi 4μ MAX-RP 80A, 4.6×50 mm

Fractionlynx MS Conditions:

Capillary voltage: 3.5 kV (3.2 kV on ES negative)

Cone voltage: 25 V (30 V on ES negative)

Source Temperature: 120 ° C.

Scan Range: 100-800 amu

Ionisation Mode: ElectroSpray Positive or
ElectroSpray Negative or
ElectroSpray Positive & Negative

Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

Two such system for purifying compounds via preparative LC-MS are described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

Preparative LC-MS Systems:

Waters Fractionlynx System:

Hardware:

2767 Dual Loop Autosampler/Fraction Collector

2525 preparative pump

CFO (column fluidic organiser) for column selection

RMA (Waters reagent manager) as make up pump

Waters ZQ Mass Spectrometer

Waters 2996 Photo Diode Array detector

Waters ZQ Mass Spectrometer

Software:

Masslynx 4.0

Waters MS Running Conditions:

Capillary voltage: 3.5 kV (3.2 kV on ES Negative)

Cone voltage: 25 V

Source Temperature: 120° C.

Multiplier: 500 V

Scan Range: 125-800 amu
Ionisation Mode: ElectroSpray Positive or
ElectroSpray Negative

Agilent 1100 LC-MS Preparative System:

Hardware:

Autosampler: 1100 series “prepALS”

Pump: 1100 series “PrepPump” for preparative flow gradient and 1100 series

“QuatPump” for pumping modifier in prep flow

UV detector: 1100 series “MWD” Multi Wavelength Detector

MS detector: 1100 series “LC-MSD VL”

Fraction Collector: 2× “Prep-FC”

Make Up pump: “Waters RMA”

Agilent Active Splitter

Software:

Chemstation: Chem32

Agilent MS Running Conditions:

Capillary voltage: 4000 V (3500 V on ES Negative)

Fragmentor/Gain: 150/l

Drying gas flow: 13.0 L/min

Gas Temperature: 350° C.

Nebuliser Pressure: 50 psig

Scan Range: 125-800 amu

Ionisation Mode: ElectroSpray Positive or
ElectroSpray Negative

Chromatographic Conditions:

Columns:

1. Low pH chromatography:

Phenomenex Synergy MAX-RP, 10μ, 100×21.2 mm (alternatively used Thermo Hypersil-Keystone HyPurity Aquastar, 5μ, 100×21.2 mm for more polar compounds)

2. High pH chromatography:

Phenomenex Luna C18 (2), 10μ, 100×21.2 mm (alternatively used Phenomenex Gemini, 5μ, 100×21.2 mm)

Eluents:

1. Low pH chromatography:

Solvent A: H₂O+0.1% Formic Acid, pH˜1.5

Solvent B: CH₃CN+0.1% Formic Acid

2. High pH chromatography:

Solvent A: H₂0+10 mM NH₄HCO₃+NH4OH, pH=9.2

Solvent B: CH₃CN

3. Make up solvent:

MeOH+0.2% Formic Acid (for both chromatography type)

Methods:

According to the analytical trace the most appropriate preparative chromatography type was chosen. A typical routine was to run an analytical LC-MS (see above) using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min

Gradient: Generally all gradients had an initial 0.4 min step with 95% A+5% B.

Then according to analytical trace a 3.6 min gradient was chosen in order to achieve good separation (e.g. from 5% to 50% B for early retaining compounds; from 35% to 80% B for middle retaining compounds and so on)

Wash: 1.2 minute wash step was performed at the end of the gradient

Re-equilibration: 2.1 minutes re-equilibration step was ran to prepare the system for the next run

Make Up flow rate: 1 ml/min

Solvent:

All compounds were usually dissolved in 100% MeOH or 100% DMSO

From the information provided someone skilled in the art could purify the compounds described herein by preparative LC-MS.

### Example 1

### Synthesis of 2,6-difluoro-N-(3-pyridin-3-yl-1H-pyrazol-4-yl)-benzamide

### 1A. Synthesis of 3-(1H-pyrazol-3-yl)-pyridine

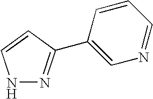

A mixture of 3-dimethylamino-1-pyridin-3-yl-propenone (1.0 g, 5.7 mmol) and hydrazine monohydrate (0.3 g) in 2-methoxyethanol (15 mL) was heated at reflux under an atmosphere of nitrogen for 3 h, then reduced in vacuo to give the title compound (806 mg) as an orange gum.

### 1B. Synthesis of 3-(4-nitro-1H-pyrazol-3-yl)-pyridine

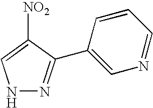

To a mixture of 3-(1H-pyrazol-3-yl)-pyridine (650 mg, 4.5 mmol) in concentrated sulfuric acid (15 mL) was carefully added fuming nitric acid (5 mL). The mixture was stirred at ambient temperature for 41 h, then 100° C. for 1 h. The mixture was then poured over ice, neutralised using sodium carbonate and the solid formed collected by filtration, washing with water, and dried in vacuo, azeotroping with toluene to give the title compound (752 mg) as a white solid.

### 1C. Synthesis of 3-pyridin-3-yl-1H-pyrazol-4-ylamine

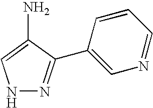

A mixture 3-(4-nitro-1H-pyrazol-3-yl)-pyridine (400 mg, 2.1 mmol) and 10% Pd/C (60 mg) in MeOH-DMF (5:1, 12 mL) was shaken under an atmosphere of hydrogen for 4 h, filtered through a plug of Celite and reduced in vacuo to give the title compound (306 mg) as a brown solid.

### 1D. Synthesis of 2,6-difluoro-N-(3-pyridin-3-yl-1H-pyrazol-4-yl)-benzamide

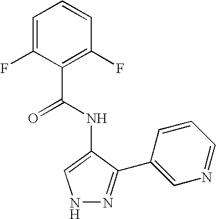

A mixture of 3-pyridin-3-yl-1H-pyrazol-4-ylamine (300 mg, 1.9 mmol), 2,6-difluorobenzoic acid (269 mg, 1.7 mmol), EDC (384 mg, 2.0 mmol) and HOBt (270 mg, 2.0 mmol) in DMF (8 mL) was stirred at ambient temperature for 48 h, reduced in vacuo and the residue partitioned between EtOAc and saturated aqueous NaHCO₃. The layers were separated and the organic portion was washed with brine, dried (MgSO₄) and reduced in vacuo. The residue was purified by preparative LC/MS to give the title compound (98 mg) as a beige solid. (LC/MS: Rₜ 1.95, M+H⁺301.06).

### Example 2

### Synthesis of N-[3-(1,3-dioxo-1,3-dihydro-isoindol-2-yl)-1H-pyrazol-4-yl]-2,6-difluoro-benzamide

### 2A. Synthesis of 2-(4-nitro-1H-pyrazol-3-yl)-isoindole-1,3-dione

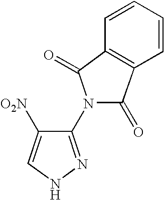

A mixture of 3-amino-4-nitropyrazole (640 mg, 5 mmol) and phthalic anhydride (889 mg, 6 mmol) in glacial AcOH (20 mL) was heated at reflux for 16 h. The precipitate formed was collected by filtration, washing with methanol, and dried in vacuo to give the title compound (575 mg) as a white solid.

### 2B. Synthesis of 2-(4-amino-1H-pyrazol-3-yl)-isoindole-1,3-dione

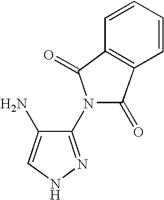

A mixture 2-(4-nitro-1H-pyrazol-3-yl)-isoindole-1,3-dione (250 mg, 1 mmol) and 10% Pd/C (50 mg) in DMF (10 mL) was shaken under an atmosphere of hydrogen for 20 h, filtered through a plug of Celite and reduced in vacuo to give a mixture containing the title compound.

2C. Synthesis of N-[3-(1,3-dioxo-1,3-dihydro-isoindol-2-yl)-1H-pyrazol-4-yl]-2,6-difluoro-benzamide

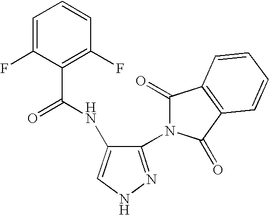

A mixture of the step 2 product, 2,6-difluorobenzoic acid (111 mg, 0.7 mmol), EDC (192 mg, 1.0 mmol) and HOBt (135 mg, 1.0 mmol) in DMF (5 mL) was stirred at ambient temperature for 6 h, reduced in vacuo and the residue partitioned between EtOAc and saturated aqueous NaHCO₃. The layers were separated and the organic portion was washed with brine, dried (MgSO₄) and reduced in vacuo. The residue was purified by column chromatography (P.E.-EtOAc (1:0-0:1)) to give the title compound (158 mg) as an off-white solid. (LC/MS: Rₜ 2.45, [M+H]⁺368.98).

### Example 3

Synthesis of 2,6-difluoro-N-[3-(5-oxo-4,5-dihydro-1H-[1,2,4]triazol-3-yl)-1H-pyrazol-4-yl]-benzamide

### 3A. Synthesis of 4-Nitro-1H-pyrazole-3-carboxylic acid ethyl ester

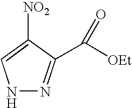

Thionyl chloride (2.90 ml, 39.8 mmol) was slowly added to a mixture of 4-nitro-3-pyrazolecarboxylic acid (5.68 g, 36.2 mmol) in EtOH (100 ml) at ambient temperature and the mixture stirred for 48 h. The mixture was reduced in vacuo and dried through azeotrope with toluene to afford 4-nitro-1H-pyrazole-3-carboxylic acid ethyl ester as a white solid (6.42 g, 96%). (¹H NMR (400 MHz, DMSO-d₆) δ 14.4 (s, 1H), 9.0 (s, 1H), 4.4 (q, 2H), 1.3 (t, 3H)).

### 3B. Synthesis of 4-Amino-1H-pyrazole-3-carboxylic acid ethyl ester

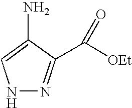

A mixture of 4-nitro-1H-pyrazole-3-carboxylic acid ethyl ester (6.40 g, 34.6 mmol) and 10% Pd/C (650 mg) in EtOH (150ml) was stirred under an atmosphere of hydrogen for 20 h. The mixture was filtered through a plug of Celite, reduced in vacuo and dried through azeotrope with toluene to afford 4-amino-1H-pyrazole-3-carboxylic acid ethyl ester as a pink solid (5.28 g, 98%). (¹H NMR (400 MHz, DMSO-d₆) δ 12.7 (s, 1H), 7.1 (s, 1H), 4.8 (s, 2H), 4.3 (q, 2H), 1.3 (t, 3H)).

### 3C. Synthesis of 4-(2,6-Difluoro-benzoylamino)-1H-pyrazole-3-carboxylic acid ethyl ester

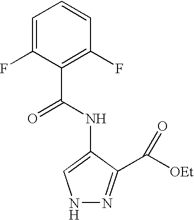

A mixture of 2,6-difluorobenzoic acid (6.32 g, 40.0 mmol), 4-amino-1H-pyrazole-3-carboxylic acid ethyl ester (5.96 g, 38.4 mmol), EDC (8.83 g, 46.1 mmol) and HOBt (6.23 g, 46.1 mmol) in DMF (100 ml) was stirred at ambient temperature for 6 h. The mixture was reduced in vacuo, water added and the solid formed collected by filtration and air-dried to give 4-(2,6-difluoro-benzoylamino)-1H-pyrazole-3-carboxylic acid ethyl ester as the major component of a mixture (15.3 g). (LC/MS: Rₜ 3.11, [M+H]⁺295.99).

### 3D. Synthesis of 4-(2,6-Difluoro-benzoylamino)-1H-pyrazole-3-carboxylic acid

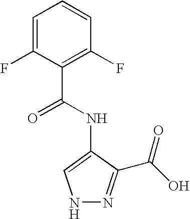

A mixture of 4-(2,6-difluoro-benzoylamino)-1H-pyrazole-3-carboxylic acid ethyl ester (10.2 g) in 2 M aqueous NaOH/MeOH (1:1, 250 ml) was stirred at ambient temperature for 14 h. Volatile materials were removed in vacuo, water (300 ml) added and the mixture taken to pH 5 using 1M aqueous HCl. The resultant precipitate was collected by filtration and dried through azeotrope with toluene to afford 4-(2,6-difluoro-benzoylamino)-1H-pyrazole-3-carboxylic acid as a pink solid (5.70 g). (LC/MS: Rₜ 2.33, [M+H]⁺267.96).

### 3E. Synthesis of 4-(2,6-difluoro-benzoylamino)-1H-pyrazole-3-carboxylic acid methyl ester

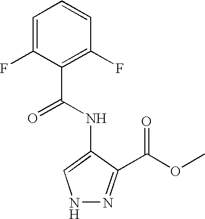

A mixture of 4-(2,6-difluoro-benzoylamino)-1H-pyrazole-3-carboxylic acid (6.0 g, 23 mmol) and thionyl chloride (4.8 mL) in methanol (100 mL) was heated at reflux for 28 h, allowed to cool and reduced in vacuo azeotroping with toluene (×3) to give the title compound (6.24 g) as a pale yellow solid.

### 3F. Synthesis of 2,6-difluoro-N-(3-hydrazinocarbonyl-1H-pyrazol-4-yl)-benzamide

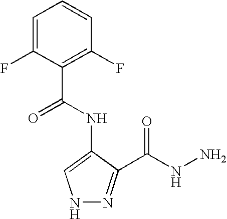

A mixture of 4-(2,6-difluoro-benzoylamino)-1H-pyrazole-3-carboxylic acid methyl ester (2.0 g) and hydrazine monohydrate (1.8 mL) in ethanol (30 mL) was heated at reflux for 1 h, allowed to cool and reduced in vacuo azeotroping with toluene (×3) to give the title compound (2.1 g) as a light brown solid.

### 3G. Synthesis of 2,6-difluoro-N-{3-[4-(4-methoxy-benzyl)-5-oxo-4,5-dihydro-1H-1,2,4]triazol-3-yl]-1H-pyrazol-4-yl}-benzamide

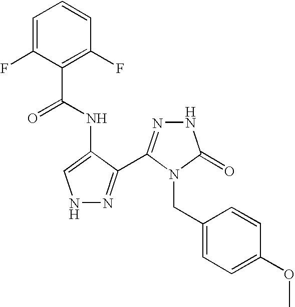

A mixture of 2,6-difluoro-N-(3-hydrazinocarbonyl-1H-pyrazol-4-yl)-benzamide (888 mg) and para-methoxybenzylisocyanate (0.5 mL) in anhydrous THF (25 mL) was stirred under a nitrogen atmosphere at ambient temperature for 6 h. The mixture was reduced in vacuo to give the intermediate hydrazide (1.71 g) as a beige solid. A mixture of the hydrazide (1.5 g) in 2 M aqueous NaOH solution (45 mL) was heated at reflux for 4 h, allowed to cool to ambient and then poured into saturated aqueous NH₄Cl (200 mL). The solid formed was collected by filtration and dried through azeotrope with toluene to give the title compound (1.19 g) as an off-white solid.

### 3H. Synthesis of 2,6-difluoro-N-[3-(5-oxo-4,5-dihydro-1H-[1,2,4]triazol-3-yl)-1H-pyrazol-4-yl]-benzamide

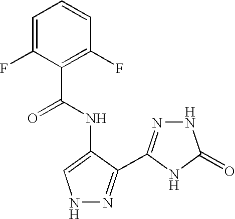

A mixture of 2,6-difluoro-N-{3-[4-(4-methoxy-benzyl)-5-oxo-4,5-dihydro-1H-[1,2,4]triazol-3-yl]-1H-pyrazol-4-yl}-benzamide (300 mg), anisole (300 □L) and trifluoroacetic acid (3 mL) was heated in the microwave (90° C., 100 W, 30 min). The mixture was reduced in vacuo and triturated with ether leaving a white solid which was then purified by preparative LC/MS to give the title compound (8 mg) as a white solid. (LC/MS: Rₜ 2.02, [M+H]⁺306.95).

### Example 4

### Synthesis of 2,6-Difluoro-N-[3-(2-methyl-thiazol-4-yl)-1H-pyrazol-4-yl]-benzamide

### 4A. Synthesis of 4-Nitro-1H-pyrazole-3-carboxylic acid methyl ester

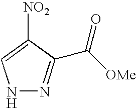

Thionyl chloride (3.8 ml, 52.5 mmol) was added cautiously to a stirred, ice-cold mixture of 4-nitropyrazole-3-carboxylic acid (7.5 g, 47.7 mmol) in MeOH (150 ml), the mixture stirred at ambient temperature for 1 hour then heated at reflux for 3 hours. The reaction mixture was cooled, evaporated in vacuo then azeotroped with toluene to give 4-nitro-1H-pyrazole-3-carboxylic acid ethyl ester (8.8 g).

### 4B. Synthesis of 4-Nitro-1-(tetrahydro-pyran-2-yl)-1H-pyrazole-3-carboxylic acid methyl ester

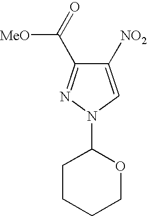

A suspension of 4-nitro-1H-pyrazole-3-carboxylic acid methyl ester (5 g, 29.24 mmol) and p-toluene sulphonic acid (555 mg, 2.92 mmol) in chloroform (100 ml) at 0° C. was treated with 3,4-dihydropyran (4 ml, 43.8 mmol) dropwise. The reaction mixture was allowed to warm to ambient temperature, and then stirred for a further 2 hours. The reaction mixture was diluted with Et₂O, washed sequentially with saturated NaHCO₃ solution and brine. The organic portion was dried (MgSO₄), filtered and evaporated in vacuo. The residue was purified by flash chromatography [silica, EtOAc/Petrol (1:2)] to give 4-nitro-1-(tetrahydro-pyran-2-yl)-1H-pyrazole-3-carboxylic acid methyl ester (7.1 g, 95%) as a colourless oil. (LC/MS: Rₜ 2.86, [M+H]⁺256.00).

### 4C. Synthesis of 4-amino-1-(tetrahydro-pyran-2-yl)-1H-pyrazole-3-carboxylic acid methyl ester

To a stirred solution of 4-nitro-1-(tetrahydro-pyran-2-yl)-1H-pyrazole-3-carboxylic acid methyl ester (16.0 g, 62.75 mmoles, example 14B) and ammonium formate (39.6 g, 627.45 mmoles) and in ethanol (200 ml) and water (20 ml) under nitrogen was added palladium on carbon (10%, 0.8 g). The reaction mixture was heated at 50° C. for 2 hours. The suspension was filtered through celite, and the filtrate was partitioned between EtOAc and water. The organic portion was dried (MgSO₄) to give 4-amino-1-(tetrahydro-pyran-2-yl)-1H-pyrazole-3-carboxylic acid methyl ester as a yellow oil (12.5 g, 89%). (LC/MS: Rₜ 1.84, [M+H]⁺226.06).

### 4D. Synthesis of 4-(2,6-difluoro-benzoylamino)1-(tetrahydro-pyran-2-yl-1H-pyrazole-3-carboxylic acid methyl ester

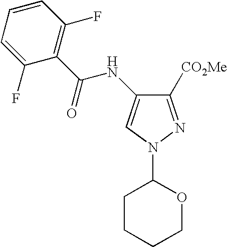

A solution of 4-amino-1-(tetrahydro-pyran-2-yl)-1H-pyrazole-3-carboxylic acid methyl ester (12.5 g, 55.56 mmoles), EDC (18.78 g, 97.96 mmoles), HOBt (13.00 g, 96.30 mmoles) and 2,6-difluorobenzoic acid (12.8 g, 81.01 mmoles) in dichloromethane was stirred at ambient temperature for 24 hours, and then partitioned between EtOAc and NaOH solution (2N). The organic portion was dried (MgSO₄), filtered and evaporated in vacuo. The residue was purified [Biotage SP4, 3×40M, flow rate 40 ml/min, gradient 3:7 EtOAc/Petrol to 2:1 EtOAc/ Petrol] to give 4-(2,6-difluoro-benzoylamino)1-(tetrahydro-pyran-2-yl-1H-pyrazole-3-carboxylic acid methyl ester as a white solid (11.3 g, 56%). (LC/MS: Rₜ 3.10, [M+H]⁺366.19).

### 4E. Synthesis of 2,6-difluoro-N-[3-hydroxymethyl-1-(tetrahydro-pyran-2-yl)-1H-pyrazol-4-yl]-benzamide

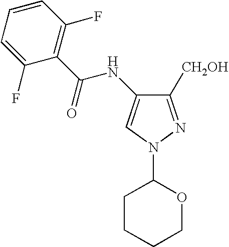

A stirred solution of 4-(2,6-difluoro-benzoylamino)1-(tetrahydro-pyran-2-yl-1H-pyrazole-3-carboxylic acid methyl ester (11.3 g, 30.96 mmoles) in THF (500 ml) under nitrogen at −78° C. was treated dropwise with a solution of diisobutylaluminium hydride in THF (310 ml, 1M). The reaction mixture was stirred at −78° C. for 1 hour and then warmed to 0° C. in an ice-water bath. A saturated aqueous solution of sodium sulphate (300 ml) was added to the reaction mixture. The suspension was filtered through celite. The filtrate was partitioned between EtOAc and brine. The organic portion was dried (MgSO₄), filtered and evaporated in vacuo to give 2,6-difluoro-N-[3-hydroxymethyl-1-(tetrahydro-pyran-2-yl)-1H-pyrazol-4-yl-benzamide as a white solid (10.14 g, 97%). (LC/MS: Rₜ 2.34, [M+H]⁺338.03).

### 4F. Synthesis of 2,6-difluoro-N-[3-formyl-1-(tetrahydro-pyran-2-yl)-1H-pyrazol-4-yl]-benzamide

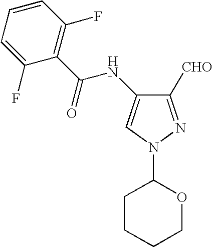

To a stirred solution of 2,6-difluoro-N-[3-hydroxymethyl-1-(tetrahydro-pyran-2-yl)-1H-pyrazol-4-yl]-benzamide (10.14 g, 30.10 mmoles) in acetone (200 ml) was added MnO₂ (52.33 g, 602 mmoles). The resultant black suspension was stirred at ambient temperature for 48 hours. The reaction mixture was filtered through celite, and the fitrate evaporated in vacuo. The residue was purified by flash column chromatography (Biotage SP4, 40M, flow rate 30 ml/min, gradient 1:3 EtOAc/Petrol to 3:2 EtOAc/Petrol) to give 2,6-difluoro-N-[3-formyl-1-(tetrahydro-pyran-2-yl)-1H-pyrazol-4-yl]-benzamide as a creamy solid (7.8 g, 77%). (LC/MS: Rₜ 3.03, [M+H]⁺336.03).

### 4G. Synthesis of 2,6-difluoro-N-[3-(1-hydroxy-ethyl)-1-(tetrahydro-pyran-2-yl)-1H-pyrazol-4-yl]-benzamide

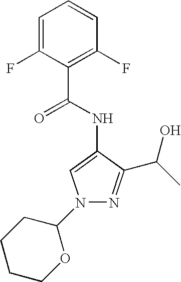

To a stirred solution of 2,6-difluoro-N-[3-formyl-1-(tetrahydro-pyran-2-yl)-1H-pyrazol-4-yl]-benzamide (2.0 g, 5.97 mmoles) in dry THF (20 ml) under a nitrogen atmosphere was added dropwise methyl magnesium bromide (1.4M in THF/Toluene) (26 ml, 40.6 mmoles). The resultant suspension was stirred at ambient temperature for 3 hours. Reaction mixture was quenched with saturated Ammonium Chloride then partitioned between EtOAc and 2N HCl. The organic portion was dried (MgSO₄), filtered and evaporated in vacuo. The residue was purified by flash chromatography (Biotage SP4, 40S,flow rate 40 ml/min, gradient 1:2 EtOAc/Petrol to 2:1 EtOAc/Petrol) to give 2,6-difluoro-N-[3-(1-hydroxy-ethyl)-1-(tetrahydro-pyran-2-yl)-1H-pyrazol-4-yl]-benzamide as a colourless oil (2.0 g, 95%). (LC/MS: Rₜ 2.48, [M+H]⁺352.27.

### 4H. Synthesis of N-[3-Acetyl-1-(tetrahydro-pyran-2-yl)-1H-Pyrazol-4-yl-2,6-difluoro-benzamide

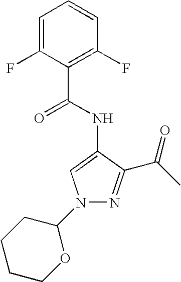

2,6-difluoro-N-[3-(1-hydroxy-ethyl)-1-(tetrahydro-pyran-2-yl)-1H-pyrazol-4-yl-benzamide (2 g, 5.70 mmol) dissolved in dichloromethane (30 ml), under a nitrogen atmosphere was added Dess-Martin Periodinane (2.7 g, 6.27 mmol) and the suspension stirred at room temperature for 20 hours. Reaction mixture evaporated in vacuo and crude purified by flash chromatography (Biotage SP4, 40M, flow rate 40 ml/min, gradient 1:5 EtOAc/Petrol to 2:1 EtOAc/Petrol) to give N-[3-Acetyl-1-(tetrahydro-pyran-2-yl)-1H-Pyrazol-4-yl-2,6-difluoro-benzamide as a white solid (1.2 g, 60%). (LC/MS: Rₜ 3.16, [M+H]⁺350.25).

### 4I. Synthesis of N-[3-(2-Bromo-acetyl)-1H-pyrazol-4-yl]-2,6-difluoro-benzamide

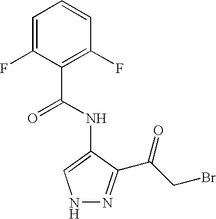

N-[3-Acetyl-1-(tetrahydro-pyran-2-yl)-1H-Pyrazol-4-yl]-2,6-difluoro-benzamide (1.2 g, 3.40 mmol) was dissolved in chloroform (20 ml), added acetic acid (8 ml) followed by slow addition of Bromine (383 μl, 7.48 mmol). Left reaction mixture stirred at 60° C. for a period of 25 hours. Residue was evaporated in vacuo, azeotroped with toluene and crude material was purified by flash chromatography [silica, EtOAc/Petrol (1:1)] to give N-[3-(2-Bromo-acetyl)-1H-pyrazol-4-yl-2,6-difluoro-benzamide (1 g, 30% yield) as a white solid. (LC/MS: Rₜ 2.83, [M+H]⁺344, 346).

### 4J. Synthesis of 2,6-Difluoro-N-[3-(2-methyl-thiazol-4-yl)-1H-pyrazol-4-yl]-benzamide

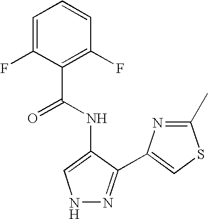

N-[3-(2-Bromo-acetyl)-1H-pyrazol-4-yl-2,6-difluoro-benzamide (0.047 g, 0.12 mmol) was dissolved in THF (2 ml), thioacetamide (0.01 g, 0.132 mmol) added to the reaction mixture and the suspension heated at 130° C. (150W) for 20 min in a CEM Discover™ microwave synthesiser. The reaction mixture was evaporated in vacuo and then azeotroped with toluene (2×5 ml).Diethyl ether (5 ml) was added to the crude material to give 2,6-Difluoro-N-[3-(2-methyl-thiazol-4-yl)-1H-pyrazol-4-yl]-benzamide 20 mg, 47% yield) as a white solid. (LC/MS: Rₜ 2.86, [M+H]⁺353, 355).

### Example 5

### Synthesis of 2,6-Difluoro-N-[3-(2-methyl-oxazol-4-yl)-1H-pyrazol-4-yl]-benzamide

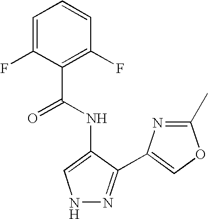

N-[3-(2-Bromo-acetyl)-1H-pyrazol-4-yl]-2,6-difluoro-benzamide (Example 4I, 0.06 g, 0.17 mmol) was dissolved in acetic acid (3 ml), ammonium acetate (0.054 g, 0.68 mmol) added to the reaction mixture and the suspension heated at 130° C. (150 W) for 30 min in a CEM Discover™ microwave synthesiser. To crude mixture was added K₂CO₃ (0.047 g.0.34 mmol) and refluxed (130° C.) for 2 hours. Evaporated in vacuo and then azeotroped with toluene (2×5 ml). Crude material was purified by preparative LC/MS to afford 2,6-Difluoro-N-[3-(2-methyl-oxazol-4-yl)-1H-pyrazol-4-yl]-benzamide (3 mg, 6% yield) as a pale yellow solid. (LC/MS: Rₜ 2.71, [M+H]⁺305).

### Example 6

### Synthesis of 2,6-Difluoro-N-[3-(2-trifluoromethyl-1H-imidazol-4-yl)-1H-pyrazol-4-yl]-benzamide

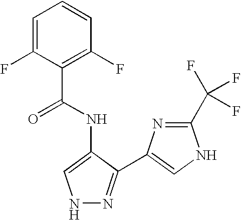

N-[3-(2-Bromo-acetyl)-1H-pyrazol-4-yl-2,6-difluoro-benzamide (Example 4I, 0.06 g, 0.17 mmol), Trifluoroacetamidine (0.025 g, 0.22 mmol), were placed in a microwave tube and heated at 150° C. (150W) for 30 min in a CEM Discover™ microwave synthesiser. Crude mixture was purified by flash column chromatography [SiO₂, Hexane:EtOAc, 1:2]. Further purification was required by preparative LCMS to give 2,6-Difluoro-N-[3-(2-trifluoromethyl-1H-imidazol-4-yl)-1H-pyrazol-4-yl-benzamide (10 mg, 16% yield) as a white solid. (LC/MS: Rₜ 2.83, [M+H]⁺358).

### Example 7

### Synthesis of 2,6-Difluoro-N-[3-(2-phenyl-thiazol-4-yl)-1H-pyrazol-4-yl]-benzamide

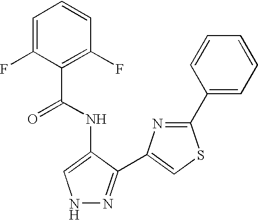

N-[3-(2-Bromo-acetyl)-1H-pyrazol-4-yl]-2,6-difluoro-benzamide (Example 4I, 0.06 g, 0.17 mmol) was dissolved in THF (2 ml), thiobenzamide (0.028 g, 0.2 mmol) added to the reaction mixture and the suspension heated at 130° C. (150W) for 30 min in a CEM Discover™ microwave synthesiser. The reaction mixture was evaporated in vacuo and then triturated with EtOAc and diethyl ether. Filtrate solid from solvents. Further purification was required by preparative LCMS to give 2,6-Difluoro-N-[3-(2-phenyl-thiazol-4-yl)-1H-pyrazol-4-yl]-benzamide as a white solid (20 mg, 31% yield) . (LC/MS: Rₜ 3.40, [M+H]⁺383).

### Example 8

### Synthesis of 2,6-Difluoro-N-(3-imidazo[1,2-a]pyridin-2-yl-1H-pyrazol-4-yl]-benzamide

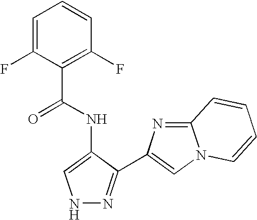

N-[3-(2-Bromo-acetyl)-1H-pyrazol-4-yl]-2,6-difluoro-benzamide (Example 4I, 0.068 g, 0.19 mmol), was dissolved in THF (2 ml), 2-amino-pyridine (0.019 g, 0.19 mmol) added to the reaction mixture and the suspension heated at 130° C. (150 W) for 20 min in a CEM Discover™ microwave synthesiser. The reaction mixture was evaporated in vacuo and then purified by flash column chromatography [SiO₂, Hexane:EtOAc, 1:1] to afford 2,6-Difluoro-N-(3-imidazo[1,2-a]pyridin-2-yl-1H-pyrazol-4-yl]-benzamide (15 mg, 25% yield) as a white solid. (LC/MS: Rₜ 1.87, [M+H]⁺340).

### Example 9

### Synthesis of 4-(2,6-dichlorobenzoylamino-3-(1-pyrazolyl)-1H-pyrazole

### 9A. 4-nitro-3-(1-pyrazolyl)-pyrazole

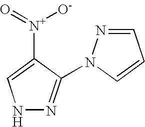

Concentrated nitric acid (3.5 ml) followed by acetic anhydride (5.5 ml) were added to a suspension of 4-nitropyrazole (3 g) in 18 ml of acetic acid, stirred at room temperature for 1 hr then poured onto ice. This was neutralised with solid sodium carbonate then extracted with ethyl acetate (2×100 ml). The combined organics were dried (MgSO₄) and evaporated to give 3.95 g of 1,4-dintropyrazole as a colourless oil.

A solution of pyrazole (430 mg; 6.3 mmol) in ethanol (10 ml) was added cautiously to 1,4-dinitropyrazole (500 mg; 3.15 mmol) in ethanol (10 ml) then heated at reflux for 3 hours. A solution of sulfamic acid (305 mg; 3.15 mmol) in 10 ml of water was added, then the reaction was allowed to cool, evaporated and the residue triturated with water (20 ml). Suction filtration afforded 340 mg of 4-nitro-3-(1-pyrazolyl)-pyrazole as a white solid. (LC/MS: Rₜ 1.82, [M+H]⁺180).

### 9B. 4-amino-3-(1-pyrazolyl)-pyrazole

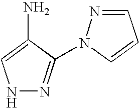

4-Nitro-3-(1-pyrazolyl)-pyrazole (320 mg; 1.78 mmol) was dissolved in ethanol (10 ml). 10% Palladium on carbon (50 mg) was added under a flow of nitrogen and the reaction mixture was shaken for 24 h under a hydrogen atmosphere. The reaction mixture was filtered through Celite, washed with ethanol and the filtrate reduced in vacuo to give 4-amino-3-(1-pyrazolyl)-pyrazole as a red/brown solid (265 mg). (LC/MS: Rₜ 0.33, [M+H]⁺150).

### 9C. 4-(2,6-dichlorobenzoylamino)-3-(1-pyrazolyl)-1H-pyrazole

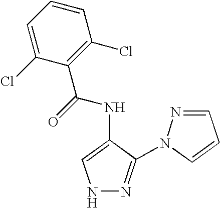

2,6-dichlorobenzoyl chloride (155 mg; 0.74 mmol) was added to a solution of 4-amino-3-(1-pyrazolyl)-pyrazole (100 mg; 0.67 mmol) and triethylamine (115 μl, 0.8 mmol) in 5 ml of dioxan then stirred at room temperature overnight. The reaction mixture was diluted with 10 ml of saturated NaHCO₃ solution, the solid was collected by filtration, washed with water and dried under vacuum to give the title compound as an off-white solid. (LC/MS: Rₜ 2.79, [M+H]⁺322).

### Example 10

### Synthesis of 4-(2,6-dichlorobenzoylamino-3-[1-(3-trifluoromethyl)-pyrazolyl]-1H-pyrazole

### 10A. 1-(4-methoxybenzyl)-3,4-dinitro-1H-pyrazole

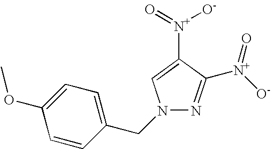

Potassium carbonate (2.1 g) was added to a stirred solution of 3,4-dinitropyrazole (2 g; 12.65 mmol) and 4-methoxybenzyl chloride (1.9 ml) in acetonitrile, the reaction was then heated at 55° C. overnight, cooled and evaporated. The residue was partitioned between EtOAc and brine. The organic layer was separated, dried (MgSO₄) and evaporated in vacuo. The residue was purified by flash chromatography (Biotage SP4, gradient elution 1:4 to 1:2 EtOAc/Petrol) to give 2.8 g of 1-(4-methoxy-benzyl)-3,4-dinitro-1H-pyrazole as a colourless gum. (LC/MS: Rₜ 3.13, [M+H]⁺ none).

### 10B. [1-(4-methoxy-benzyl)-4-nitro-1H-pyrazol-3-yl]-hydrazine

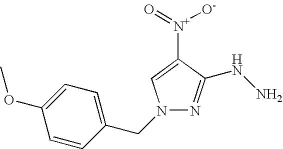

Hydrazine hydrate (2.4 ml) was added to a solution of 1-(4-methoxy-benzyl)-3,4-dinitro-1H-pyrazole (2.4 g) in 50 ml of isopropanol then heated at reflux overnight. The reaction was cooled to room temperature and the yellow solid collected by filtration, washed with isopropanol and sucked dry to give 1.75 g of product. (LC/MS: Rₜ 1.90, [M+H]⁺264).

### 10C. 1-(4-methoxybenzyl)-4-nitro-3-[1-(3-trifluoromethyl)-pyrazolyl]pyrazole

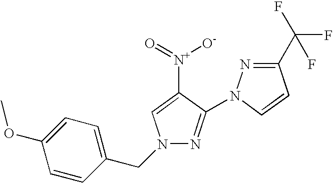

Concentrated hydrochloric acid (100 ul) was added to a suspension of [1-(4-methoxy-benzyl)-4-nitro-1H-pyrazol-3-yl]-hydrazine (570 mg) in 10 ml of ethanol, stirred for 2 minutes then added 4-ethoxy-1,1,1-trifluoro-but-3-en-2-one. The reaction was heated at 130° C. (100 W) for 10 min in a CEM Discover™ microwave synthesiser. The reaction mixture was evaporated in vacuo then partitioned between EtOAc and 2M HCl. The organic layer was separated, dried (MgSO4) and evaporated in vacuo. The residue was purified by flash chromatography (Biotage SP4, gradient elution 1:4 to 1:2 EtOAc/ Petrol) to give 145 mg of product and 540 mg of 1-(4-methoxybenzyl)-4-nitro-3-[1-(5-trifluoromethyl)-pyrazolyl)pyrazole. (LC/MS: Rₜ 3.36, [M+H]⁺368).

### 10D. 1-(4-methoxybenzyl)-4-amino-3-[1-(3-trifluoromethyl)-pyrazolyl]pyrazole

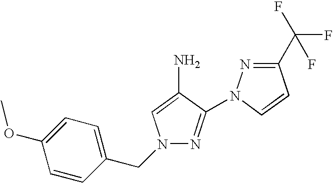

1-(4-methoxybenzyl)-4-nitro-3-[1-(3-trifluoromethyl)-pyrazolyl]pyrazole (190 mg) was dissolved in ethanol (10 ml). 10% Palladium on carbon (20 mg) was added under a flow of nitrogen then the reaction mixture was shaken for 24 h under a hydrogen atmosphere. The reaction mixture was filtered through Celite, washed with ethanol and the filtrate reduced in vacuo. The residue was purified by flash chromatography (Biotage SP4, 1:4 then 1:3 then 1:2 EtOAc/Petrol) to give 1-(4-methoxybenzyl)-4-amino-3-[1-(3-trifluoromethyl)-pyrazolyl)pyrazole as a pale yellow solid (95 mg). (LC/MS: Rₜ 3.20, [M+H]⁺338).

### 10E. 4-(2,6-dichlorobenzoylamino)-3-[1-(3-trifluoromethyl)-pyrazolyl]pyrazole

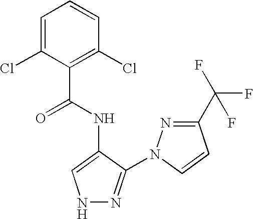

2,6-dichlorobenzoyl chloride (40 μl) was added to a solution of 1-(4-methoxybenzyl)-4-amino-3-[1-(3-trifluoromethyl)-pyrazolyl) pyrazole (95 mg; 0.28 mmol) and triethylamine (50 μl) in 5 ml of dioxan then stirred at room temperature overnight. The reaction mixture was evaporated and the residue partitioned between saturated NaHCO₃ solution and DCM. The organic layer was separated, dried (MgSO₄) and evaporated in vacuo. The residue was purified by flash chromatography (Biotage SP4, 1:4 to 1:2 EtOAc/ Petrol) to give 70 mg of the amide intermediate. This was treated with trifluoroacetic acid (1 ml) and anisole (70 μl) then heated at 120° C. for 10 min in a CEM Discover™ microwave synthesiser. The reaction mixture was evaporated in vacuo then re-evaporated with toluene (×2). Purification by flash chromatography (Biotage SP4, 1:4 to 1:2 EtOAc/Petrol) gave 40 mg of 4-(2,6-dichlorobenzoylamino)-3-[1-(3-trifluoromethyl)-pyrazolyl]pyrazole as an off white solid. (LC/MS: Rₜ 3.14, [M+H]⁺390).

### Example 11

### Synthesis of N-(1H,1′H-[3,4′]Bipyrazolyl-4-yl)-2,6-difluoro-benzamide

### 11 A. 4-Nitro-1H,1′H-[3,4]bipyrazolyl

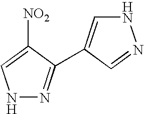

3-(Dimethylamino)-2-{4-nitro-1H-pyrazol-3-yl}acrylaldehyde (SPECS AG-205/13631581) (250 mg, 1.2 mmol), and the hydrazine (64 μl, 1.3 mmol) was heated to 80° C. in EtOH (2 ml) and water (5 ml) over night. The reaction was cooled and evaporated to dryness and purified by flash chromatography to yield the product. (LC/MS Acidic: RT 1.79, [M+H]⁺180).

### 11B. 1H,1′H-[3,4′Bipyrazolyl-4-ylamine

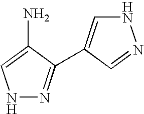

The nitro compound 2 was taken up in EtOH and Pd/C (10%) added under N₂. The atmosphere was exchanged for H₂ and the reaction was left at room temperature over night. The reaction was filtered and evaporated to dryness to yield the product. (LC/MS Acidic: RT 0.36, [M+H]⁺150).

### 11 C. N-(1H,1′H-[3,4]Bipyrazolyl-4-yl)-2,6-difluoro-benzamide

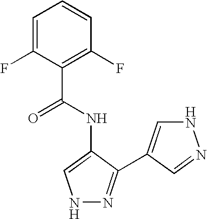

To 1H,1′H-[3,4′]Bipyrazolyl-4-ylamine (1.2 mmol) was added EDC (223 mg, 1.2 mmol), HOAt (200 mg, 1.2 mmol), 2,6,-difluorobenzoic acid (228 mg, 1.2 mol) and DCM:DMF (1:1, 20 ml). The reaction was stirred under N₂ over night. The reaction was worked up by pouring into water and extracting with EtOAc (×2). The combined organic layers were washed with brine and dried over MgSO₄. The product was filtered and evaporated to dryness. The products were taken up in dioxane and 1N NaOH (4:1) was added. The reaction was stirred at room temperature for 6 hours, and then neutralised with 1M HCl. The product was extracted with EtOAc (×2). The combined organic layers were washed with brine and dried over MgSO₄. The product was filtered and evaporated to dryness. Purification was flash chromatography to yield the product. (LC/MS Acidic: RT 1.88, [M+H]⁺290).

### Example 12

### Synthesis of 2,6-dichloro-N-(3-oxazol-5-yl-1H-pyrazol-4-yl)-benzamide

### 12A. Synthesis of 4-Nitro-1H-pyrazole-3-carboxylic acid methyl ester

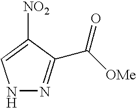

Thionyl chloride (3.8 ml, 52.5 mmol) was added cautiously to a stirred, ice-cold mixture of 4-nitropyrazole-3-carboxylic acid (7.5 g, 47.7 mmol) in MeOH (150 ml), the mixture stirred at ambient temperature for 1 hour, then heated at reflux for 3 hours. The reaction mixture was cooled, evaporated in vacuo and then azeotroped with toluene to give 4-nitro-1H-pyrazole-3-carboxylic acid ethyl ester (8.8 g).

### 12B. Synthesis of 4-Nitro-1-(tetrahydro-pyran-2-yl)-1H-pyrazole-3-carboxylic acid methyl ester

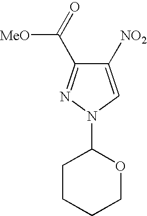

A suspension of 4-nitro-1H-pyrazole-3-carboxylic acid methyl ester (5 g, 29.24 mmol) and p-toluene sulphonic acid (555 mg, 2.92 mmol) in chloroform (100 ml) at 0° C. was treated with 3,4-dihydropyran (4 ml, 43.8 mmol) dropwise. The reaction mixture was allowed to warm to ambient temperature, and then stirred for a further 2 hours. The reaction mixture was diluted with Et₂O, washed sequentially with saturated NaHCO₃ solution and brine. The organic portion was dried (MgSO₄), filtered and evaporated in vacuo. The residue was purified by flash chromatography [silica, EtOAc/Petrol (1:2)] to give 4-nitro-1-(tetrahydro-pyran-2-yl)-1H-pyrazole-3-carboxylic acid methyl ester (7.1 g, 95%) as a colourless oil. (LC/MS: Rₜ 2.86, [M+H]⁺256.00).

### 12C. Synthesis of 4-amino-1-(tetrahydro-pyran-2-yl)-1H-pyrazole-3-carboxylic acid methyl ester

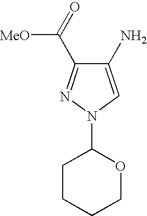

To a stirred solution of 4-nitro-1-(tetrahydro-pyran-2-yl)-1H-pyrazole-3-carboxylic acid methyl ester (16.0 g, 62.75 mmoles) and ammonium formate (39.6 g, 627.45 mmoles) in ethanol (200 ml) and water (20 ml) under nitrogen was added palladium on carbon (10%, 0.8 g). The reaction mixture was heated at 50° C. for 2 hours. The suspension was filtered through celite, and the filtrate was partitioned between EtOAc and water. The organic portion was dried (MgSO₄), filtered and evaporated in vacuo to give 4-amino-1-(tetrahydro-pyran-2-yl)-1H-pyrazole-3-carboxylic acid methyl ester as a yellow oil (12.5 g, 89%). (LC/MS: Rₜ 1.84, [M+H]⁺226.06).

### 12D. Synthesis of 4-(2,6-dichloro-benzoylamino)-1-(tetrahydro-pyran-2-yl)-1H-pyrazole-3-carboxylic acid methyl ester

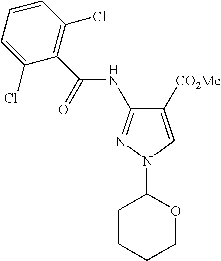

A solution of 4-amino-1-(tetrahydro-pyran-2-yl)-1H-pyrazole-3-carboxylic acid methyl ester (6.0 g, 26.67 mmoles), triethylamine (4.4 ml, 32.00 mmoles) and 2,6-dichlorobenzoyl chloride (4.2 ml, 29.34 mmoles) in dichloromethane (100 ml) was stirred at ambient temperature for 2 hours. Further triethylamine (1.1 ml, 7.91 mmoles) and 2,6-dichlorobenzoyl chloride (1 ml, 6.98 mmoles) were added to the reaction mixture and the resultant solution stirred at ambient temperature for a further 3 hours. The reaction mixture was washed with water, dried (MgSO₄), filtered and evaporated in vacuo. The residue was purified by flash chromatography (Biotage SP4, 40M,flow rate 40 ml/min, gradient 1:2 EtOAc/Petrol to 4:1 EtOAc/Petrol) to give 4-(2,6-dichloro-benzoylamino)-1-(tetrahydro-pyran-2-yl)-1H-pyrazole-3-carboxylic acid methyl ester as a white solid (8.0 g, 75%). (LC/MS: Rₜ 3.16, [M+H]⁺398.04.

### 12E. Synthesis of 2,6-dichloro-N-[3-hydroxymethyl-1-(tetrahydro-pyran-2-yl)-1H-pyrazol-4-yl]-benzamide

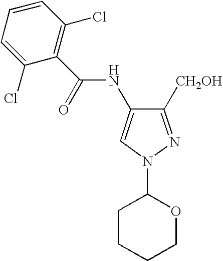

A stirred solution of 4-(2,6-dichloro-benzoylamino)-1-(tetrahydro-pyran-2-yl)-1H-pyrazole-3-carboxylic acid methyl ester (8.0 g, 20.1 mmoles) in THF (80 ml) under nitrogen at −78° C. was treated dropwise with a solution of diisobutylaluminium hydride in THF (160 ml, 1M). The reaction mixture was stirred at −78° C. for 30 minutes and then warmed to 0° C. in an ice-water bath. A saturated aqueous solution of sodium sulphate was added to the reaction mixture. The suspension was filtered through celite. The filtrate was partitioned between EtOAc and brine. The organic portion was dried (MgSO₄), filtered and evaporated in vacuo to give 2,6-dichloro-N-[3-hydroxymethyl-1-(tetrahydro-pyran-2-yl)-1H-pyrazol-4-yl]-benzamide as a white solid (4.0 g, 53%). (LC/MS: Rₜ 2.50, [M+H]⁺370.06).

### 12F. Synthesis of 2,6-dichloro-N-[3-formyl-1-(tetrahydro-pyran-2-yl)-1H-pyrazol-4-yl]-benzamide

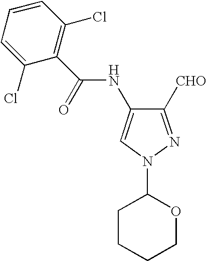

To a stirred solution of 2,6-dichloro-N-[3-hydroxymethyl-1-(tetrahydro-pyran-2-yl)-1H-pyrazol-4-yl]-benzamide (4.0 g, 10.81 mmoles) in acetone (40 ml) was added MnO₂ (18.8 g, 216.22 mmoles). The resultant black suspension was stirred at ambient temperature for 24 hours. The reaction mixture was filtered through celite, and the filtrate evaporated in vacuo to give 2,6-dichloro-N-[3-formyl-1-(tetrahydro-pyran-2-yl)-1H-pyrazol-4-yl]-benzamide as a white solid (3.3 g, 83%). (LC/MS: Rₜ 3.22, [M+H]⁺368.02).

### 12G. Synthesis of 2,6-dichloro-N-[3-oxazol-5-yl-1-(tetrandro-pyran-2-yl)-1H-pyrazol-4-yl]-benzamide

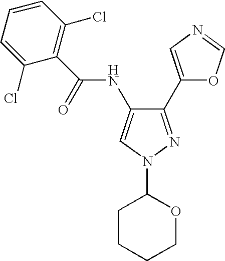

To a stirred solution of 2,6-dichloro-N-[3-formyl-1-(tetrahydro-pyran-2-yl)-1H-pyrazol-4-yl]-benzamide (400 mg, 1.09 mmoles) in methanol was added p-toluenesulphonylmethyl isocyanide (213 mg, 1.09 mmoles) and potassium carbonate (150 mg, 1.09 mmoles). The reaction mixture was heated at reflux for 1 hour. Further potassium carbonate (150 mg, 1.09 mmoles) was added to the reaction mixture and the resultant solution heated at reflux for a further 2 hours. The reaction mixture was diluted with EtOAc and washed with water and then brine. The organic portion was dried (MgSO₄), filtered and evaporated in vacuo. The residue was purified by flash chromatography (Biotage SP4, 25S, flow rate 25 ml/min, gradient 3:7 EtOAc/Petrol to 7:3 EtOAc/Petrol) to give 2,6-dichloro-N-[3-oxazol-5-yl-1-(tetrandro-pyran-2-yl)-1H-pyrazol-4-yl]-benzamide as a yellow solid (210 mg, 47%). (LC/MS: Rₜ 2.78, [M+H]⁺407.19).

12H. Synthesis of 2,6-dichloro-N-(3-oxazol-5-yl-1H-pyrazol-4-yl)-benzamide

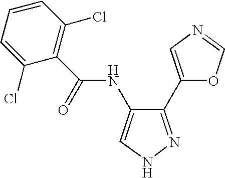

To a solution of 2,6-dichloro-N-[3-oxazol-5-yl-1-(tetrandro-pyran-2-yl)-1H-pyrazol-4-yl]-benzamide (210 mg, 0.516 mmoles) in ethanol (10 ml) was added toluenesulphonic acid (297 mg, 1.548 mmoles) and the resultant solution heated at 70° C. for 1 hour. The ethanol was evaporated in vacuo. The residue was partitioned between EtOAc and a saturated aqueous solution of sodium hydrogen carbonate. The organic portion was dried (MgSO₄), filtered and evaporated in vacuo. The residue was purified by flash chromatography (Biotage SP4, 25S, flow rate 25 ml/min, gradient 1:1 EtOAc/ Petrol to EtOAc) to give 2,6-dichloro-N-(3-oxazol-5-yl-1H-pyrazol-4-yl)-benzamide as an off-white solid (15 mg, 10%). (LC/MS: Rₜ 2.16, [M+H]⁺323.11).

### Examples 13-20

| Example | | | |
|---|---|---|---|
| No. | Structure | Method of Preparation | LCMS |
| 13 | 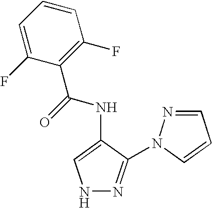 | As for Example 9, but using 2,6-difluorobenzoyl chloride in 9C. | [M + H]⁺ 290 Rₜ 2.62 |
| 14 | 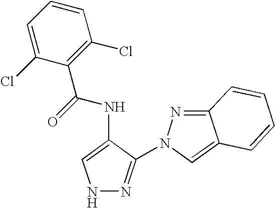 | As for Example 9, but using indazole in 9A. | [M + H]⁺ 372 Rₜ 3.14 |
| 15 | 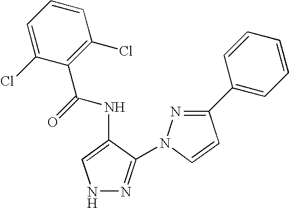 | As for Example 9, but using 3-phenylpyrazole in 9A. | [M + H]⁺ 398 Rₜ 3.59 |
| 16 | 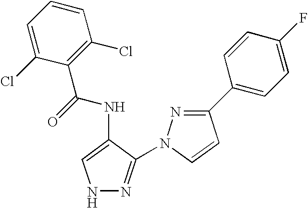 | As for Example 9, but using 3-(4-fluorophenyl)pyrazole in 9A. | [M + H]⁺ 416 Rₜ 3.56 |
| 17 | 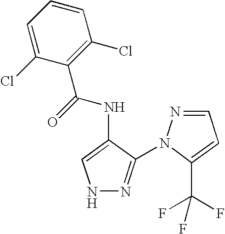 | As for Example 10 - 5- trifluoromethyl isomer obtained as a by-product during the cyclisation step. | [M + H]⁺ 390 Rₜ 2.83 |
| 19 | 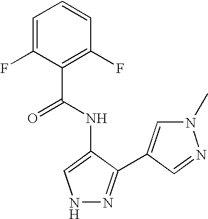 | As for Example 11 but using methyl hydrazine in 11A. | [M + H]⁺ 304 Rₜ 1.99 |
| 20 | 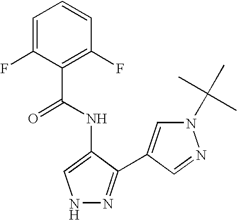 | As for Example 11 but using t-butyl hydrazine in 11A. | [M + H]⁺ 346 Rₜ 2.45 |

### Example 21

Synthesis of 5-Chloro-N-[3-(1-isopropyl-5-oxo-4,5-dihydro-1H-[1,2,4]triazol-3-yl)-1H-pyrazol-4-yl]-2-methoxy-benzamide

### 21A. Synthesis of 4-Nitro-1-(tetrahydro-pyran-2-yl)-1H-pyrazole-3-carboxylic acid hydrazide

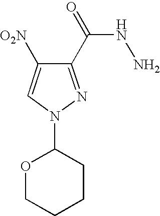

3,4-dihydropyran (8.21 ml, 90 mmol) was added drop wise to a solution of 4-nitro-1H-pyrazole-3-carboxylic acid methyl ester (10 g, 60 mmol) in chloroform (200 ml) at 0° C. The reaction mixture was stirred for 45 min before being allowed to warm to 25° C. After 1 h Et₂O (100 ml) was added and this mixture was washed sequentially with sat. aq. NaHCO₃ (250 ml), water (2×250 ml), and brine (250 ml). The organics were dried (Na₂SO₄) and evaporated in vacuo to give 4-nitro-1-(tetrahydro-pyran-2-yl)-1H-pyrazole-3-carboxylic acid methyl ester as a yellow oil (16.99 g, impure). A portion of this protected pyrazole (9.66 g, 37.88 mmol) was combined with hydrazine hydrate (9.97 ml, 200 mmol) and ethanol (150 ml) and stirred at 25° C. under N₂. After 1 h the mixture was evaporated in vacuo to give a crude orange oil. This was partitioned between EtOAc (4×150 ml) and brine (150 ml). The organic layer was dried (Na₂SO₄) and evaporated in vacuo to give 4-nitro-1-(tetrahydro-pyran-2-yl)-1H-pyrazole-3-carboxylic acid hydrazide as a yellow foam (8.45 g, 87%). (LC/MS (acidic method): Rₜ 1.56, [M+H]⁺256).

### 21B. Synthesis of 4-Nitro-1-(tetrahydro-pyran-2-yl)-1H-pyrazole-3-carboxylic acid N′-isopropyl-hydrazide

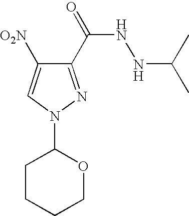

3A powdered sieves (1 g), acetone (0.43 ml, 5.88 mmol), NaCNBH₃ (493 mg, 7.84 mmol) and AcOH (1.12 ml, 19.6 mmol) were added sequentially to a solution of 4-nitro-1-(tetrahydro-pyran-2-yl)-1H-pyrazole-3-carboxylic acid hydrazide (1 g, 3.92 mmol) in MeOH (35 ml). After 2 h the reaction mixture was filtered, and the filtrate evaporated in vacuo. The resultant crude was partitioned between EtOAc (100 ml) and sat. aq. NaHCO₃ (100 ml). The dried (Na₂SO₄) organic layer was evaporated in vacuo to give a crude oil. Flash chromatography (SiO₂), eluting with DCM—3% MeOH/DCM afforded 4-nitro-1-(tetrahydro-pyran-2-yl)-1H-pyrazole-3-carboxylic acid N′-isopropyl-hydrazide as a yellow solid (1.05 g, 90%). (LC/MS (acidic method): Rₜ 2.30, [M+H]⁺298).

### 21C. Synthesis of 2-Isopropyl-4-(4-methoxy-benzyl)-5-[4-nitro-1-(tetrahydro-pyran-2-yl)-1H-pyrazol-3-yl-2,4-dihydro-[1,2,4]triazol-3-one

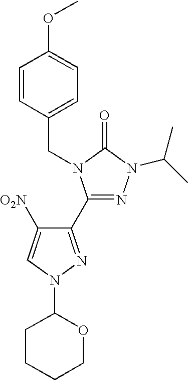

Paramethoxybenzyl isocyanate (0.55 ml, 3.88 mmol) was added dropwise to a solution of 4-nitro-1-(tetrahydro-pyran-2-yl)-1H-pyrazole-3-carboxylic acid N′-isopropyl-hydrazide (1.05 g, 3.53 mmol) at 0° C. After 10 min the reaction was allowed to warm to 25° C. and left to stir overnight. The reaction mixture was evaporated in vacuo to give a crude oil. Flash chromatography, eluting with CH₂Cl₂-3% MeOH/CH₂Cl₂ afforded the intermediate hydrazide urea as a yellow oil. This was dissolved in 2N aq. NaOH (50 ml) and heated at 100° C. for 1 h. The reaction was allowed to cool to RT before being extracted with EtOAc (3×50 ml). The combined, dried (Na₂SO₄) organics were evaporated in vacuo to give a crude solid. Flash chromatography, eluting with 20% EtOAc/Petrol—45% EtOAc/Petrol afforded 2-Isopropyl-4-(4-methoxy-benzyl)-5-[4-nitro-1-(tetrahydro-pyran-2-yl)-1H-pyrazol-3-yl-2,4-dihydro-[1,2,4]triazol-3-one as a white solid (1 g, 64%) (LC/MS (acidic method): Rₜ 3.1, [M+H]⁺443.25).

### 21D. Synthesis of 5-[4-Amino-1-(tetrahydro-pyran-2-yl)-1H-pyrazol-3-yl]-2-isopropyl-4-(4-methoxy-benzyl)-2,4-dihydro-[1,2,4]triazol-3-one

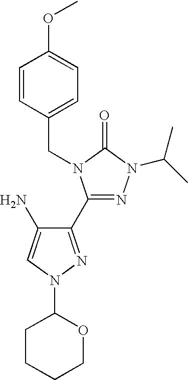

Palladium (10% on carbon, 25 mg) was added to a solution of 2-isopropyl-4-(4-methoxy-benzyl)-5-[4-nitro-1-(tetrahydro-pyran-2-yl)-1H-pyrazol-3-yl-2,4-dihydro-[1,2,4]triazol-3-one (500 mg, 1.13 mmol) in EtOH (20 ml). The mixture was agitated under H₂ for 6 h. The reaction mixture was then filtered through a pad of celite, and evaporated in vacuo. Flash chromatography (SiO₂), eluting with 30% EtOAc/Petrol—60% EtOAc/Petrol afforded 5-[4-amino-1-(tetrahydro-pyran-2-yl)-1H-pyrazol-3-yl]-2-isopropyl-4-(4-methoxy-benzyl)-2,4-dihydro-[1,2,4]triazol-3-one as a colourless oil (500 mg, 100%) (LC/MS (acidic method): Rₜ 2.76, [M+H]⁺413).

### 21E. Synthesis of 5-Chloro-N-[3-(1-isopropyl-5-oxo-4,5-dihydro-1H-[1,2,4]triazol-3-yl)-1H-pyrazol-4-yl]-2-methoxy-benzamide

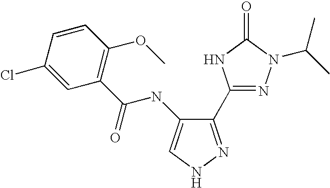

EDCI (238 mg, 1.24 mmol) was added to a mixture of 5-[4-amino-1-(tetrahydro-pyran-2-yl)-1H-pyrazol-3-yl-2-isopropyl-4-(4-methoxy-benzyl)-2,4-dihydro-[1,2,4]triazol-3-one (500 mg, 1.13 mmol), 5-chloro-2-methoxybenzoic acid (132 mg, 1.24 mmol) HOBT (168 mg, 1.24 mmol) and MeCN (20 ml). The reaction mixture was stirred at 25° C. for 2 h. The mixture was then evaporated in vacuo and partitioned between EtOAc (3×50 ml) and sat. aq. NaHCO₃ (50 ml). The combined, dried (Na₂SO₄) organics were evaporated in vacuo to give a crude oil. Flash chromatography (SiO₂), eluting with 20% EtOAc/Petrol—50% EtOAc/Petrol afforded the intermediate amide as a white foam (533 mg, 81%) (LC/MS (acidic method): Rₜ 3.83, [M+H]⁺581). This amide (312 mg, 0.537 mmol) was combined with TFA (3 ml) and anisole (0.24 ml, 2.15 mmol) and heated in a microwave reactor for 15 min. at 130° C. The reaction mixture was then evaporated in vacuo and triturated with Et₂O and CH₂Cl₂ to give 5-chloro-N-[3-(1-isopropyl-5-oxo-4,5-dihydro-1H-[1,2,4]triazol-3-yl)-1H-pyrazol-4-yl]-2-methoxy-benzamide as a pale coloured solid (150 mg, 74%). (LC/MS (acidic method/night): Rₜ 10.04, [M+H]⁺377).

### Examples 22-25

By using a method analogous the procedure described in Example 21 the compounds set out in the table below were prepared.

| EXAMPLE | | Differences to | |
|---|---|---|---|
| No. | Structure | General Procedure? | LCMS |
| 22 | 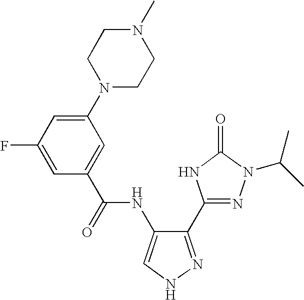 | Chromatograhy solvent sytem used: DCM- 10% MeOH/DCM. Heated in microwave for 1 h. Isolated as TFA salt. | [M + H]⁺ 429 Rₜ 6.15 (9-95 acidic/final) |
| 23 | 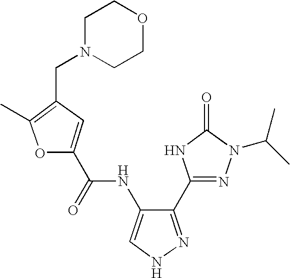 | Chromatograhy solvent sytem used: DCM- 10% MeOH/DCM. Heated in microwave for 1 h. Isolated as TFA salt. | [M + H]⁺ 416 Rₜ 5.58 (9-95 acidic/final) |
| 24 | 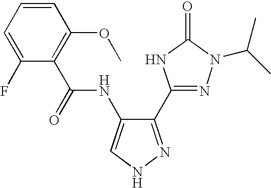 | Chromatograhy solvent sytem used: DCM- 10% MeOH/DCM. Heated in microwave for 1 h. | [M + H]⁺ 361 Rₜ 8.72 (9-95 acidic/final) |
| 25 | 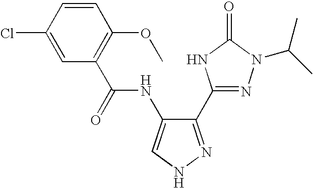 | | [M + H]⁺ 377 Rₜ 10.04 (9-95 acidic/final) |

### Example 26

### Synthesis of 5-Chloro-2-methoxy-N-{1-(4-methoxy-benzyl)-3-[4-(4-methoxy-benzyl)-1-methyl-5-oxo-4,5-dihydro-1H-[1,2,4]triazol-3-yl]-1H-pyrazol-4-yl}-benzamide

### 26A. Synthesis of 1-(4-methoxy-benzyl)-4-nitro-1H-pyrazole-3-carboxylic acid methyl ester

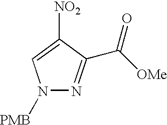

To a solution of 4-nitro-1H-pyrazole-3-carboxylic acid methyl ester (Example 4A, 8.8 g, 47.5 mmol) in MeCN (100 ml) was added K₂CO₃ (7.9 g, 57.0 mmol) followed by 4-methoxybenzyl chloride (7.1 ml, 52.3 mmol) and the mixture stirred at ambient temperature for 20 hours. The mixture was evaporated in vacuo, the residue partitioned between EtOAc and 2M aqueous hydrochloric acid and the organic portion washed with saturated aqueous sodium hydrogen carbonate, dried (MgSO₄) and evaporated in vacuo. The residue was purified by flash column chromatography [SiO₂, EtOAc-hexane (1:4)] to give 1-(4-methoxy-benzyl)-4-nitro-1H-pyrazole-3-carboxylic acid methyl ester (11 g) as a colourless gum.

### 26B. Synthesis of 1-(4-Methoxy-benzyl)-4-nitro-1H-pyrazole-3-carboxylic acid hydrazide

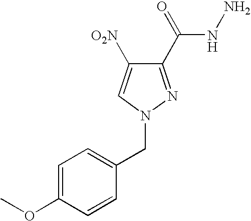

Hydrazine hydrate (9.46 ml, 199 mmol) was added to a mixture of 1-(4-methoxy-benzyl)-4-nitro-1H-pyrazole-3-carboxylic acid methyl ester (11 g, 37.8 mmol) in EtOH (150 ml). The reaction mixture was stirred at 25° C. for 2 h before being evaporated in vacuo and the resulting solid triturated with CH₂Cl₂ to give 1-(4-methoxy-benzyl)-4-nitro-1H-pyrazole-3-carboxylic acid hydrazide as a white solid (8.83 g, 80%) (LC/MS (acidic method): Rₜ 2.13, [M+H]⁺292).

### 26C. Synthesis of 4-(4-Methoxy-benzyl)-5-[1-(4-methoxy-benzyl)-4-nitro-1H-pyrazol-3-yl]-2,4-dihydro-[1,2,4]triazol-3-one

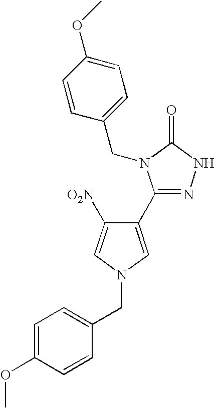

Paramethoixybenzylisocyanate (1.62 ml, 11.34 mmol) was added dropwise to an ice cooled solution of 1-(4-methoxy-benzyl)-4-nitro-1H-pyrazole-3-carboxylic acid hydrazide (3 g, 10.3 mmol) in THF (70 ml). After 10 min the reaction was allowed to warm to 25° C. Et₂O was then added added to the reaction mixture. The desired hydrazide urea intermediate was filtered off as a white solid (5 g, 100%) (LC/MS (acidic method): Rₜ 2.84, [M+H]⁺455). A portion of this material (3 g, 6.6 mmol) was added to 2N aq. NaOH (100 ml) and heated in a microwave reactor for 15 min at 140° C. The reaction mixture was partitioned between sat. aq. NH₄Cl solution (200 ml) and EtOAc (3×200 ml). The combined, dried (Na₂SO₄) organics were evaporated in vacuo to give a crude solid. This was then triturated with Et₂O to give 4-(4-methoxy-benzyl)-5-[1-(4-methoxy-benzyl)-4-nitro-1H-pyrazol-3-yl]-2,4-dihydro-[1,2,4]triazol-3-one as a pale coloured solid (1.29 g, 45%) (LC/MS (acidic method): Rₜ 2.82, [M+H]⁺437).

### 26D. Synthesis of 4-(4-Methoxy-benzyl)-5-[1-(4-methoxy-benzyl)-4-nitro-1H-pyrazol-3-yl]-2-methyl-2,4-dihydro-[1,2,4]triazol-3-one

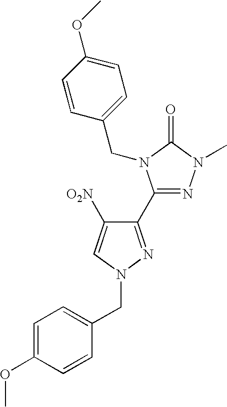

4-(4-Methoxy-benzyl)-5-[1-(4-methoxy-benzyl)-4-nitro-1H-pyrazol-3-yl]-2,4-dihydro-[1,2,4]triazol-3-one (0.1 g, 0.22 mmol) was suspended in EtOH (0.5 ml) and treated with 2M NaOH (0.125 ml, 0.25 mmol) followed by MeI (0.017 ml, 0.26 mmol). The reaction mixture was heated in a CEM discover microwave synthesizer (50 W) at 70° C. until the reaction was complete. On cooling a solid formed which was filtered and washed with EtOH to give 4-(4-methoxy-benzyl)-5-[1-(4-methoxy-benzyl)-4-nitro-1H-pyrazol-3-yl]-2-methyl-2,4-dihydro-[1,2,4]triazol-3-one (56 mg), (LC/MS Acidic: Rₜ 2.93, [M+H]⁺451).

### 26E. Synthesis of 5-[4-Amino-1-(4-methoxybenzyl)-1H-pyrazol-3-yl]-4-(4-methoxy-benzyl)-2-methyl-2,4-dihydro-[1,2,4]triazol-3-one

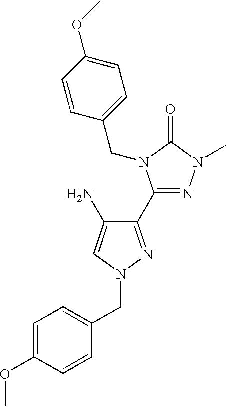

4-(4-Methoxy-benzyl)-5-[1-(4-methoxy-benzyl)-4-nitro-1H-pyrazol-3-yl]-2-methyl-2,4-dihydro-[1,2,4]triazol-3-one (0.248 g,0.5 mmol) was suspended in a mixture of EtOAc/MeOH. The suspension was shaken under a hydrogen atmosphere at ambient temperature for 6 h, then filtered through GF/A paper and reduced in vacuo to give 5-[4-Amino-1-(4-methoxybenzyl)-1H-pyrazol-3-yl]-4-(4-methoxy-benzyl)-2-methyl-2,4-dihydro-[1,2,4]triazol-3-one (0.18 g), (LC/MS Acidic: Rₜ 2.65, [M]⁺421).

### 26F. Synthesis of 5-Chloro-2-methoxy-N-{1-(4-methoxy-benzyl)-3-[4-(4-methoxy-benzyl)-1-methyl-5-oxo-4,5-dihydro-1H-[1,2,4]triazol-3-yl]-1H-pyrazol-4-yl}-benzamide

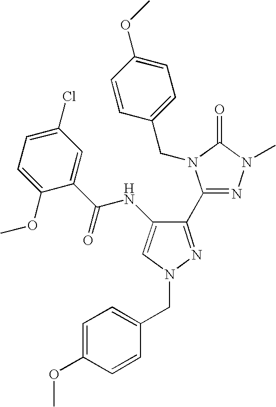

A mixture of 5-[4-Amino-1-(4-methoxybenzyl)-1H-pyrazol-3-yl-4-(4-methoxy-benzyl)-2-methyl-2,4-dihydro-[1,2,4]triazol-3-one (90 mg, 0.21 mmol), 5-chloro-2-methoxy-benzoic acid (0.044 g, 0.23 mmol), EDC (0.045 g, 0.23 mmol) and HOBt (0.032 g, 0.23 mmol) in CH₃CN (5 ml) was stirred at ambient temperature for 16 h.

The reaction was worked up by pouring into sat. NaHCO₃ and extracting with EtOAc, the organic layer was washed with water, dried (MgSO₄) and reduced in vacuo to give 5-chloro-2-methoxy-N-{1-(4-methoxy-benzyl)-3-[4-(4-methoxy-benzyl)-1-methyl-5-oxo-4,5-dihydro-1H-[1,2,4]triazol-3-yl]-1H-pyrazol-4-yl}-benzamide (0.124 g), (LC/MS Acidic: Rₜ 3.74, [M+H]⁺589).

### 26G. Synthesis of 5-Chloro-2-methoxy-N-[3-(1-methyl-5-oxo-4,5-dihydro-1H-[1,2,4]triazol-3-yl)-1H-pyrazol-4-yl]-benzamide

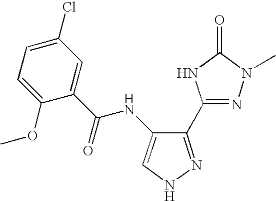

5-Chloro-2-methoxy-N-{1-(4-methoxy-benzyl)-3-[4-(4-methoxy-benzyl)-1-methyl-5-oxo-4,5-dihydro-1H-[1,2,4]triazol-3-yl]-1H-pyrazol-4-yl}-benzamide (0.124 g, 0.043 mmol) was dissolved in TFA (1.5 ml) then treated with anisole (0.091 ml) and heated in a CEM discover microwave synthesizer (50 W) at 130° C. until the reaction was complete. The reaction was reduced in vacuo and triturated with DMSO then washed with MeOH, CH₂Cl₂ followed by ether to give 5-chloro-2-methoxy-N-[3-(1-methyl-5-oxo-4,5-dihydro-1H-[1,2,4]triazol-3-yl)-1H-pyrazol-4-yl-benzamide (0.04 g), (LC/MS Acidic: Rₜ 2.59, [M+H]⁺349).

### Example 27

### Synthesis of 2,6-Difluoro-N-[3-(4-methyl-5-thioxo-4,5-dihydro-1H-[1,2,4]triazol-3-yl)-1H-pyrazol-4-yl]-benzamide

### 27A. Synthesis of 4-(2,6-Difluoro-benzoylamino)-1H-pyrazole-3-carboxylic acid methyl ester

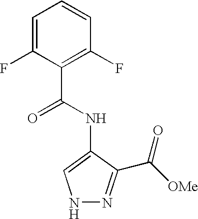

4-nitro-1H-pyrazole-3-carboxylic acid was converted to its methyl ester using thionyl chloride and methanol in a method analogous to that used in Example 3A. The nitro group was hydrogenated to the amine and this was then coupled to 2,6-difluorobenzoic acid using EDCI coupling conditions to provide 4-(2,6-Difluoro-benzoylamino)-1H-pyrazole-3-carboxylic acid methyl ester as outlined in Example 3C.

### 27B. Synthesis of 2,6-Difluoro-N-(3-hydrazinocarbonyl-1H-pyrazol-4-yl)-benzamide

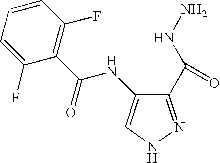

4-(2,6-Difluoro-benzoylamino)-1H-pyrazole-3-carboxylic acid methyl ester (0.3 g, 1.06 mmol) was suspended in ethanol (4 ml), then treated with hydrazine hydrate (0.258 ml, 5.3 mmol) and heated at 80° C. for 2 hours. The reaction was allowed to cool to RT and the solid that formed was filtered and dried to give 2,6-difluoro-N-(3-hydrazinocarbonyl-1H-pyrazol-4-yl)-benzamide (0.18 g) (LC/MSAcidic: Rₜ 1.85, [M+H]⁺281).

### 27C. Synthesis of 2,6-Difluoro-N-[3-(4-methyl-5-thioxo-4,5-dihydro-1H-[1,2,4]triazol-3-yl)-1H-pyrazol-4-yl-benzamide

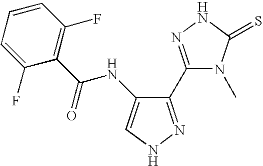

2,6-Difluoro-N-(3-hydrazinocarbonyl-1H-pyrazol-4-yl)-benzamide (0.05 g, 0.1 7 mmol), methyl isothiocyanate (15.6 mg, 0.21 mmol) in 1-butanol (1.5 ml) was heated at 120° C. for 10 min, DBU (0.015 ml) was added and reaction heated for a further 1.5 h. The reaction was reduced in vacuo and then purified through preparative LC/MS to give 2,6-difluoro-N-[3-(4-methyl-5-thioxo-4,5-dihydro-1H-[1,2,4]triazol-3-yl)-1H-pyrazol-4-yl-benzamide (18 mgs) (LC/MS Acidic: Rₜ 2.27, [M+H]⁺337).

### Example 28

### Synthesis of 5-Methanesulfonyl-2-methoxy-N-(3-pyridin-2-yl-1H-pyrazol-4-yl)benzamide

### 28A. Synthesis of 2-(4-Nitro-1H-pyrazol-3-yl)-pyridine

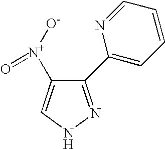

2-(1H-pyrazole-3-yl) pyridine (Maybridge) (0.63 g, 4.34 mmol) was dissolved in c. H₂SO₄ (10 ml) and treated with a mixture of c. H₂SO₄ (5 ml) and c. HNO₃ (5 ml). This mixture was stirred at ambient temperature O/N, then heated at 100° C. for 1 h. cooled to ambient temperature, then poured onto ice and neutralized. The solid that formed was filtered and dried to give 2-(4-nitro-1H-pyrazol-3-yl)-pyridine (1.3 g) (LC/MS polar: Rₜ 2.12, [M+H]⁺192).

### 28B. Synthesis of 3-Pyridin-2-yl-1H-pyrazol-4-ylamine

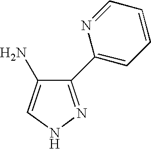

Palladium on carbon (10%, 0.08 g) was added to 2-(4-nitro-1H-pyrazol-3-yl)-pyridine (0.8 g, 4.2 mmol) partially dissolved in DMF/MeOH. The mixture was shaken under a hydrogen atmosphere at ambient temperature for 5 h, then filtered through GF/A paper and reduced in vacuo, then triturated with ether to give 3-pyridin-2-yl-1H-pyrazol-4-ylamine (0.24 g), (LC/MS polar: Rₜ 0.33,[M+H]⁺161).

### 28C. Synthesis of 5-Methanesulfonyl-2-methoxy-N-(3-pyridin-2-yl-1H-pyrazol-4-yl)benzamide

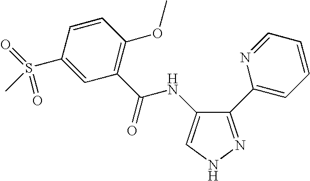

A mixture of 2-methoxy-5-methylsulfonyl benzoic acid (50 mg, 0.31 mmol), 3-pyridin-2-yl-1H-pyrazol-4-ylamine (72 mg, 0.31 mmol), EDC (72 mg, 0.37 mmol) and HOAt (51 mg, 0.37 mmol) in DMF (2 ml) was stirred at ambient temperature for 2 h. The mixture was reduced in vacuo and the residue taken up in EtOAc, washed with sat. NaHCO₃ then brine and the organic portion dried (MgSO₄) and reduced in vacuo. The residue was purified by flash column chromatography [SiO₂, EtOAc-petrol (1:2, 1:1, 2:1) then EtOAc, 0.1% Et₃N being added at the 1:2 stage] to give 5-methanesulfonyl-2-methoxy-N-(3-pyridin-2-yl-1H-pyrazol-4-yl)benzamide (49 mg),(LC/MS Acidic: Rₜ 2.45, [M+H]⁺373).

### Example 29

### Synthesis of N-(3-Pyridin-2-yl-1H-pyrazol-4-yl)-acetamide

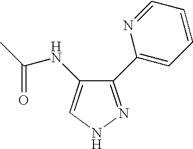

3-Pyridin-2-yl-1H-pyrazol-4-ylamine (Example 28B, 50 mg, 0.31 mmol) in THF (3 ml) with Et₃N (0.095 ml, 0.68 mmol) was cooled in an ice bath then treated with acetyl chloride (24.5 g, 0.37 mmol) and stirred 0° C. for 1 h. The solid that formed was filtered off and filtrate reduced in vacuo. The residue was purified by flash column chromatography [SiO₂, EtOAc-petrol (2:1) then EtOAc] to give N-(3-pyridin-2-yl-1H-pyrazol-4-yl)-acetamide (26 mg) (LC/MS Acidic: Rₜ 1.73, [M+H]⁺203).

### Example 30

### Synthesis of 1-(2,6-Difluoro-phenyl)-3-(3-pyridin-2-yl-1H-pyrazol-4-yl)-urea

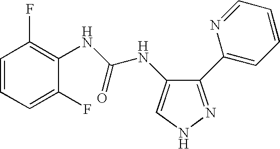

3-Pyridin-2-yl-1H-pyrazol-4-ylamine (Example 28B, 0.05 g, 0.31 mmol) was dissolved in THF (8 ml) and cooled to 0° C. and then treated with 2,6 difluorophenylisocyanate (97 mg, 0.62 mmol). The reaction was allowed to reach ambient temperature, 1M KOH (1 ml) was added and reaction stirred for 1 hour, then reduced in vacuo and the residue was partitioned between EtOAc and water, the organic layer was washed with brine, dried (MgSO₄) and reduced in vacuo and then purified by flash column chromatography [SiO₂, EtOAc-petrol (1:1, 2:1) then EtOAc] to give 1-(2,6-difluoro-phenyl)-3-(3-pyridin-2-yl-1H-pyrazol-4-yl)-urea (38 mg) (LC/MS Acidic: Rₜ 2.45, [M+H]⁺316).

### Example 31

### Synthesis of 3-Pyrazin-2-yl-1H-pyrazol-4-ylamine

### 31A. Synthyesis of 2-Bromo-1-pyrazin-2-yl-ethanone hydrobromide

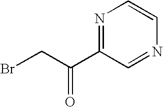

To 2-acetylpyrazine (Aldrich) (3.09 g, 25.3 mmol) was added glacial acetic acid (21.6 ml) and 30% HBr/AcOH (5 ml) followed by pyridinium tribromide (8.65 g). The slurry was stirred at ambient for 1 h then poured into ether (180 ml). The reaction was cooled and the solid formed filtered then washed with CH₃CN (×3) and Et₂O (×2) to give 2-bromo-1-pyrazin-2-yl-ethanone hydro bromide (8 g), (LC/MS Acidic: Rₜ 2.09, [M+H]⁺202).

31B. Synthesis of 2-(2-Oxo-2-pyrazin-2-yl-ethyl)-isoindole-1,3-dione

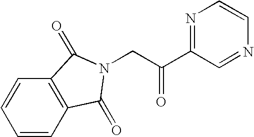

2-Bromo-1-pyrazin-2-yl-ethanone hydrobromide (1 g, 3.5 mmol) was heated at 80° C. with potassium phthalimide (1.31 g, 7.09 mmol) in DMF (10 ml) for 3 h. The reaction was partitioned between and CH₂Cl₂ and water and the aqueous re-extracted with CH₂Cl₂. The organic layers were combined and washed with 0.2M NaOH then water, dried (MgSO₄) and reduced in vacuo. Residue triturated with MeOH to give 2-(2-oxo-2-pyrazin-2-yl-ethyl)-isoindole-1,3-dione (0.125 g), (LC/MS Acidic: Rₜ 2.52, [M+H]⁺269).

### 31C. Synthesis of 3-Pyrazin-2-yl-1H-pyrazol-4-ylamine

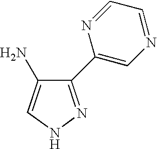

2-(2-Oxo-2-pyrazin-2-yl-ethyl)-isoindol-1,3-dione) (1.15 g, 4.29 mmol) was suspended in DMF/DMA (0.68 ml, 5.15 mmol) and heated at 100° C. for 4 h. The reaction was reduced in vacuo and used crude. The residue was dissolved in EtOH (40 ml) and treated with hydrazine hydrate (0.5 ml) and stirred at ambient O/N. Reaction was reduced in vacuo and then partitioned between EtOAc and 1M NaOH. The organic layer was washed with brine, dried (MgSO₄) and reduced in vacuo and then purified through preparative LC/MS to give 3-pyrazin-2-yl-1H-pyrazol-4-ylamine (0.3 g), (LC/MS Basic: Rₜ 0.93, [M+H]⁺162).

### Example 32

### Synthesis of 3-(5-Methyl-isoxazol-3-yl)-1H-pyrazol-4-ylamine

### 32A. Synthesis of 2-Bromo-1-(5-methyl-isoxazol-3-yl)-ethanone

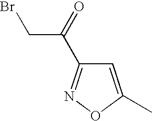

2-Bromo-1-(5-methyl-isoxazol-3-yl)-ethanone made by literature methods as described in J. Het Chem 25(1) 337-42 1988, Chiarino

### 32B. Synthesis of 3-(5-Methyl-isoxazol-3-yl)-1H-pyrazol-4-ylamine

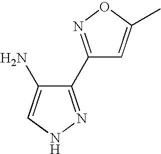

This was made in an analogous fashion to Example 31 but using 2-bromo-1-(5-methyl-isoxazol-3-yl)-ethanone (500 mg), instead of 2-bromo-1-pyrazin-2-yl-ethanone hydro bromide, to give the title compound (220 mg). (LC/MS Basic: Rₜ 1.63, [M+H]⁺165).

### Example 33

### Synthesis of 1-cyclopropyl-3-(3-pyridin-2-yl-1H-pyrazol-4-yl)-urea

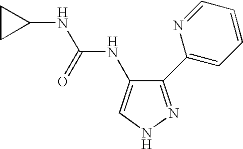

3-Pyridin-2-yl-1H-pyrazol-4-ylamine (Example 28B) (50 mg) in THF (3 ml) was treated with 1,1′-carbonyldiimidazole (200 mg) and heated at reflux for 2 hours and then allowed to cool. The resulting beige solid was collected by filtration and washed with THF. This solid was suspended in THF (1 ml), treated with cyclopropylamine (0.3 ml), and then heated at 150° C. for 15 min in a CEM Discover™ microwave synthesiser. The mixture was cooled, concentrated and then triturated with water. The product was collected by filtration and washed with diethyl ether to give 1-cyclopropyl-3-(3-pyridin-2-yl-1H-pyrazol-4-yl)-urea (20 mg) as a colourless solid. (LC/MS Basic: Rₜ 2.30, [M+H]⁺244).

### Examples 34-46

By using methods analogous to the procedure identified the compounds set out in the table below were prepared.

| | | Prepared using | | |
|---|---|---|---|---|
| | | method | | |
| | | analogous to | Differences to | |
| Example | | General | General | |
| No. | Structure | Procedure XX | Procedure? | LCMS |
| 34 | 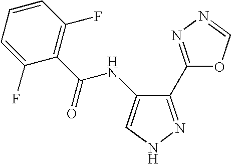 | Example 27 | Use triethylchloro- formate instead of thiocyanate | [M + H]⁺ 292 Rₜ 2.24 Acidic |
| 35 | 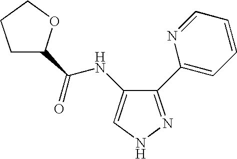 | Example 28C | | [M + H]⁺ 259 Rₜ 2.31 Acidic |
| 36 | 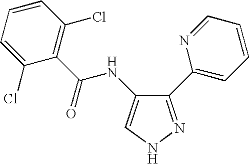 | Example 28C | Heated 80° C. for 5h Purified by Prep LC/MS | [M + H]⁺ 333 Rₜ 2.86 Acidic |
| 37 | 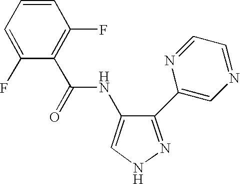 | Example 28C | But using Example 31C as starting material instead of Example 28B | [M + H]⁺ 302 Rₜ 2.49 Acidic |
| 38 | 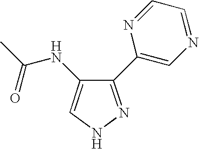 | Example 29 | But using Example 31C as starting material instead of Example 28B Purified by Prep LC/MS | [M + H]⁺ 204 Rₜ 1.81 Acidic |
| 39 | 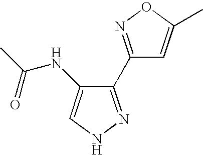 | Example 29 | But using Example 32 as starting material instead of Example 28B Purified by trituration with EtOAc | [M + H]⁺ 207 Rₜ 2.00 Acidic |
| 40 | 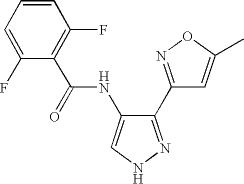 | Example 28C | But using Example 32 as starting material instead of Example 28B Purified by Prep LC/MS | [M + H]⁺ 305 Rₜ 2.72 Acidic |
| 41 | 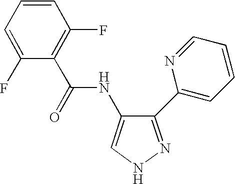 | Example 28C | RT O/N Purified by Prep LC/MS | [M + H]⁺ 301 Rₜ 2.71 Acidic |
| 42 | 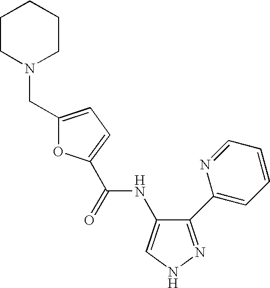 | Example 28C | Heated 80° C. for 2 hours. Purified by SiO₂ chromatography eluting with 5% MeOH— CH₂Cl₂ | [M + H]⁺ 352 Rₜ 2.00 Acidic |
| 43 | 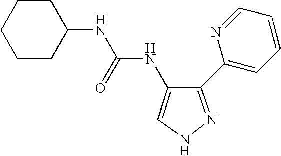 | Example 30 | Purified through preparative LC/MS | [M − H⁺]⁻ 284 Rₜ 2.59 Acidic |
| 44 | 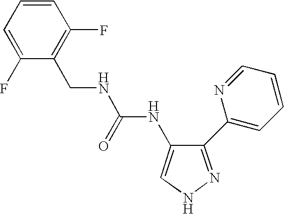 | Example 33 | 4 eq. of amine used. Reaction mixture first filtered to remove side products. Concentrated product was triturated with CH₂Cl₂ and hexanes | [M − H⁺]⁻ 328 Rₜ 2.66 Basic |

### Example 45

Synthesis of Pyrazine-2-carboxylic acid (3-pyridin-2-yl-1H-pyrazol-4-yl)-amide

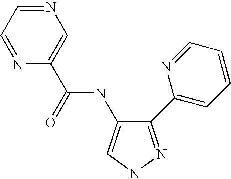

3-Pyridin-2-yl-1H-pyrazol-4-ylamine (Example 28B) (0.05 g, 0.31 mmol) was treated with 2-fluoropyrazine (0.3 ml) and heated in a CEM discover microwave synthesizer (120 W) at 140° C. until the reaction was complete. The reaction was triturated with EtOAc and the collected solid was then purified through preparative LC/MS to give pyrazine-2-carboxylic acid (3-pyridin-2-yl-1H-pyrazol-4-yl)-amide (5 mgs). (LC/MS Polar: Rₜ 2.77, [M+H]⁺239).

### Example 46

### Synthesis of 5-Chloro-2-methoxy-N-{3-[1-(2-morpholin-4-yl-ethyl)-5-oxo-4,5-dihydro-1H-1,2,4]triazol-3-yl]-1H-pyrazol-4-yl}-benzamide

### 46A. Synthesis of 4-(4-Methoxy-benzyl)-5-[1-(4-methoxy-benzyl)-4-nitro-1H-pyrazol-3-yl]-2-(2-morpholin-4-yl-ethyl)-2,4-dihydro-[1,2,4]triazol-3-one

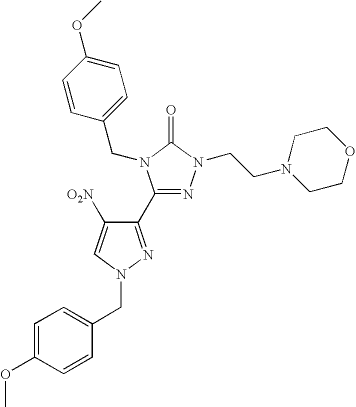

4-(4-Methoxy-benzyl)-5-[1-(4-methoxy-benzyl)-4-nitro-1H-pyrazol-3-yl]-2,4-dihydro-[1,2,4]triazol-3-one (Example 26C, 0.1 g, 0.22 mmol) was suspended in ethanol (0.5 ml) treated with 2M NaOH (0.125 ml, 0.25 mmol) followed by 4-(3-chloroethyl)-morpholine (41 mg, 0.26 mmol). The reaction mixture was heated in a CEM discover microwave synthesizer (50 W) at 70° C. until the reaction was complete, then evaporated down to give 4-(4-Methoxy-benzyl)-5-[1-(4-methoxy-benzyl)-4-nitro-1H-pyrazol-3-yl-2-(2-morpholin-4-yl-ethyl)-2,4-dihydro-[1,2,4]triazol-3-one (0.12 g) (LC/MS (acidic method): Rₜ 2.26, [M+H]⁺550).

### 46B. Synthesis of 5-[4-Amino-1-(4-methoxy-benzyl)-1H-pyrazol-3-yl]-4-(4-methoxy-benzyl)-2-(2-morpholin-4-yl-ethyl)-2,4-dihydro[1,2,4]triazol-3-one

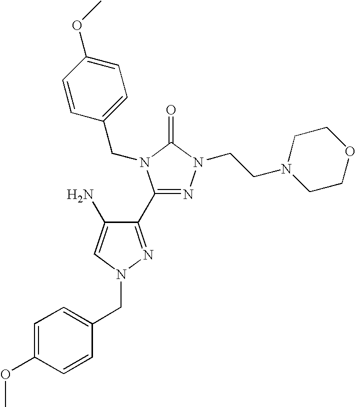

4-(4-Methoxy-benzyl)-5-[1-(4-methoxy-benzyl)-4-nitro-1H-pyrazol-3-yl]-2-(2-morpholin-4-yl-ethyl)-2,4-dihydro-[1,2,4]triazol-3-one (0.12 g, 0.21 mmol) was dissolved in methanol (20 ml) and shaken under a hydrogen atmosphere at ambient temperature for 6 h, then filtered through GF/A paper and reduced in vacuo to give 5-[4-Amino-1-(4-methoxy-benzyl)-1H-pyrazol-3-yl]-4-(4-methoxy-benzyl)-2-(2-morpholin-4-yl-ethyl)-2,4-dihydro-[1,2,4]triazol-3-one (0.122 g) (LC/MS (acidic method): Rₜ 2.23, [M+H]⁺520).

### 46C. Synthesis of 5-Chloro-2-methoxy-N-{1-(4-methoxy-benzyl)-3-[4-(4-methoxy-benzyl)-1-(2-morpholin-4-yl-ethyl)-5-oxo-4,5-dihydro-1H-[1,2,4]triazol-3-yl]-1H-pyrazol-4-yl}-benzamide

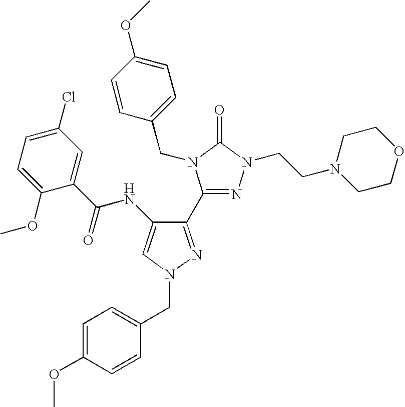

5-[4-Amino-1-(4-methoxy-benzyl)-1H-pyrazol-3-yl]-4-(4-methoxy-benzyl)-2-(2-morpholin-4-yl-ethyl)-2,4-dihydro-[1,2,4]triazol-3-one (0.123 g, 0.23 mmol), 5-chloro-2-methoxy-benzoic acid (0.048 g,0.26 mmol), EDC (0.056 g,0.26 mmol) and HOBt (0.035 g,0.26 mmol) in CH₃CN (10 ml) was stirred at ambient temperature for 16 h. The reaction was worked up by pouring into sat. NaHCO₃ and extracting with EtOAc, the organic layer was washed with water, dried (MgSO₄) and reduced in vacuo to give 5-Chloro-2-methoxy-N-{1-(4-methoxy-benzyl)-3-[4-(4-methoxy-benzyl)-1-(2-morpholin-4-yl-ethyl)-5-oxo-4,5-dihydro-1H-[1,2,4]triazol-3-yl]-1H-pyrazol-4-yl}-benzamide (0.149 g), (LC/MS Acidic: Rₜ 2.73, [M+H]⁺688).

### 46D. 5-Chloro-2-methoxy-N-{3-[1-(2-morpholin-4-yl-ethyl)-5-oxo-4,5-dihydro-1H-[1,2,4]triazol-3-yl]-1H-pyrazol-4-yl}-benzamide

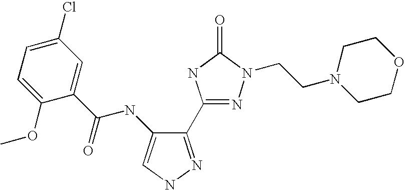

5-Chloro-2-methoxy-N-{1-(4-methoxy-benzyl)-3-[4-(4-methoxy-benzyl)-1-(2-morpholin-4-yl-ethyl)-5-oxo-4,5-dihydro-1H-[1,2,4]triazol-3-yl]-1H-pyrazol-4-yl}-benzamide (0.149 g, 0.2 mmol) was dissolved in TFA (1.5 ml) then treated with anisole (0.094 ml) and heated in a CEM discover microwave synthesizer (50 W) at 130° C. until the reaction was complete. The reaction was reduced in vacuo and triturated with MeOH, filtered and then washed with CH₂Cl₂ followed by ether. The residue was chromatographed using [SiO₂, MeOH—CH₂Cl₂ (2.5%-5%)] then treated with HCl in ether to give 5-Chloro-2-methoxy-N-{3-[1-(2-morpholin-4-yl-ethyl)-5-oxo-4,5-dihydro-1H-[1,2,4]triazol-3-yl]-1H-pyrazol-4-yl}-benzamide hydrochloride salt (0.02 g), (LC/MS Acidic: Rₜ 1.94, [M+H]⁺448).

### Example 47

### Synthesis of N-[3-(5-phenyl-2H-[1,2,4]triazol-3-yl)-1H-pyrazol-4-yl]-acetamide

### 47A. Synthesis of 1-(4-methoxy-benzyl)-4-nitro-1H-pyrazole-3-carboxylic acid ethyl ester

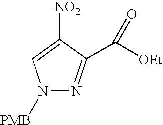

To a solution of 4-nitro-1H-pyrazole-3-carboxylic acid ethyl ester (Example 3A, 8.8 g, 47.5 mmol) in MeCN (100 ml) was added K₂CO₃ (7.9 g, 57.0 mmol) followed by 4-methoxybenzyl chloride (7.1 ml, 52.3 mmol) and the mixture stirred at ambient temperature for 20 hours. The mixture was evaporated in vacuo, the residue partitioned between EtOAc and 2M aqueous hydrochloric acid and the organic portion washed with saturated aqueous sodium hydrogen carbonate, dried (MgSO₄) and evaporated in vacuo. The residue was purified by flash column chromatography [SiO₂, EtOAc-hexane (1:4)] to give 1-(4-methoxy-benzyl)-4-nitro-1H-pyrazole-3-carboxylic acid ethyl ester (11 g) as a colourless gum.

### 47B. Synthesis of 4-Amino-1-(4-methoxy-benzyl)-1H-pyrazole-3-carboxylic acid ethyl ester

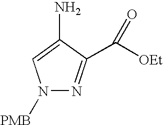

A mixture of 1-(4-methoxy-benzyl)-4-nitro-1H-pyrazole-3-carboxylic acid ethyl ester (1 g) and 10% Pd/C (100 mg) in EtOH (10 ml) was stirred under an atmosphere of hydrogen at ambient temperature and pressure for 3 hours. The catalyst was removed by filtration through Celite and the filtrate evaporated in vacuo to give 4-amino-1-(4-methoxy-benzyl)-1H-pyrazole-3-carboxylic acid ethyl ester (830 mg) as a purple gum.

### 47C. Synthesis of 4-Acetylamino-1-(4-methoxy-benzyl)-1H-pyrazole-3-carboxylic acid ethyl ester

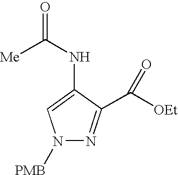

Acetic anhydride (1 ml) was added to a stirred solution of 4-amino-1-(4-methoxy-benzyl)-1H-pyrazole-3-carboxylic acid ethyl ester (1 g) in pyridine (10 ml) and the mixture stirred at ambient temperature for 16 hours. The reaction mixture was evaporated in vacuo, the residue partitioned between EtOAc and 2M hydrochloric acid and the organic portion dried (MgSO₄) and concentrated under reduced pressure to give 4-acetylamino-1-(4-methoxy-benzyl)-1H-pyrazole-3-carboxylic acid ethyl ester (1.2 g) as a pink solid.

### 47D. Synthesis of N-[3-hydrazinocarbonyl-1-(4-methoxy-benzyl)-1H-pyrazol-4-yl-acetamide

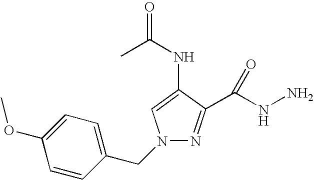

A mixture of 4-acetylamino-1-(4-methoxy-benzyl)-1H-pyrazole-3-carboxylic acid ethyl ester (320 mg; 1 mmol) and hydrazine hydrate (1 ml) in ethanol was heated in a

CEM discover microwave synthesizer at 130° C. (50 W) for 20 minutes, then evaporated and re-evaporated with toluene (×2). The residue was partitioned between EtOAc and brine. The EtOAc layer was separated, dried (MgSO₄) and evaporated to give 285 mg of N-[3-hydrazinocarbonyl-1-(4-methoxy-benzyl)-1H-pyrazol-4-yl]-acetamide as a white foam. (LC/MS Acidic: Rₜ 2.23, [M+H]⁺330).

### 47E. Synthesis of N-[1-(4-methoxy-benzyl)-3-(5-phenyl-2H-[1,2,4]triazol-3-yl)-1H-pyrazol-4-yl]-acetamide

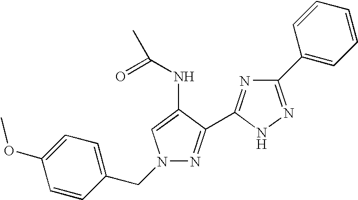

A mixture of N-[3-hydrazinocarbonyl-1-(4-methoxy-benzyl)-1H-pyrazol-4-yl]-acetamide (85 mg) and benzonitrile (1 ml) was heated at 250° C. (150 W) for 30 minutes in a CEM discover microwave synthesizer. After cooling the reaction mixture was diluted with diethyl ether and the resultant solid collected by filtration, subsequent analysis showed this to be N-[1-(4-methoxy-benzyl)-3-(5-phenyl-2H-[1,2,4]triazol-3-yl)-1H-pyrazol-4-yl]acetamide. (LC/MS Acidic: Rₜ 3.69, [M+H]⁺389).

### 47F. Synthesis of N-[3-(5-phenyl-2H-[1,2,4]triazol-3-yl)-1H-pyrazol-4-yl]-acetamide

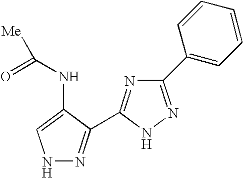

A mixture of N-[1-(4-methoxy-benzyl)-3-(4-phenyl-1H-imidazol-2-yl)-1H-pyrazol-4-yl]-acetamide (34 mg), anisole (20 ml) in TFA was heated at 120° C. (100 W) for 15 minutes in a CEM discover microwave synthesizer, then evaporated and re-evaporated with toluene (×2). The residue was purified by flash column chromatography [SiO₂, 1:1 EtOAc/hexane then EtOAc]. 8 mg of N-[3-(5-phenyl-2H-[1,2,4]triazol-3-yl)-1H-pyrazol-4-yl-acetamide were isolated as an off-white solid. (LC/MS Acidic: Rₜ 2.29, [M+H]⁺269).

### Example 48

### N-[6-(3-Pyridin-2-yl-1H-pyrazol-4-ylamino)-pyridin-3-yl]-benzamide

### 48A. N-(4-Fluoro-phenyl)-benzamide

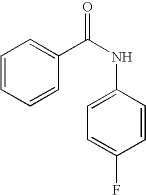

To a solution of 2-fluoropyridine (0.2 g, 1.78 mmol) in THF (20 ml) and Et₃N (0.37 ml, 2.6 mmol) was added benzoyl chloride (0.23 ml, 1.96 mmol), and the mixture was stirred at ambient temperature for 2 hours. The reaction mixture was evaporated, and the residue was partitioned between EtOAc and sat. NaHCO₃ and separated. The organic phases were washed with brine and then dried (MgSO4) and reduced in vacuo to give N-(4-Fluoro-phenyl)-benzamide, (0.3 g), (LC/MS acidic: Rt 2.49, [M+H]+217).

### 48B. N-[6-(3-Pyridin-2-yl-1H-pyrazol-4-ylamino)-pyridin-3-yl]-benzamide

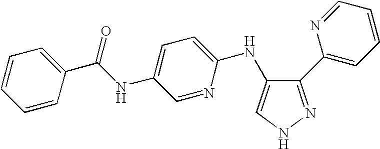

3-Pyridin-2-yl-1H-pyrazol-4-ylamine (Example 28B) (0.05 g, 0.3 mmol) was treated with N-(4-Fluoro-phenyl)-benzamide (67 mg, 0.3 mmol) and heated in a CEM Discover™ microwave synthesizer (150 W) at 150° C. until the reaction was complete. The reaction mixture was then purified through preparative LC/MS to give N-[6-(3-Pyridin-2-yl-1H-pyrazol-4-ylamino)-pyridin-3-yl]-benzamide (5 mg), (LC/MS acidic: Rt 2.1, [M+H]+357).

### Example 49

### (5-Nitro-pyridin-2-yl)-(3-pyridin-2-yl-1H-pyrazol-4-yl)-amine

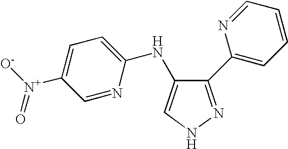

3-Pyridin-2-yl-1H-pyrazol-4-ylamine (Example 28B) (0.1 g, 0.63 mmol) was treated with 2-fluoro-5-nitro-pyridine (89 mg, 0.63 mmol) in dioxane (0.5 ml) and heated in a CEM Discover™ microwave synthesizer (150 W) at 150° C. until the reaction was complete. The reaction mixture was purified through flash column chromatography [SiO2, EtOAc-petrol (1:2) then EtOAc] to give (5-nitro-pyridin-2-yl)-(3-pyridin-2-yl-1H-pyrazol-4-yl)-amine (100 mg), (LC/MS acidic: Rt 2.85, [M+H]+357).

### Example 50

### Cyclopropanecarboxylic acid [6-(3-pyridin-2-yl-1H-pyrazol-4-ylamino)-pyridin-3-yl-amide

### 50A. N*2*-(3-Pyridin-2-yl-1H-pyrazol-4-yl)-pyridine-2,5-diamine

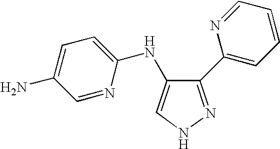

Palladium on carbon (10%, 0.01 g) was added to (5-nitro-pyridin-2-yl)-(3-pyridin-2-yl-1H-pyrazol-4-yl)-amine (0.1 g, 0.35 mmol) dissolved in MeOH (25 ml) The mixture was shaken under a hydrogen atmosphere at ambient temperature for 3 hours, then filtered through GF/A paper and reduced in vacuo, followed by trituration with ether to give N*2*-(3-Pyridin-2-yl-1H-pyrazol-4-yl)-pyridine-2,5-diamine (43 mg), (LC/MS basic: Rₜ 2.21, [M+H]⁺253).

### 50B. Cyclopropanecarboxylic acid [6-(3-pyridin-2-yl-1H-pyrazol-4-ylamino)-pyridin-3-yl]-amide

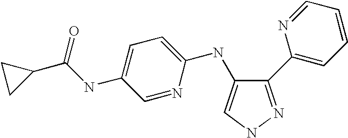

N*2*-(3-Pyridin-2-yl-1H-pyrazol-4-yl)-pyridine-2,5-diamine (21 mg, 0.08 mmol) dissolved in THF (1 ml) then treated with Et₃N (0.01 ml, 0.08 mmol) followed by cyclopropylcarbonyl chloride (8.7 mg, 0.08 mmol). The reaction mixture was stirred at ambient temperature for 1 hour, reduced in vacuo then purified through preparative LC/MS to give cyclopropanecarboxylic acid [6-(3-pyridin-2-yl-1H-pyrazol-4-ylamino)-pyridin-3-yl]-amide (12 mg), (LC/MS acidic: Rₜ 1.73, [M+H]⁺321).

### Example 51

### 1-(2,6-Difluoro-phenyl)-3-{3-[5-(4-methyl-piperazin-1-yl)-pyridin-2-yl]-1H-pyrazol-4-yl}-urea

### 51A. 5-bromo-2-(1H-pyrazol-3-yl)-pyridine

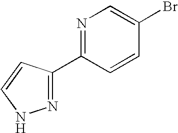

1-(5-bromo-pyridin-2-yl)-ethanone (0.9 g, 4.5 mmol) was treated with DMF-DMA (1.19 ml) and heated at 75° C. for 8 hours. The reaction mixture was reduced in vacuo and then re-evaporated with toluene. The residue was dissolved in EtOH (2 ml) and then treated with hydrazine hydrate (0.28 ml, 5.85 mmol). The reaction mixture was stirred at ambient temperature for 48 hours, reduced in vacuo, and then triturated with methanol to give the product (0.34 g). The residue was chromatographed using Biotage to give product (0.52 g) which was combined with the previous solid to give total 5-bromo-2-(1H-pyrazol-3-yl)-pyridine (0.849 g),(LC/MS acidic: Rₜ 2.36, [M+H]⁺ no ion).

### 51B. 5-Bromo-2-(4-nitro-1H-pyrazol-3-yl)-pyridine

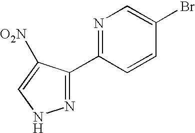

5-bromo-2-(1H-pyrazol-3-yl)-pyridine (0.849 g, 3.7 mmol) was suspended in conc. H₂SO₄ (8.5 ml) and then treated with a mixture of conc. H₂SO₄ (4.3 ml) and conc. HNO₃ (4.3 ml). The reaction mixture was stirred at ambient temperature overnight and then poured on to ice and neutralised with 2M NaOH. The solid that precipitated was filtered and washed with water to give 5-bromo-2-(4-nitro-1H-pyrazol-3-yl)-pyridine (0.8 g), (LC/MS acidic: Rₜ 2.52, [M+H]⁺269).

### 51C. 1-Methyl-4-[6-(4-nitro-1H-pyrazol-3-yl)-pyridin-3-yl]-piperazine

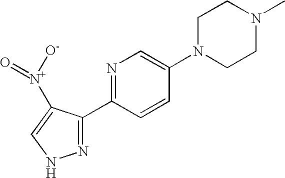

5-Bromo-2-(4-nitro-1H-pyrazol-3-yl)-pyridine (0.4 g, 1.4 mmol) was dissolved in 1-methylpiperazine and heated in a CEM discover microwave synthesizer (150 W) at 190° C. until the reaction was complete. The reaction mixture was reduced in vacuo, and then triturated with methanol to give 1-methyl-4-[6-(4-nitro-1H-pyrazol-3-yl)-pyridin-3-yl]-piperazine (0.15 g), (LC/MS basic: Rₜ 2.10, [M+H]⁺289).

### 51D. 3-[5-(4-Methyl-piperazin-1-yl)-pyridin-2-yl]-1H-pyrazol-4-ylamine

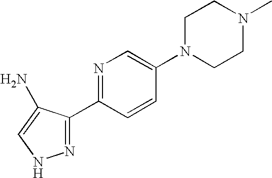

Palladium on carbon (10%, 0.05 g) was added to 1-methyl-4-[6-(4-nitro-1H-pyrazol-3-yl)-pyridin-3-yl]-piperazine (0.142 g, 0.49 mmol) dissolved in MeOH/DMF [1:1] (10 ml). The mixture was shaken under a hydrogen atmosphere at ambient temperature for 1 h, then filtered through GF/A paper and reduced in vacuo, to give 3-[5-(4-Methyl-piperazin-1-yl)-pyridin-2-yl-1H-pyrazol-4-ylamine (0.12 g), (LC/MS basic: Rₜ 1.87, [M+H]⁺259).

### 51E. 1-(2,6-Difluoro-phenyl)-3-{3-[5-(4-methyl-piperazin-1-yl)-pyridin-2-yl]-1H-pyrazol-4-yl}-urea

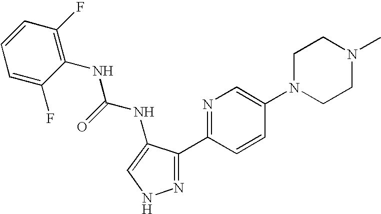

3-[5-(4-Methyl-piperazin-1-yl)-pyridin-2-yl]-1H-pyrazol-4-ylamine (0.04 g, 0.15 mmol) was suspended in THF (4 ml) and cooled to 0° C. and then treated with 2,6 difluorophenylisocyanate (48 mg, 0.30 mmol). CH₂Cl₂ and CH₃CN [1:1] (5 ml) were added and then the reaction mixture was sonicated for 5 minutes, 1M KOH (1 ml) was added and the reaction mixture was stirred for 2 hours, then reduced in vacuo.

The residue was diluted with water, and the solid was filtered off and purified by flash column chromatography [SiO₂, CMAW 120 then CMAW90]. The residue was treated with EtOAc/HCl then reduced in vacuo and triturated with ether to give 1-(2,6-Difluoro-phenyl)-3-{3-[5-(4-methyl-piperazin-1-yl)-pyridin-2-yl]-1H-pyrazol-4-yl}-urea) (25 mg), (LC/MS acidic: Rₜ 2.46, [M+H]⁺414).

### Example 52

### 3-(4-Methyl-piperazin-1-ylmethyl)-N-(3-pyridin-2-yl-1H-pyrazol-4-yl)-benzamide

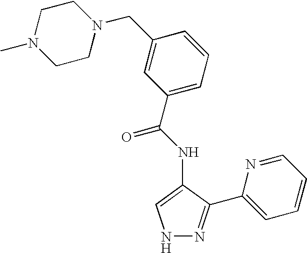

3-(4-Methyl-piperazin-1-ylmethyl)-N-(3-pyridin-2-yl-1H-pyrazol-4-yl)-benzamide was made in an analogous manner to the other pyridyl analogues herein by reacting EDC, HOAt (or HOBt), 3-pyridin-2-yl-1H-pyrazol-4-ylamine (Example 28B) and 3-(4-methyl-piperazin-1-ylmethyl)-benzoic acid in DMF. 3-(4-Methyl-piperazin-1-ylmethyl)-N-(3-pyridin-2-yl-1H-pyrazol-4-yl)-benzamide was isolated as the product following recrystallisation from AcOEt (yield=28%), (LCMS basic method, m/z=377 (MH+)+, 100%, r.t.=2.6 min).

### Biological Activity

### Example 53

Measurement of Activated CDK2/CyclinA Kinase Inhibitory Activity Assay (IC₅₀)

Compounds of the invention were tested for kinase inhibitory activity using the following protocol.

Activated CDK2/CyclinA (Brown et al, Nat. Cell Biol., 1, pp 438-443, 1999; Lowe, E. D., et al Biochemistry, 41, pp 15625-15634, 2002) is diluted to 125 pM in 2.5× strength assay buffer (50 mM MOPS pH 7.2, 62.5 mM β-glycerophosphate, 12.5 mM EDTA, 37.5 mM MgCl₂, 112.5 mM ATP, 2.5 mM DTT, 2.5 mM sodium orthovanadate, 0.25 mg/ml bovine serum albumin), and 10 μl mixed with 10 μl of histone substrate mix (60 μl bovine histone H1 (Upstate Biotechnology, 5 mg/ml), 940 μl H₂O, 35μ Ci γ³³P-ATP) and added to 96 well plates along with 5 μ of various dilutions of the test compound in DMSO (up to 2.5%). The reaction is allowed to proceed for 2 to 4 hours before being stopped with an excess of ortho-phosphoric acid (5 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

### Example 54

Measurement of Activated CDK1/Cycling Activity Assay (IC₅₀)

CDK1/CyclinB assay is identical to the CDK2/CyclinA above except that CDK1/CyclinB (Upstate Discovery) is used and the enzyme is diluted to 6.25 nM.

The compound of Example 1 has IC₅₀ values of less than 1 μM against CDK1 and 2 activity.

### Example 55

Aurora Kinase Inhibitory Activity Assay

Aurora A (Upstate Discovery) is diluted to 10 nM in 25 mM MOPS, pH 7.00, 25 mg/ml BSA, 0.0025% Brij-35, 1.25% glycerol, 0.5 mM EDTA, 25 mM MgCl₂, 0.025% β-mercaptoethanol, 37.5 mM ATP and and 10 μl mixed with 10 μl of substrate mix. The substrate mix is 500 μM Kemptide peptide (LRRASLG, Upstate Discovery) in 1 ml of water with 35 μCi γ³³P-ATP. Enzyme and substrate are added to 96 well plates along with 5 μl of various dilutions of the test compound in DMSO (up to 2.5%). The reaction is allowed to proceed for 30 minutes before being stopped with an excess of ortho-phosphoric acid (5 μl at 2%). The filtration procedure is as for Activated CDK2/CyclinA assay above.

### Example 56

Aurora A Kinase Assays

Aurora activity was determined using a Dissociative Enhanced Lanthanide Fluoro Immuno Assay (DELFIA) with a GSK3-derived biotinylated peptide. The amount of phosphorylated peptide produced is quantified by means of a phospho-specific primary antibody and europium-labelled anti-rabbit IgG antibody using time-resolved fluorescence at λ_{e×}=337 nm, λₑₘ=620 nm.

Kinase Reaction:

Assay reactions are set up in 96 well plates in a total reaction volume of 25 μl with 0.5 nM AuroraA (Upstate Discovery), 3 μM Biotin-CGPKGPGRRGRRRTSSFAEG, 15 μM ATP and various dilutions of compound in 10 mM MOPS, pH 7.0, 0.1 mg/ml BSA, 0.001% Brij-35, 0.5% glycerol, 0.2 mM EDTA, 10 mM MgCl₂, 0.01% β-mercaptoethanol & 2.5% DMSO. The reaction is allowed to proceed for 60 minutes at room temperature before stopping with 100 μl STOP buffer containing 100 mM EDTA, 0.05% Surfact-Amps20 (Pierce) and 1× Blocker™ BSA in TBS (Pierce).

Detection Step:

The reaction mixture is then transferred to a 96-well Neutravidin-coated plate (Pierce) and incubated for 30 minutes to capture the biotinylated peptide. After washing 5 times with 200 μl TBST buffer per well, a mixture of anti-phospho-(Ser/Thr)-AKT substrate antibody (Cell Signalling Technology) and Eu-N₁ anti-rabbit IgG (Perkin Elmer) is added to all wells and left for 1 hour. After a further washing step, DELFIA enhancement solution (Perkin Elmer) is added to all wells. After an incubation of 5 minutes, the wells are counted on a Fusion platereader.

### Example 57

Aurora B Kinase Assays

Aurora activity was determined using a Dissociative Enhanced Lanthanide Fluoro Immuno Assay (DELFIA) with a GSK3-derived biotinylated peptide. The amount of phosphorylated peptide produced is quantified by means of a phospho-specific primary antibody and europium-labelled anti-rabbit IgG antibody using time-resolved fluorescence at λ_{e×}337 nm, λₑₘ=620 nm.

Kinase Reaction:

Assay reactions are set up in 96 well plates in a total reaction volume of 25 μl with 5 nM AuroraB (ProQinase), 3 μM Biotin-CGPKGPGRRGRRRTSSFAEG, 15 μM ATP and various dilutions of compound in 25 mM TRIS pH 8.5, 0.1 mg/ml BSA, 0.025% Surfact-Amps 20, 5 mM MgCl₂, 1 mM DTT, & 2.5% DMSO. The reaction is allowed to proceed for 90 minutes at room temperature before stopping with 100 μl STOP buffer containing 100 mM EDTA, 0.05% Surfact-amps20 (Pierce) and 1× Blocker™ BSA in TBS (Pierce).

The detection step was carried out as described for AuroraA.

### Example 58

GSK3-B Kinase Inhibitory Activity Assay

GSK3-β (Upstate Discovery) are diluted to 7.5 nM in 25 mM MOPS, pH 7.00, 25 mg/ml BSA, 0.0025% Brij-35, 1.25% glycerol, 0.5 mM EDTA, 25 mM MgCl₂, 0.025% β-mercaptoethanol, 37.5 mM ATP and and 10 μl mixed with 10 μl of substrate mix. The substrate mix for GSK3-β is 12.5 μM phospho-glycogen synthase peptide-2 (Upstate Discovery) in 1 ml of water with 35 μCi γ³³P-ATP. Enzyme and substrate are added to 96 well plates along with 5 μl of various dilutions of the test compound in DMSO (up to 2.5%). The reaction is allowed to proceed for 3 hours (GSK3-β) before being stopped with an excess of ortho-phosphoric acid (5 μl at 2%). The filtration procedure is as for Activated CDK2/CyclinA assay above.

### Example 59

Anti-Proliferative Activity

The anti-proliferative activities of compounds of the invention were determined by measuring the ability of the compounds to inhibition of cell growth in a number of cell lines. Inhibition of cell growth was measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells were plated onto 96 well plates and allowed to recover for 16 hours prior to the addition of inhibitor compounds for a further 72 hours. At the end of the incubation period 10% (v/v) Alamar Blue was added and incubated for a further 6 hours prior to determination of fluorescent product at 535 nM ex/590 nM em. In the case of the non-proliferating cell assay cells were maintained at confluence for 96 hour prior to the addition of inhibitor compounds for a further 72 hours. The number of viable cells was determined by Alamar Blue assay as before. In addition, any morphological changes are recorded. All cell lines were obtained from ECACC (European Collection of cell Cultures).

By following the protocol set out above, compounds of the invention were found to inhibit cell growth in a number of cell lines.

Pharmaceutical Formulations

### Example 60

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

(iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

(iv) Injectable Formulation II

A parenteral compositon for injection is prepared by dissolving in water a compound of the formula (I) (e.g. in salt form) (2 mg/ml) and mannitol (50 mg/ml), sterile filtering the solution and filling into sealable 1 ml vials or ampoules.

(iv) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

(vi) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

(vii) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

(viii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

(ix) Lyophilised Formulation

Aliquots of formulated compound of formula (I) or a salt thereof as defined herein are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### Example 61

Determination of Antifungal Activity

The antifungal activity of the compounds of the formula (I) is determined using the following protocol.

The compounds are tested against a panel of fungi including Candida parpsilosis, Candida tropicalis, Candida albicans-ATCC 36082 and Cryptococcus neoformans. The test organisms are maintained on Sabourand Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 M morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The activity of the test compounds is determined using a modification of a broth microdilution technique. Test compounds are diluted in DMSO to a 1.0 mg/ml ratio then diluted to 64 μg/ml in YNB broth, pH 7.0 with MOPS (Fluconazole is used as the control) to provide a working solution of each compound. Using a 96-well plate, wells 1 and 3 through 12 are prepared with YNB broth, ten fold dilutions of the compound solution are made in wells 2 to 11 (concentration ranges are 64 to 0.125 μg/ml). Well 1 serves as a sterility control and blank for the spectrophotometric assays. Well 12 serves as a growth control. The microtitre plates are inoculated with 10 μl in each of well 2 to 11 (final inoculum size is 10⁴ organisms/ml). Inoculated plates are incubated for 48 hours at 35° C. The IC50 values are determined spectrophotometrically by measuring the absorbance at 420 nm (Automatic Microplate Reader, DuPont Instruments, Wilmington, Del.) after agitation of the plates for 2 minutes with a vortex-mixer (Vorte-Genie 2 Mixer, Scientific Industries, Inc., Bolemia, N.Y.). The IC50 endpoint is defined as the lowest drug concentration exhibiting approximately 50% (or more) reduction of the growth compared with the control well. With the turbidity assay this is defined as the lowest drug concentration at which turbidity in the well is <50% of the control (IC50). Minimal Cytolytic Concentrations (MCC) are determined by sub-culturing all wells from the 96-well plate onto a Sabourand Dextrose Agar (SDA) plate, incubating for 1 to 2 days at 35° C. and then checking viability.

### Example 62

Protocol for the Biological Evaluation of Control of in vivo Whole Plant Fungal Infection

Compounds of the formula (I) are dissolved in acetone, with subsequent serial dilutions in acetone to obtain a range of desired concentrations. Final treatment volumes are obtained by adding 9 volumes of 0.05% aqueous Tween-20™ or 0.01% Triton ^{X}100™, depending upon the pathogen.

The compositions are then used to test the activity of the compounds of the invention against tomato blight (Phytophthora infestans) using the following protocol. Tomatoes (cultivar Rutgers) are grown from seed in a soil-less peat-based potting mixture until the seedlings are 10-20 cm tall. The plants are then sprayed to run-off with the test compound at a rate of 100 ppm. After 24 hours the test plants are inoculated by spraying with an aqueous sporangia suspension of Phytophthora infestans, and kept in a dew chamber overnight. The plants are then transferred to the greenhouse until disease develops on the untreated control plants.

Similar protocols are also used to test the activity of the compounds of the invention in combatting Brown Rust of Wheat (Puccinia), Powdery Mildew of Wheat (Ervsiphe vraminis), Wheat (cultivar Monon), Leaf Blotch of Wheat (Septoria tritici), and Glume Blotch of Wheat (Leptosphaeria nodorum).

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-57. (canceled)

58. A pharmaceutical composition comprising a pharmaceutically acceptable carrier and a compound of the formula (I), or salts or tautomers or N-oxides or solvates thereof: 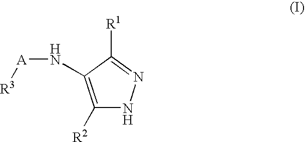
wherein
R¹ is an optionally substituted heterocyclic group having from 3 to 12 ring members provided that the cyclic group joined to the pyrazole contains at least one heteroatom selected from N, O or S;
A is a bond or —Y—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
n is 0 or 1;
Y is a bond or an alkylene chain of 1, 2 or 3 carbon atoms in length;
R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);
R³ is selected from optionally substituted carbocyclic and heterocyclic groups having from 3 to 12 ring members or an optionally substituted C₁₋₈ hydrocarbyl group;
with the proviso that R¹ is not: 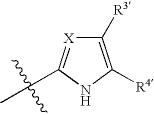
wherein
X is CR^{5′} or N;
R^{3′} and R^{4′} are the same or different and each is selected from hydrogen, CN, C(O)R^{8′}, optionally substituted C₁₋₈ hydrocarbyl and carbocyclic or heterocyclic groups having from 3 to 12 ring members; or
R^{3′} and R^{4′} together with the carbon atoms to which they are attached form an optionally substituted fused carbocyclic or heterocyclic ring having from 5 to 7 ring members of which up to 3 can be heteroatoms selected from N, O and S; and
R^{5′} is hydrogen, a group R^{2′} or a group R^{10′} wherein R^{10′} is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a′}-R^{b′} wherein R^{a′} is a bond, O, CO, X^{1′}C(X^{2′}), C(X^{2′})X^{1′}, X^{1′}C(X^{2′})X^{1′}, S, SO, SO₂, NR^{c′}, SO₂NR^{c′} or NR^{c′}SO₂; and R^{b′} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c′}, X^{1′}C(X^{2′}), C(X^{2′})X^{1′} or X^{1′}C(X^{2′})X^{1′};
R^{2′} is hydrogen, halogen, methoxy, or a C₁₋₄ hydrocarbyl group optionally substituted by halogen, hydroxyl or methoxy;
R^{c′} is selected from hydrogen and C₁₋₄ hydrocarbyl; and
X^{1′} is O, S or NR^{c′} and X² is ═O, ═S or ═NR^{c′};
R^{8′} is selected from OR^{11′}, SR^{11′} and NR^{12′}R^{13′};
R^{11′} is selected from optionally substituted C₁₋₈ hydrocarbyl and carbocyclic or heterocyclic groups having from 3 to 12 ring members; and one of R^{12′} and R^{13′} is a group R^{11′} and the other of R^{12′} and R^{13′} is hydrogen or C₁₋₄ alkyl; or R^{12′} and R^{13′} and the nitrogen atom to which they are attached together form a saturated heterocyclic group having from 4 to 7 ring members and containing 1, 2 or 3 heteroatom ring members selected from N, O and S.

59. A composition according to claim 58 wherein A is —Y—(B)ₙ—, n is 1 and B is C═O or NR^{g}(C═O) wherein R^{g} is hydrogen.

60. A composition according to claim 58 wherein Y is a bond or an alkylene chain of 1 carbon atom in length.

61. A composition according to claim 58 wherein R³ is:
(i) a monocyclic or bicyclic aromatic carbocyclic or heterocyclic group having from 5 to 10 ring members and being unsubstituted or substituted by 1 or 2 or 3 or 4 substituent groups R¹⁰; or
(ii) a non-aromatic group selected from monocyclic cycloalkyl groups and oxacycloalkyl groups wherein the non-aromatic group is unsubstituted or substituted by 1 or 2 or 3 or 4 substituent groups R¹⁰; and
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; R^{c} is selected from hydrogen and C₁₋₄ hydrocarbyl; and X¹ is O, S or NR^{c} and X² is ═O, ═S or ═NR^{c}.

62. A composition according to claim 61 wherein the substituents on R³ are selected from the group R^{30a} consisting of halogen, hydroxy, trifluoromethyl, cyano, nitro, carboxy, heterocyclic groups having 5 or 6 ring members and up to 2 heteroatoms selected from O, N and S, a group R^{a}-R^{b} wherein R^{a} is a bond, O, CO, X³C(X⁴), C(X⁴)X³, X³C(X⁴)X³, S, SO, or SO₂, and R^{b} is selected from hydrogen, heterocyclic groups having 5 or 6 ring members and up to 2 heteroatoms selected from O, N and S, and a C₁₋₄ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

63. A composition according to claim 62 wherein the substituents on R³ are selected from the group R^{30b} consisting of halogen, hydroxy, trifluoromethyl, cyano, nitro, carboxy, a group R^{a}-R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

64. A composition according to claim 58 wherein R³ is an optionally substituted C₁₋₈ hydrocarbyl group.

65. A composition according to claim 64 wherein R³ is 2,6-difluorobenzyl or unsubstituted methyl.

66. A composition according to claim 62 wherein R³ is selected from 2,6-difluorophenyl, 2,6-difluorobenzyl, 2-fluoro-6-methoxyphenyl, 2-methoxy-5-chlorophenyl, 2-methoxy-5-methanesulfonylphenyl, 2,6-dichlorophenyl, 3-fluoro-5-(4-methyl-piperazin-1-yl)-phenyl, 5-methyl-4-morpholin-4-ylmethyl-furan-2-yl, 5-piperidin-1-ylmethyl-furan-2-yl, unsubstituted methyl, unsubstituted tetrahydrofuran-2-yl; unsubstituted pyrazine such as 1, 4-pyrazin-2-yl, unsubstituted cyclopropyl and unsubstituted cyclohexyl.

67. A composition according to claim 58 wherein R¹ is an aromatic heterocycle.

68. A composition according to claim 58 wherein R¹ is selected from optionally substituted oxadiazole, optionally substituted 2,4-dihydro-[1,2,4]triazole-3-thione, optionally substituted 2,4-dihydro-[1,2,4]triazol-3-one, optionally substituted pyrazole, optionally substituted imidazo[1,2-a]pyridine, optionally substituted thiazole, optionally substituted pyridine, optionally substituted pyrazine, optionally substituted indazole, optionally substituted oxazole, optionally substituted 1H-imidazol-4-yl, and optionally substituted triazole.

69. A composition according to claim 58 wherein R¹ is substituted by 1 or 2 or 3 or 4 substituents may be selected from the group R^{10a} consisting of halogen, hydroxy, trifluoromethyl, carbocyclic and heterocyclic groups having from 3 to R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, and monocyclic non-aromatic carbocyclic or heterocyclic groups having from 3 to 6 ring members; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

70. A composition according to claim 58 wherein R¹ is substituted by 1 or 2 or 3 or 4 substituents may be selected from the group R^{10b} consisting of halogen, hydroxy, trifluoromethyl, aromatic carbocyclic and heterocyclic groups having from 3 to 10 ring members and a group R^{a}-R^{b} wherein R^{a} is a bond, O, CO, X³C(X⁴), C(X⁴)X³, X³C(X⁴)X³, and R^{b} is selected from hydrogen and a C₁₋₈ hydrocarbyl group optionally substituted by one or more substituents selected from hydroxy, oxo, halogen, and monocyclic non-aromatic carbocyclic or heterocyclic groups having from 3 to 6 ring members.

71. A composition according to claim 58 where R¹ is selected from unsubstituted [1,3,4]oxadiazol-2-yl; 4-methyl-2,4-dihydro-[1,2,4]triazole-3-thione; unsubstituted 3-(5-oxo-4,5-dihydro-1H-[1,2,4]triazol-3-yl; 2-isopropyl-2,4-dihydro-[1,2,4]triazol-3-one; 2-methyl-2,4-dihydro-[1,2,4]triazol-3-one; N-{3-[1-(2-morpholin-4-yl-ethyl)-5-oxo-4,5-dihydro-1H-[1,2,4]triazole]; N-[3-(1-isopropyl-5-oxo-4, 5-dihydro-1H-[1,2,4]triazol-3-yl, 1-methyl-5-oxo-4,5-dihydro-1H-[1,2,4]triazol-3-yl; N-{3-[1-(2-morpholin-4-yl-ethyl)-5-oxo-4,5-dihydro-1H-[1,2,4]triazol-3-yl]; unsubstituted pyrazol-4-yl; unsubstituted 1H-pyrazol-1-yl; 1-tert-butyl-1H-pyrazole, 1-methyl-1H-pyrazole; 3-phenyl-1H-pyrazole, 3-(4-fluoro-phenyl)-1H-pyrazole; 3-trifluoromethyl-1H-pyrazole; 5-trifluoromethyl-1H-pyrazole; unsubstituted 3-imidazo[1,2-a]pyridin-2-yl; 2-methyl-thiazole; 2-phenyl-thiazole; unsubstituted 2-pyridinyl; unsubstituted 1,4-pyrazin-2-yl; unsubstituted 3-indazol-2-yl; 5-methyl-isoxazole; unsubstituted 3-oxazol-5-yl; 2-methyl-oxazole; 2-trifluoromethyl-1H-imidazol-4-yl; and 3-phenyl-2H-[1,2,4]triazole.

72. A composition according to claim 58 wherein R² is hydrogen.

73. A pharmaceutical composition comprising a pharmaceutically acceptable carrier and a compound of formula (II): 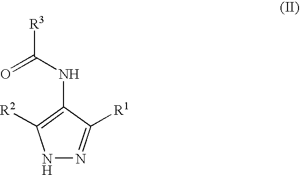
wherein R¹, R² and R³ are as defined in claim 58.

74. A composition comprising a pharmaceutically acceptable carrier and a compound of the formula (I) or a salt, N-oxide or solvate thereof as defined in claim 58, or a compound of the formula (II) or a salt, N-oxide or solvate thereof as defined in claim 73 wherein the compound is other than N-[3-(morpholin-4-yl)-5-(trifluoromethyl)-1H-pyrazol-4-yl-benzamide, 2,2,2-trifluoro-N-(3-morpholin-4-yl-5-trifluoromethyl-1H-pyrazol-4-yl)-acetamide and 4-nitro-N-[3-(piperidin-1-yl)-5-(trifluoromethyl)-1H-pyrazol-4-yl-benzamide.

75. A compound of formula (III): 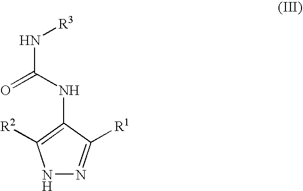
wherein R¹, R² and R³ are as defined in claim 58.

76. A compound according to claim 75 wherein R³ is cyclopropyl.

77. A method for treating a disease or condition comprising or arising from abnormal cell growth in a mammal, which method comprises administering to the mammal a compound of formula (I) as defined in claim 58 in an amount effective in inhibiting abnormal cell growth.

78. A method according to claim 77 wherein the disease state or condition is selected from proliferative disorders, viral infections, autoimmune diseases and neurodegenerative diseases.

79. A method according to claim 78 wherein the disease state or condition is a cancer.

80. A method according to claim 79 wherein the cancer is a carcinoma of the bladder, breast, colon, kidney, epidermis, liver, lung, oesophagus, gall bladder, ovary, pancreas, stomach, cervix, thyroid, prostate or skin; a hematopoietic tumour of lymphoid lineage; a hematopoietic tumour of myeloid lineage; thyroid follicular cancer; a tumour of mesenchymal origin; a tumour of the central or peripheral nervous system; melanoma; seminoma; teratocarcinoma; osteosarcoma; xeroderma pigmentoum; keratoctanthoma; thyroid follicular cancer; or Kaposi's sarcoma.

81. A method according to claim 79 wherein the cancer is a leukaemia selected from relapsed or refractory acute myelogenous leukemia, myelodysplastic syndrome, acute lymphocytic leukemia and chronic myelogenous leukemia.

82. A method according to claim 79 wherein the disease state is a cancer selected from breast cancer, ovarian cancer, colon cancer, prostate cancer, oesophageal cancer, squamous cancer, and non-small cell lung carcinomas.

83. A process for the preparation of a compound of formula (I) as defined in claim 58, which process comprises the reaction of a compound of the formula (XIII): 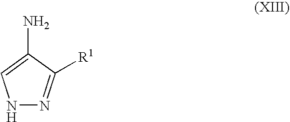
with R³—Y—CO₂H, or reactive derivative thereof and thereafter removing any protecting groups present; wherein R¹, R², and R³ are as defined in claim 58; and optionally thereafter converting one compound of the formula (I) into another compound of the formula (I).
